(19) United States
(12) Patent Application Publication
(10) Pub. No.: US 2021/0085598 A1
(43) Pub. Date: Mar. 25, 2021

(54) MICRONEEDLE COMPRISING SILK FIBROIN APPLIED TO A DISSOLVABLE BASE

(71) Applicant: VAXESS TECHNOLOGIES, INC., Vambridge, MA (US)

(72) Inventors: Kathryn M. Kosuda, Cambridge, MA (US); Jordan A. Stinson, Somerville, MA (US); Archana V. Boopathy, San Mateo, CA (US); Matthew Dirckx, Somerville, MA (US); Jonathan A. Kluge, Cambridge, MA (US); Yichen Zhang, Cambridge, MA (US); Carter R. Palmer, La Jolla, CA (US); Michael A. Schrader, Watertown, MA (US)

(21) Appl. No.: 17/044,439

(22) PCT Filed: Apr. 2, 2019
(86) PCT No.: PCT/US2019/025467

§ 371 Date: Oct. 1, 2020

Related U.S. Application Data
(60) Provisional application No. 62/652,275, filed on Apr. 3, 2018.

(51) Int. Cl. A61K 9/00; A61K 39/145

(52) U.S. Cl. CPC A61K 9/0021; A61K 2039/54; A61K 39/145

(57) ABSTRACT

Microneedle and microneedle devices including implantable silk-based tips for sustained dermal delivery of a vaccine, kits, as well as methods of manufacturing and using the same are described herein. In other embodiments, compositions and methods for controlled- or sustained-administration of a vaccine (e.g., an influenza vaccine) to provide improved immunogenicity and/or broad-spectrum immunity to a subject are also described.

### RELATED APPLICATIONS

This application claims priority to U.S. Ser. No. 62/652,275 filed Apr. 4, 2018, the contents of which is incorporated herein by reference in its entirety.

### GOVERNMENT SUPPORT

This invention was made with support from the federal government under Grant No. 1632434 awarded by the National Science Foundation as a SBIR Phase II award. The U.S. government has certain rights in the invention.

### FIELD OF THE INVENTION

The present invention generally relates to compositions and devices for achieving a controlled- or sustained-release of a vaccine in a subject, and methods of making and using the same.

### BACKGROUND OF THE INVENTION

Recent studies have shown that modulating the kinetics of antigen presentation to mimic those of a natural infection can drive more potent immune responses. The use of microneedles has been investigated in the delivery of therapeutic agents, including vaccines. Traditional materials used in the fabrication of microneedles, including silicon, metals, dextrin, glass, ceramic, maltose, galactose, and synthetic polymers, are known to be associated with various limitations that compromise their production and limit their performance (see, e.g., Donnelly et al. Drug Deliv. 17(4): 187-207, 2010).

The use of silk and silk-based materials in the fabrication of microneedles for controlled and sustained vaccine delivery has been explored to a lesser extent. However, silk fibroin has suitable properties for use in microneedle fabrication, including all-aqueous processing, mechanical strength, biocompatibility, and the ability to stabilize various macromolecules of biological origin. There remains a strong need for effective vaccine-delivery compositions, devices (e.g., silk-based microneedles), and methods capable of controlling and/or sustaining vaccine release to enhance an immune response (e.g., a cellular immune response and/or a humoral immune response) in a subject, and improved approaches to the manufacture of such compositions and devices (e.g., silk-based microneedles).

### SUMMARY OF THE INVENTION

The present invention is based, at least in part, on the discovery that modulating the kinetics of antigen presentation via, e.g., controlled- and/or sustained release compositions and devices (e.g., microneedles, e.g., silk-based microneedles, and microneedles devices) comprising a vaccine as described herein, e.g., a viral vaccine such as an influenza vaccine, can drive a more potent and/or lasting immune response (e.g., a more potent and/or lasting cellular immune response and/or humoral immune response) in a subject, e.g., as compared to the administration of single-dose or bolus administration of the vaccine. In some embodiments, controlled- or sustained-release of a vaccine as described herein can be used to achieve broad spectrum immunity in a subject.

In some embodiments, the microneedles and microneedles devices described herein demonstrate controlled- or sustained-release of a vaccine (e.g., an influenza vaccine) for at least about 1-2 weeks (e.g., for at least about 1, 2, 3, 4, 5, 6, 7, 8, 9, 10, 11, 12, 13 or 14 days), which results in one or more of improved immunogenicity, an enhanced immune response, and/or broad-spectrum immunity.

In other embodiments, methods, formulations, compositions, articles, devices, and/or preparations for administering a vaccine (e.g., an influenza vaccine) that provide improved immunogenicity, an enhanced immune response, and/or a broad-spectrum immunity to a subject are also disclosed. Accordingly, disclosed herein are compositions, preparations, devices (e.g., microneedles and microneedles devices), kits for controlled- and/or sustained release of a vaccine, in a subject, as well as methods of making and using the same.

Those skilled in the art will recognize, or be able to ascertain using no more than routine experimentation, many equivalents to the specific embodiments of the invention described herein. Such equivalents are intended to be encompassed by the following embodiments (E).

E1. A microneedle comprising:

(i) a backing,

(ii) a dissolvable base comprising a component other than poly acrylic acid (PAA), e.g., other than a solution of about 35% PAA. In embodiments, the dissolvable base comprises a component, e.g., one or more water-soluble components, having improved biocompatibility e.g., in a subject, compared to PAA. In some embodiments, the dissolvable base comprises a component, e.g., a water soluble components, that has a pH similar to that of a biological barrier into which it will be dissolved, e.g., has a pH of about 4.0-8. In embodiments, the dissolvable base comprises one, two, three, four, five, six, seven, eight, or more (e.g., all) of gelatin, polyethylene glycol (PEG), sucrose, carboxymethylcellulose (CMC), polyvinylpyrrolidone (PVP), polyvinyl alcohol (PVA), hyaluronate, maltose, and methyl cellulose. In some embodiments, the dissolvable base does not comprise a therapeutic agent, as described herein. In some embodiments, the dissolvable base is applied to the backing,

(iii) a microneedle tip, e.g., an implantable sustained-release tip, comprising a therapeutic agent and a silk fibroin. In some embodiments, the tip is applied to the dissolvable base,

wherein the microneedle is configured to implant the tip into a biological barrier, e.g., the skin of a subject, e.g., a human subject, e.g., at a depth (e.g., a max penetration depth of the distal part of tip) of between about 100 μm and about 600 μm,

wherein the tip comprises a silk fibroin, e.g., a regenerated silk fibroin and/or a recombinant silk fibroin.

In embodiments, the therapeutic agent in the tip is chosen from an antigen, an immunogen or a vaccine (e.g., an influenza vaccine). In embodiments, the therapeutic agent is present in an amount sufficient to induce an immune response, e.g., a humoral and/or cellular immune response.

E2. A microneedle comprising:

(i) a backing,

(ii) a dissolvable base comprising one, two, three, four, five, six, seven, eight, or more (e.g., all) of gelatin, polyethylene glycol (PEG), sucrose, carboxymethylcellulose (CMC), polyvinylpyrrolidone (PVP), polyvinyl alcohol (PVA), hyaluronate, maltose, and methyl cellulose applied to the backing,

(iii) a microneedle tip, e.g., an implantable sustained-release tip, comprising a silk fibroin applied to the dissolvable base,

wherein the microneedle is configured to implant the tip into the skin of a subject, e.g., a human subject, at a depth (e.g., a max penetration depth of the distal part of tip) of between about 100 μm and about 600 μm,

wherein the tip comprises a silk fibroin, e.g., a regenerated silk fibroin and/or a recombinant silk fibroin,

wherein the tip further comprises a therapeutic agent, e.g., an antigen, an immunogen or a vaccine (e.g., an influenza vaccine), e.g., in an amount sufficient to induce an immune response, e.g., a humoral and/or cellular immune response.

E3. The microneedle of embodiment E1 or E2, wherein the dissolvable base comprises one of gelatin, polyethylene glycol (PEG), sucrose, carboxymethylcellulose (CMC), polyvinylpyrrolidone (PVP), polyvinyl alcohol (PVA), hyaluronate, maltose, and methyl cellulose.
E4. The microneedle of embodiment E1 or E2, wherein the dissolvable base is comprised of two of gelatin, PEG, sucrose, CMC, PVP, PVA, hyaluronate, maltose, and methyl cellulose.
E5. The microneedle of embodiment E1 or E2, wherein the dissolvable base comprising three of gelatin, PEG, sucrose, CMC, PVP, PVA, hyaluronate, maltose, and methyl cellulose.
E6. The microneedle of embodiment E1 or E2, wherein the dissolvable base comprising four of gelatin, PEG, sucrose, CMC, PVP, PVA, hyaluronate, maltose, and methyl cellulose.
E7. The microneedle of embodiment E1 or E2, wherein the dissolvable base comprising five of gelatin, PEG, sucrose, CMC, PVP, PVA, hyaluronate, maltose, and methyl cellulose.
E8. The microneedle of embodiment E1 or E2, wherein the dissolvable base comprising six of gelatin, PEG, sucrose, CMC, PVP, PVA, hyaluronate, maltose, and methyl cellulose.
E9. The microneedle of embodiment E1 or E2, wherein the dissolvable base comprising seven of gelatin, PEG, sucrose, CMC, PVP, PVA, hyaluronate, maltose, and methyl cellulose.
E10. The microneedle of embodiment E1 or E2, wherein the dissolvable base comprising gelatin, PEG, sucrose, CMC, PVP, PVA, hyaluronate, maltose, and methyl cellulose.
E11. The microneedle of any one of the preceding embodiments, wherein the dissolvable base comprising gelatin and sucrose.
E12. The microneedle of any one of the preceding embodiments, wherein the dissolvable base comprises CMC.
E13. The microneedle of any one of the preceding embodiments, wherein the dissolvable base comprises PVP.
E14. The microneedle of any one of the preceding embodiments, wherein the dissolvable base comprises PVA.
E15. The microneedle of any one of the preceding embodiments, wherein the dissolvable base comprises about PVP and PVA.
E16. The microneedle of any one of the preceding embodiments, wherein the dissolvable base comprises PVP, PVA, and sucrose.
E17. The microneedle of any one of the preceding embodiments, wherein the dissolvable base does not comprise poly(acrylic acid) (PAA).
E18. The microneedle of any one of the preceding embodiments, wherein the implantable sustained-release tip is configured to release a vaccine into the skin of the subject over a period of time comprising at least about 4 days (e.g., about 1, 2, 3, 4, 5, 6, 7, 8, 9, 10, 11, 12, 13, or 14 or more days, e.g., between about 4 days and about 14 days, e.g., between about 1-2 weeks, about 1-3 weeks, or about 1-4 weeks).
E19. The microneedle of any one of the preceding embodiments, wherein the implantable sustained-release tip is configured to release a vaccine into the skin of the subject over a period of time comprising between about 1 week to about 2 weeks (e.g., about 1, 2, 3, 4, 5, 6, 7, 8, 9, 10, 11, 12, 13, or 14 days).
E20. The microneedle of any one of the preceding embodiments, wherein immune response comprises a cellular and/or humoral immune response comprising: (i) an elevated hemagglutination inhibition (HAI) antibody titer detectable in the blood of the subject, e.g., detectable at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization, optionally wherein the elevated HAI antibody titer is to a drifted influenza A, B, C, and/or D strain;

(ii) an elevated anti-influenza IgG titer detectable in the blood of the subject, e.g., detectable at least 1, 2, 3, 4, 5, 6, 7, 8, 9, 10, 11, and/or 12-months or more post immunization, optionally wherein the elevated anti-influenza IgG titer is to a drifted influenza A, B, C, and/or D strain; and/or

(iii) a level of antibody secreting plasma cells (ASC) against the virus, e.g., the influenza virus, e.g., the drifted influenza A, B, C, and/or D strain, detectable in the bone marrow of the subject, e.g., detectable at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization.

E21. The microneedle of any one of the preceding embodiments, wherein:

(i) an elevated hemagglutination inhibition (HAI) antibody titer is detectable in the blood of the subject for the duration of a complete flu season post immunization, optionally wherein the elevated HAI antibody titer is to a drifted influenza A, B, C, and/or D strain; and/or

(ii) the percent seroconversion, e.g., based on the elevated HAI antibody titer detectable in the blood of the subject, e.g., at 6-month post immunization is greater than about 20% (e.g., 20%, 25%, 30%, 35%, 40%, 45%, 50%, 55%, 60%, 65%, 70%, 75%, 80%, 85%, 90%, or 95% or more, e.g., 100%).

E22. The microneedle of any one of the preceding embodiments, wherein:

(i) the immune response is a cellular immune response comprising an increase in the number of IFNγ secreting cells in the blood of the subject, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization; and/or

(ii) the elevated HAI antibody titer, the elevated anti-influenza IgG titer, the level of antibody secreting plasma cells (ASC) against the virus, and/or the level of IFNγ secreting cells detectable in the subject is greater (e.g., 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold or more greater) as compared to the administration of a single-dose or a bolus administration of the vaccine.

E23. The microneedle of any one of the preceding embodiments, wherein the dissolvable base comprises between about 10% and about 70% gelatin (e.g., hydrolyzed gelatin) (e.g., about 10%, about 20%, about 30%, about 40%, about 50%, about 60%, or about 70% gelatin).
E24. The microneedle of any one of the preceding embodiments, wherein the dissolvable base comprises between about 1% and about 35% sucrose (e.g., about 1%, about 2%, about 3%, about 4%, about 5%, about 6%, about 7%, about 8%, about 9%, about 10%, about 15%, about 20%, about 25%, about 30%, or about 35% sucrose).
E25. The microneedle of any one of the preceding embodiments, wherein the dissolvable base comprises between about 1% and about 35% CMC (e.g., about 1%, about 2%, about 3%, about 4%, about 5%, about 6%, about 7%, about 8%, about 9%, about 10%, about 15%, about 20%, about 25%, about 30%, or about 35% CMC).
E26. The microneedle of any one of the preceding embodiments, wherein the dissolvable base comprises between about 10% and about 70% PVP (e.g., about 10%, about 20%, about 30%, about 40%, about 50%, about 60%, or about 70% PVP).
E27. The microneedle of any one of the preceding embodiments, wherein the dissolvable base comprises between about 1% and about 35% PVA (e.g., e.g., about 1%, about 2%, about 3%, about 4%, about 5%, about 6%, about 7%, about 8%, about 9%, about 10%, about 15%, about 20%, about 25%, about 30%, or about 35% PVA).
E28. The microneedle of any one of the preceding embodiments, wherein the dissolvable base comprises about 40% hydrolyzed gelatin and about 10% sucrose w/v.
E29. The microneedle of any one of the preceding embodiments, wherein the dissolvable base comprises up to about 50% w/v of PVP (e.g., PVP of 10 kD MW).
E30. The microneedle of any one of the preceding embodiments, wherein the dissolvable base comprises up to about 20% PVA (e.g., 87% hydrolyzed PVA at 13 kD MW).
E31. The microneedle of any one of the preceding embodiments, wherein the dissolvable base comprises CMC at up to about 10%.
E32. The microneedle of any one of the preceding embodiments, wherein the dissolvable base comprises about 1% CMC (e.g., low-viscosity CMC).
E33. The microneedle of any one of the preceding embodiments, wherein the dissolvable base comprises about 30% PVP and about 10% PVA.
E34. The microneedle of any one of the preceding embodiments, wherein the dissolvable base comprises about 37% PVP, about 5% PVA, and about 15% sucrose.
E35. The microneedle of any one of the preceding embodiments, wherein the implantable sustained-release tip comprises silk fibroin at about 1% w/v to about 10% w/v (e.g., about 1, 2, 3, 4, 5, 6, 7, 8, 9, or 10% w/v, or a silk fibroin having a molecular weight distribution according to FIG. 5, or, comprises silk fibroin in an amounta between about 20 μg to about 245 μg, e.g., per 121 microneedle array).
E36. The microneedle of any one of the preceding embodiments, wherein the implantable sustained-release tip comprises a vaccine formulated in a 1% w/v to about 10% w/v (e.g., about 1, 2, 3, 4, 5, 6, 7, 8, 9, or 10% w/v) of 10 MB silk fibroin solution, or a silk fibroin solution according to FIG. 5.
E37. The microneedle of any one of the preceding embodiments, wherein the implantable sustained-release tip comprises a vaccine formulated in a 1% w/v to about 10% w/v (e.g., about 1, 2, 3, 4, 5, 6, 7, 8, 9, or 10% w/v) of 60 MB silk fibroin solution, or a silk fibroin solution according to FIG. 5, e.g., a 100 kDa to 200 kDa (e.g., about 153 kDa) silk fibroin solution.
E38. The microneedle of any one of the preceding embodiments, wherein the implantable sustained-release tip comprises a vaccine formulated in a 1% w/v to about 10% w/v (e.g., about 1, 2, 3, 4, 5, 6, 7, 8, 9, or 10% w/v) of 120 MB silk fibroin solution, or a silk fibroin solution according to FIG. 5, e.g., a 70 kDa to 150 kDa (e.g., about 100 kDa) silk fibroin solution.
E39. The microneedle of any one of the preceding embodiments, wherein the implantable sustained-release tip comprises a vaccine formulated in a 1% w/v to about 10% w/v (e.g., about 1, 2, 3, 4, 5, 6, 7, 8, 9, or 10% w/v) of 180 MB silk fibroin solution, or a silk fibroin solution according to FIG. 5, e.g., a 36 kDa to 100 kDa (e.g., about 71 kDa) silk fibroin solution.
E40. The microneedle of any one of the preceding embodiments, wherein the implantable sustained-release tip comprises a vaccine formulated in a 1% w/v to about 10% w/v (e.g., about 1, 2, 3, 4, 5, 6, 7, 8, 9, or 10% w/v) of 480 MB silk fibroin solution, or a silk fibroin solution according to FIG. 5, e.g., a 1 kDa to 60 kDa (e.g., about 16 kDa) silk fibroin solution.
E41. The microneedle of embodiment E37, wherein the implantable sustained-release tip comprises a 5% wt/vol of 60 MB silk fibroin solution.
E42. The microneedle of any one of the preceding embodiments, wherein the implantable sustained-release tip comprises a standard human dose of a vaccine.
E43. The microneedle of any one of the preceding embodiments, wherein the standard dose of the vaccine (e.g., influenza vaccine) comprises between about 0.1 μg and about 65 μg per strain, e.g., 0.2 μg and about 50 μg per strain (e.g., about each of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 μg per strain.
E44. The microneedle of embodiment E42 or E43, wherein the implantable sustained-release tip comprises at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% or more of the standard dose.
E45. The microneedle of any one of embodiment E42-E44, wherein the implantable sustained-release tip comprises about 0.1 μg to about 65 μg of vaccine (e.g., about 0.1 μg, about 0.2 μg, about 0.3 μg, about 0.4 μg, about 0.5 μg, about 0.6 μg, about 0.7 μg, about 0.8 μg, about 0.9 μg, about 1 μg, about 1 μg to about 10 μg, about 10 μg to about 20 μg, about 20 μg to about 30 μg, about 30 μg to about 40 μg, about 40 μg to about 50 μg, about 50 μg to about 65 μg of a vaccine).
E46. The microneedle of any one of the preceding embodiments, wherein the length of the microneedle is between about 350 μm to about 1500 μm ((e.g., about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm).
E47. The microneedle of any one of the preceding embodiments, wherein the height of the implantable sustained-release tip may extend to approximately half of the full height of the microneedle.
E48. The microneedle of any one of the preceding embodiments, wherein the height of the implantable sustained-release tip is between about 75 μm to about 475 μm (e.g., about 75, about 100 μm, about 125 μm, about 150 μm, about 175 μm, about 200 μm, about 225 μm, about 250 μm, about 275 μm, about 300 μm, about 325 μm, about 375 μm, about 400 μm, about 425 μm, or about 475 μm).
E49. The microneedle of any one of the preceding embodiments, wherein the implantable sustained-release tip comprises a tip radius between about 0.5 μm to about 25 μm (e.g., about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, or 25 μm).
E50. The microneedle of any one of the preceding embodiments, wherein the implantable sustained-release tip comprises a tip radius between about 5 μm to about 10 μm (e.g., about 5, 6, 7, 8, 9, or 10 μm).
E51. The microneedle of any one of the preceding embodiments, wherein the implantable sustained-release tip comprises an angle between about 5 degrees and about 45 degrees (e.g., about 5, 6, 7, 8, 9, 10, 15, 20, 25, 30, 35, 40, or 45 degrees).
E52. The microneedle of any one of the preceding embodiments, wherein the backing is chosen from a solid support, e.g., a paper-based material, a plastic material, a polymeric material, or a polyester-based material (e.g., a Whatman 903 paper, a polymeric tape, a plastic tape, an adhesive-backed polyester tape, or other medical tape).
E53. The microneedle of any one of the preceding embodiments, wherein the implantable sustained-release tip comprises an influenza vaccine, e.g., a univalent (e.g., monovalent) or multivalent influenza vaccine (e.g., a tetravalent or quadrivalent influenza vaccine).
E54. The microneedle of embodiment E53, wherein the influenza vaccine comprises an influenza A vaccine, an influenza B vaccine, an influenza C vaccine, and/or an influenza D vaccine.
E55. The microneedle of embodiment E53 or E54, wherein the influenza vaccine comprises an influenza A vaccine, optionally wherein the influenza A vaccine is a H1N1 (e.g., A/Michigan and/or A/California) vaccine and/or a H3N2 (e.g., A/Hong Kong and/or A/Switzerland) vaccine.
E56. The microneedle of any one of embodiments E53-E55, wherein the influenza vaccine comprises an influenza B vaccine, optionally wherein the influenza B vaccine is an B/Yamagata lineage (e.g., B/Phuket) and/or the B/Victoria lineage (e.g., B/Brisbane) vaccine.
E57. The microneedle of any one of embodiments E53-E56, wherein the influenza vaccine comprises an influenza A vaccine (e.g., a H1N1 vaccine and/or a H3N2 vaccine) and an influenza B vaccine (e.g., an B/Yamagata lineage and/or the B/Victoria lineage vaccine).
E58. A device, e.g., an array or patch, comprising a plurality of microneedles (e.g., two or more microneedles as described herein), e.g., a plurality of microneedles according to any one of embodiments E1-E57.
E59. The device of embodiment E58, wherein the microneedles of the plurality are the same, e.g., comprise the same implantable sustained-release tip, e.g., comprising the same therapeutic agent, e.g., the same immunogen, antigen or vaccine.
E60. The device of embodiment E58, wherein two or more of the microneedles of the plurality are different, e.g., comprise two or more different implantable sustained-release tips, e.g., comprising two or more therapeutic agents, e.g., comprising a combination of two or more immunogens, antigens or vaccines, with or without one or more adjuvants.
E61. The device of embodiment E60, which comprises at least 10%, 20%, 30%, 40%, 50%, 60%, 70%, 80%, 90% or more of a first implantable sustained-release tip relative to a further (e.g., second, third, fourth, fifth) implantable sustained-release tip.
E62. The device of embodiment E60, wherein a total dosage amount (e.g., a standard dose) of a vaccine, antigen, and/or immunogen is divided between the plurality of microneedles (e.g., within a patch), such that the implantable controlled- or sustained-release microneedle tip can comprise less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% or more of the total dosage amount.
E63. The device of embodiment E59-E61, wherein the implantable microneedle tip comprises about 0.1 μg to about 65 μg of vaccine (e.g., about 0.1 μg, about 0.2 μg, about 0.3 μg, about 0.4 μg, about 0.5 μg, about 0.6 μg, about 0.7 μg, about 0.8 μg, about 0.9 μg, about 1 μg, about 1 μg to about 10 μg, about 10 μg to about 20 μg, about 20 μg to about 30 μg, about 30 μg to about 40 μg, about 40 μg to about 50 μg, about 50 μg to about 65 μg of a vaccine, antigen, and/or immunogen described herein).
E64. A method of providing immunity to a virus, e.g., broad spectrum immunity, in a subject comprising contacting the skin of the subject with the microneedle of any one of embodiments E1-E53, or the device of any one of embodiments E58-E63.
E65. A method of providing a controlled- or sustained-release of a vaccine, e.g., an influenza vaccine, in a subject comprising contacting the skin of the subject with the microneedle of any one of embodiments E1-E53, or the device of any one of embodiments E58-E63.
E66. A method of enhancing an immune response to a virus, e.g., an influenza virus, in a subject comprising contacting the skin of the subject with a microneedle of any one of embodiments E1-E53, or the device of any one of embodiments E58-E63.
E67. The method of any one of embodiments E64-E66, wherein the implantable sustained-release tip is configured to release a vaccine into the skin of the subject over a period of time comprising at least about 4 days (e.g., about 1, 2, 3, 4, 5, 6, 7, 8, 9, 10, 11, 12, 13, or 14 or more days, e.g., between about 4 days and about 14 days, e.g., between about 1-2 weeks, about 1-3 weeks, or about 1-4 weeks).
E68. The method of embodiment E67, wherein the implantable sustained-release tip is configured to release a vaccine into the skin of the subject over a period of time comprising between about 1 week to about 2 weeks (e.g., about 1, 2, 3, 4, 5, 6, 7, 8, 9, 10, 11, 12, 13, or 14 days).
E69. The method of any one of embodiments E64-E68, wherein immune response is a cellular and/or humoral immune response comprising:

(i) an elevated hemagglutination inhibition (HAI) antibody titer detectable in the blood of the subject, e.g., detectable at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization, optionally wherein the elevated HAI antibody titer is to a drifted influenza A, B, C, and/or D strain;

(ii) an elevated anti-influenza IgG titer detectable in the blood of the subject, e.g., detectable at least 1, 2, 3, 4, 5, 6, 7, 8, 9, 10, 11, and/or 12-months or more post immunization, optionally wherein the elevated anti-influenza IgG titer is to a drifted influenza A, B, C, and/or D strain; and/or

(iii) a level of antibody secreting plasma cells (ASC) against the virus, e.g., the influenza virus, e.g., the drifted influenza A, B, C, and/or D strain, detectable in the bone marrow of the subject, e.g., detectable at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization.

E70. The method of embodiment E69, wherein:

(i) an elevated hemagglutination inhibition (HAI) antibody titer is detectable in the blood of the subject for the duration of a complete flu season post immunization, optionally wherein the elevated HAI antibody titer is to a drifted influenza A, B, C, and/or D strain; and/or

(ii) the percent seroconversion, e.g., based on the elevated HAI antibody titer detectable in the blood of the subject, e.g., at 6-month post immunization is greater than about 20% (e.g., 20%, 25%, 30%, 35%, 40%, 45%, 50%, 55%, 60%, 65%, 70%, 75%, 80%, 85%, 90%, or 95% or more, e.g., 100%).

E71. The method of embodiment E69 or E70, wherein:

(i) the immune response is a cellular immune response comprising an increase in the level of IFNγ secreting cell in the blood of the subject, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization; And/or

(ii) the elevated HAI antibody titer, the elevated anti-influenza IgG titer, the level of antibody secreting plasma cells (ASC) against the virus, and/or the level of IFNγ secreting cells detectable in the subject is greater (e.g., 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold or more greater) as compared to the administration of a single-dose or a bolus administration of the vaccine.

E72. A method of producing a microneedle device, the method comprising:

providing a mold including a mold body with an array of needle cavities having a predefined shape, e.g., pyramid-shaped and/or conical-shaped needle cavities, formed therein;

filling tips of the needle cavities with a composition consisting of a silk fibroin, antigen solution;

drying the filled tips of the needle cavities to create releasable tips, and optionally annealing the needle tips;

filling the needle cavities of the mold with a dissolvable base solution;

drying the dissolvable base solution to create base layers for the releasable tips; and

applying a backing layer to the base layers to create a microneedle device.

E73. The method of embodiment E72, further comprising removing the microneedle device from the mold.
E74. The method of embodiment E73, wherein the microneedle device is removed by bending the mold away from the microneedle device.
E75. The method of embodiment E73, further comprising packaging microneedle devices in a container with low moisture vapor transmission rate with a desiccant to maintain between about 0% and about 50% (e.g., between about 0% and 10%, between about 10% and about 20%, between about 20% and about 30%, between about 30% and about 40%, or between about 40% and 50%, e.g., about 25%) relative humidity inside the package.
E76. The method of embodiment E72, wherein the silk fibroin, antigen solution is dispensed into each needle cavity in the mold via nanoliter printing.
E77. The method of embodiment E76, wherein filling the tips of the needle cavities includes dispensing a solution, e.g., an antigen-silk formulation into each needle cavity.
E78. The method of embodiment E72, wherein drying the filled tips of the needle cavities includes a primary drying step and a secondary drying step.
E79. The method of embodiment E72, wherein filling the needle cavities of the mold with a dissolvable base solution includes a solution of 40% w/v Hydrolyzed Gelatin and 10% w/v Sucrose in deionized water (DIW).
E80. The method of embodiment E79, wherein filling the dissolvable base solution includes subjecting the mold to a centrifuge at 3900 rpm for 2 minutes and topping off the needle cavities with 50 μL of base solution.
E81. The method of embodiment E62, further comprising an annealing step (e.g., before filling the base) after the filling the tips of the needle cavities.
E82. The method of embodiment E62, further comprising a water annealing step (e.g., before filling the base) after the filling the tips of the needle cavities
E83. The method of embodiment E62, wherein the backing layer includes one of a paper backing layer and an adhesive plastic tape.
E84. The use of a microneedle of any one of embodiments E0-E53 in a method of providing immunity to a virus, e.g., an influenza virus.
E85. The use of a microneedle of any one of embodiments E0-E53 in a method of providing a controlled- or sustained-release of a vaccine, e.g., an influenza vaccine, in a subject.
E86. The use of a microneedle of any one of embodiments E0-E53 in a method of enhancing an immune response to a virus, e.g., an influenza virus, in a subject.
E87. The microneedle of any one of embodiments E0-E53, for use as a medicament, e.g., in any of the method embodiments described herein.
E88. The microneedle of any one of the preceding embodiments, wherein the dissolvable base comprises between about 1% and about 75% (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% hyaluronate).
E89. The microneedle of any one of the preceding embodiments, wherein the dissolvable base comprises between about 1% and about 75% (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% maltose).
E90. The microneedle of any one of the preceding embodiments, wherein the dissolvable comprises between about 1% and about 75% (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% methyl cellulose).
E91. The microneedle of embodiment E1 or E2, wherein the dissolvable base comprising eight of gelatin, PEG, sucrose, CMC, PVP, PVA, hyaluronate, maltose, and methyl cellulose.
E92. The microneedle of any one of the preceding embodiments, wherein the dissolvable base comprises PEG.
E93. The microneedle of any one of the preceding embodiments, wherein the dissolvable base comprises between about 1% and about 70% PEG (e.g., about 1%, about 2%, about 3%, about 4%, about 5%, about 6%, about 7%, about 8%, about 9%, about 10%, about 20%, about 30%, about 40%, about 50%, about 60%, or about 70% PEG).
E94. A method for providing broad-spectrum immunity to a virus, e.g., an influenza virus, in a subject, said method comprising administering a vaccine (e.g., a influenza vaccine) in an amount (e.g., a dosage) and/or over a time period sufficient to result in broad-spectrum immunity to a virus, e.g., results in an immune response (e.g., a cellular immune response and/or a humoral immune response) to a drifted strain of the virus, in the subject.
E95. The method of embodiment E94, wherein the vaccine is administered in a composition for the controlled- or sustained-release of the vaccine (e.g., for the controlled- or sustained-release of one or more viral antigens as described herein).
E96. The method of embodiment E94, wherein the vaccine is administered by a device for the controlled- or sustained-release of the vaccine (e.g., for the controlled- or sustained-release of one or more viral antigens as described herein).
E97. The method of any of embodiments E94-96, wherein the vaccine is administered into a subject, e.g., in to a tissue or cavity of the subject chosen from skin, mucosa, organ tissue, muscle tissue or buccal cavity.
E98. The method of any of embodiments E94-E97, wherein the vaccine is administered in an amount (e.g., a dosage) and/or over a time period sufficient to result in one or more of:

(i) exposure in the subject to one or more antigens in the vaccine in an amount and/or period of time to result in broad spectrum immunity, e.g., to result in an immune response (e.g., a cellular immune response and/or a humoral immune response) to a drifted strain of the virus, in the subject; or

(ii) a level of one or more antigens in the subject that is substantially steady, e.g., about 20%, 15%, 10%, 5%, or 1% to an amount, e.g., minimum amount, needed to result in an immune response (e.g., a cellular immune response and/or a humoral immune response) to the one or more antigens.

E99. The method of any one of embodiments E95-98, wherein the composition or device for the controlled- or sustained-release of the vaccine is chosen from: a microneedle (e.g., a microneedle device, e.g., a microneedle patch), an implantable device (e.g., a pump, e.g., a subcutaneous pump), an injectable formulation, a depot, a gel (e.g., a hydrogel), an implant, or a particle (e.g., a microparticle and/or a nanoparticle).
E100. The method of embodiment E99, wherein the device for the controlled- or sustained-release of the vaccine comprises a microneedle or microneedle device, e.g., described herein.
E101. The method of embodiment E99, wherein the device for the controlled- or sustained-release of the vaccine comprises a pump (e.g., a subcutaneous pump).
E102. The method of embodiment E99, wherein the composition for the controlled- or sustained-release of the vaccine comprises an injectable formulation (e.g., an injectable depot formulation).
E103. The method of embodiment E100, wherein the composition for the controlled- or sustained-release of the vaccine comprises an implant.
E104. The method of embodiment E100, wherein the composition for the controlled- or sustained-release of the vaccine comprises a gel (e.g., a hydrogel).
E105. The method of any one of embodiments E100-E104, wherein the composition or device for the controlled- or sustained-release of the vaccine comprises a particle (e.g., a microparticle and/or a nanoparticle).
E106. The method of any one of embodiments E94-104, wherein the vaccine is administered, e.g., released by the composition or device for the controlled- or sustained-release of the vaccine, e.g., into the subject, in order to maintain a vaccine dosage (e.g., an antigen concentration) for a period of time sufficient to result in broad spectrum immunity, e.g., to result in an immune response (e.g., a cellular immune response and/or a humoral immune response) to a drifted strain of the virus, in the subject (e.g., wherein the period of time is about 1 to 21 days, e.g., about 5 to 10 days or about 5 to 7 days, e.g., about 1, 2, 3, 4, 5, 6, 7, 8, 9, 10, 11, 12, 13, 14, 15, 16, 17, 18, 19, 20, or 21 days).
E107. The method of embodiment E106, wherein the composition or device for the controlled- or sustained-release of the vaccine maintains antigen release and/or level in the subject over a sustained period of time.
E108. The method of embodiment E106, wherein the composition or device for the controlled- or sustained-release of the vaccine maintains a continuous or non-continuous antigen release into the subject over a sustained period of time.
E109. The method of any one of embodiments E94-E108, wherein the vaccine is administered, e.g., released by the composition or device for the controlled- or sustained-release, over a period of time comprising at least about one week, e.g., about 1-2 weeks, about 1-3 weeks, or about 1-4 weeks.
E110. The method of any one of embodiments E94-E109, wherein the vaccine is administered, e.g., released by the composition or device for the controlled- or sustained-release, over a period of time comprising at least about 4 days (e.g., about 4, 5, 6, 7, 8, 9, 10, 11, 12, 13, 14, 15, 16, 17, 18, 19, or 20 days, or more, e.g., between about 4 days and about 2 weeks, between about 4 days and about 1 week).
E111. The method of any one of embodiments E94-E111, wherein the vaccine is administered in a dosage comprising between about 0.1 μg and about 65 μg per strain, e.g., 0.2 μg and about 50 μg per strain (e.g., about each of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 μg per strain).
E112. The method of any one of embodiments E94-E111, wherein at least about 1% of the dosage of the vaccine (e.g., at least about 0.5% to about 10%, at least about 5% to about 15% at least about 10% to about 20% of the dosage), e.g., released by the composition or device for the controlled- or sustained-release of the vaccine, e.g., into the subject, is maintained over a period of time comprising at least about 4 days (e.g., about 4, 5, 6, 7, 8, 9, 10, 11, 12, 13, or 14 days, or more, e.g., between about 4 days and about 2 weeks, between about 4 days and about 1 week).
E113. The method of any one of embodiments E94-E112, wherein the vaccine is administered, e.g., released by the composition or device for the controlled- or sustained-release, in a plurality of fractional doses of a total dose (e.g., a standard dose) over a time period, e.g., such that an immune response and/or broad-spectrum immunity is achieved, wherein the amount of the vaccine administered in each of the fractional doses is no more than 1/X, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, or 100 or more, of the total dose (e.g., a standard dose) of the vaccine.
E114. The method of any one of embodiments E94-E112, wherein the vaccine is administered, e.g., released by the composition or device for the controlled- or sustained-release of the vaccine, e.g., into the skin of the subject, in a plurality of doses equivalent to a percentage of a total dose (e.g., a percentage of a standard dose) over a time period, e.g., such that broad-spectrum immunity is achieved,

wherein the amount of the vaccine administered in each of the plurality of doses is about X %, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, 100, 125, 150, 175, 200, 300, 400, or 500 or more, of the total dose (e.g., a standard dose) of the vaccine.

E115. The method of embodiment E113 or E114, wherein the vaccine is administered such that broad-spectrum immunity is achieved, e.g., such that an immune response, e.g., a cellular immune and/or humoral immune response to a drifted strain is achieved.
E116. The method of embodiment E113 or E114, wherein the vaccine is administered as two, three, four, five, six, seven, eight, nine, ten or more fractional doses.
E117. The method of any one of embodiments E113-116, wherein the total dose (e.g., the standard dose) of the vaccine is administered to achieve broad-spectrum immunity.
E118. The method of any one of embodiments E113-116, wherein less than the total dose (e.g., the standard dose) of the vaccine is administered to achieve broad-spectrum immunity.
E119. The method of any one of embodiments E113-116, wherein more than the total dose (e.g., the standard dose) of the vaccine is administered to achieve broad-spectrum immunity.
E120. The method of any one of embodiments E113-116, wherein the amount of the vaccine administered in each of the fractional doses is the same.
E121. The method of any one of embodiments E113-116, wherein the amount of the vaccine administered in each of the fractional doses is different.
E122. The method of any one of embodiments E113-121, wherein the plurality of fractional doses is administered by intramuscular injection or intradermal injection, e.g., to achieve controlled- or sustained-release of a vaccine.
E122. The method of any one of embodiments E113-E122, wherein each dose of the plurality of fractional doses is administered at least once or twice a day, at least once every two days, at least once every three days, at least once every four days, at least once every five days, at least once every 6 days, at least one a week, or at least once a month for the duration of the time period.
E124. The method of any one of embodiments E94-E123, wherein:

(i) the vaccine comprises a first influenza strain and administration of a dose of the first influenza strain to the subject results in broad-spectrum immunity to a second influenza strain (e.g., a drifted influenza strain) not present in the implantable sustained-release tip or the vaccine;

(ii) the vaccine comprises a first influenza A strain and administration of a dose of the first influenza A strain to the subject results in broad-spectrum immunity to a drifted influenza strain (e.g., a drifted influenza A, B, C, and/or D strain) not present in the implantable sustained-release tip or the vaccine;

(iii) the vaccine comprises a first influenza B strain and administration of a dose of the first influenza B strain to the subject results in broad-spectrum immunity to a drifted influenza strain (e.g., a drifted influenza A, B, C, and/or D strain) not present in the implantable sustained-release tip or the vaccine;

(iv) the vaccine comprises a first influenza C strain and administration of a dose of the first influenza C strain to the subject results in broad-spectrum immunity to a drifted influenza strain (e.g., a drifted influenza A, B, C, and/or D strain) not present in the implantable sustained-release tip or the vaccine; and/or

(v) the vaccine comprises a first influenza D strain and administration of a dose of the first influenza D strain to the subject results in broad-spectrum immunity to a drifted influenza strain (e.g., a drifted influenza A, B, C, and/or D strain) not present in the implantable sustained-release tip or the vaccine.

E125. The method of embodiment E124, wherein the first influenza A vaccine comprises:

(i) an H1N1 (e.g., A/Michigan and/or A/California) vaccine; and/or

(ii) an H3N2 (e.g., A/Hong Kong and/or A/Switzerland) vaccine.

E126. The method of embodiment E124 or E125, wherein the drifted influenza A strain comprises:

(i) an H1N1 strain (e.g., A/Michigan and/or A/California); and/or

(ii) an H3N2 strain (e.g., A/Hong Kong and/or A/Switzerland).

E127. The method of any one of embodiments E124-E126, wherein:

(i) the first influenza A vaccine comprises an H1N1 vaccine to A/Michigan and the drifted influenza A strain comprises A/California; and/or

(ii) the first influenza A vaccine comprises an H3N2 vaccine to A/Hong Kong and the drifted influenza A strain is A/Switzerland.

E128. The method of embodiment E124, wherein the first influenza B vaccine comprises:

(i) a B/Yamagata lineage strain (e.g., B/Phuket); and/or

(ii) a B/Victoria lineage strain (e.g., B/Brisbane).

E129. The method of embodiment E124 or E128, wherein:

(i) the drifted influenza B strain is a B/Yamagata lineage strain (e.g., B/Phuket); and/or

(ii) the drifted influenza B strain is a B/Victoria lineage strain (e.g., B/Brisbane).

E130. The method of any one of embodiments E124, E128, or E129, wherein the first influenza B vaccine is to the B/Victoria lineage strain B/Brisbane and the drifted influenza B strain is the B/Yamagata lineage strain B/Phuket.
E131. The method of any one of embodiments E94-E130, wherein the immune response and/or broad-spectrum immunity comprises a cellular and/or humoral immune response comprising:

(i) an elevated hemagglutination inhibition (HAI) antibody titer detectable in the blood of the subject, e.g., detectable at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization, optionally wherein the elevated HAI antibody titer is to a drifted influenza A, B, C, and/or D strain;

(ii) an elevated anti-influenza IgG titer detectable in the blood of the subject, e.g., detectable at least 1, 2, 3, 4, 5, 6, 7, 8, 9, 10, 11, and/or 12-months or more post immunization, optionally wherein the elevated anti-influenza IgG titer is to a drifted influenza A, B, C, and/or D strain; and/or

(iii) a level of antibody secreting plasma cells (ASC) against the virus, e.g., the influenza virus, e.g., the drifted influenza A, B, C, and/or D strain, detectable in the bone marrow of the subject, e.g., detectable at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization.

E132. The method of embodiment E131, wherein an elevated hemagglutination inhibition (HAI) antibody titer is detectable in the blood of the subject for the duration of a complete flu season post immunization, optionally wherein the elevated HAI antibody titer is to a drifted influenza A, B, C, and/or D strain.
E133. The method of embodiment E131, wherein the percent seroconversion, e.g., based on the elevated HAI antibody titer detectable in the blood of the subject, e.g., at 6-month post immunization is greater than about 20% (e.g., 20%, 25%, 30%, 35%, 40%, 45%, 50%, 55%, 60%, 65%, 70%, 75%, 80%, 85%, 90%, or 95% or more, e.g., 100%).
E134. The method of any one of embodiments E94-E133, wherein broad-spectrum immunity comprises a cellular immune response comprising an increase in the level of IFNγ secreting cell in the blood of the subject, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization.
E135. The method of any one of embodiments E111-E133, wherein the elevated HAI antibody titer, the elevated anti-influenza IgG titer, the level of antibody secreting plasma cells (ASC) against the virus, and/or the level of IFNγ secreting cells detectable in the subject is greater (e.g., 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold or more greater) as compared to the administration of a single-dose or a bolus administration of the vaccine.
E136. A method for providing an immune response (e.g., a cellular immune response and/or a humoral immune response) and/or a broad spectrum immunity to a virus, e.g., an influenza virus, in a subject, said method comprising administering a vaccine (e.g., a influenza vaccine) in an amount (e.g., a dosage) and/or over a time period sufficient to elicit an immune response (e.g., a cellular immune response and/or a humoral immune response) to the virus, e.g., the influenza virus, in the subject,

wherein the vaccine is administered in a composition for the controlled- or sustained-release of the vaccine (e.g., for the controlled- or sustained-release of one or more viral antigens as described herein) over a period of time comprising about 1 to about 2 weeks (e.g., about 10 days).

E137. A method of the any of the preceding embodiments, wherein the subject (e.g., the human subject) is a pediatric subject.
E138. A method of the any of the preceding embodiments, wherein the subject (e.g., the human subject) is an adult subject.
E139. A method of the any of the preceding embodiments, wherein the subject (e.g., the human subject) is an elderly subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1F are a series of graphs showing that the sustained intradermal delivery of an influenza vaccine generates improved cellular responses and stronger, longer-lasting antibody responses. Balb/c mice (n=5/group) were immunized with Fluzone HD® vaccine by intramuscular injection (IM) or the same dose injected intradermally as fractional doses over 10 days (SR or ID Sus. Rel.) or were unimmunized (Naïve). Anti-flu vaccine IgG titers were measured by ELISA over 5 months post immunization (FIGS. 1A-1B), hemagglutination inhibition titers measured using Turkey RBCs at days 28 and 56 (FIGS. 1C and 1D), and vaccine specific IFNγ+ cells in peripheral blood determined by ELISPOT at week 12 post immunization with representative images above the graph (FIGS. 1E and 1F). Data are presented as mean±SEM, n=5/group, n.s not significant, *p<0.05, **p<0.01, ***p<0.001, ****p<0.0001 with two-way ANOVA and Tukey's post test in A, two way ANOVA and Sidak's post test in B.

FIGS. 2A-2J are a series of graphs showing that immunization via controlled- or sustained-release silk microneedles improves humoral and cellular responses. Balb/c mice (n=5/group) were immunized with Fluzone HD® vaccine by either intramuscular injections (IM) or microneedles (MN) that can sustain release of the vaccine in the skin, or were unimmunized (Naïve). Following immunization, the anti-flu IgG titers were measured by ELISA (FIGS. 2A-2B). As shown in FIG. 2B, a 3-5 fold increase in titers is observed for 6 months post immunization with MN compared to IM injection. HAI titers for the 3 strains, A/Hong Kong/H3N2, A/Michigan/H1N1 and B/Brisbane were measured at months 1, 2, 3, 4 and 6 post immunization (FIGS. 2C-2H). Significantly higher HAI titers were observed with MN with complete seroconversion maintained at month 6 compared to IM injection for the two A strains and a trend towards improved seroconversion for the B lineage (FIGS. 2D, 2F, and 2H). IFNγ cellular responses in peripheral blood was also significantly higher upon MN delivery of vaccine than IM delivery (FIGS. 2E-2F). At week 4 post vaccination, significantly higher vaccine specific IFNγ+ cells in peripheral blood was determined by ELISPOT for MN delivery with representative images shown above the graph (FIGS. 2I-2J). These results demonstrate the enhanced immunogenicity of vaccination possible though microneedle delivery. Data in are presented as mean±SEM, n=5/group, *p<0.01, **p<0.01, ***p<0.001, with two-way ANOVA and Tukey's post test in FIG. 2B, two way ANOVA and Sidak's post test in FIGS. 2D, 2F, and 2H, one way ANOVA with Tukey's post test in FIG. 2J. Two way ANOVA with Tukey's post test for 2C, 2E, and 2G, One-way ANOVA with Tukey's post test for 2I. *p<0.05, ***p<0.001, **** p<0.0001.

FIG. 2K illustrates an enlarged view of a portion of a fabricated microneedle device (top panel) prior to and after application to the skin. As shown, the tips of the needles are distinct from their respective bases, and comprise fluorescently labelled silk (Silk-AF568) and antigen (antigen-AF647). The bottom panel shows that antigen release can be extended to at least six days compared to equivalent injection in mice. Without being bound by theory, the limit-of-detection for IVIS imaging (whole animal) is approximately <1% of full dose (e.g., standard dose). In some embodiments, after application of a microneedle loss of signal is measured by IVIS imaging at about 6-7 days post immunization.

FIG. 3 is a schematic drawing of the microneedle fabrication process.

FIG. 4 illustrates a completed microneedle device having an array of microneedles applied to a backing or “handle” layer.

FIG. 5 illustrates various molecular weight profiles of silk fibroin solutions useful in fabricating a microneedle described herein.

FIGS. 6A-6B are a series of graphs showing that controlled- or sustained-release of influenza vaccine generates higher HAI titers against drifted H3N2 strain of influenza. Balb/c mice were immunized with FluzoneHD at 0.5 ug/strain either by intramuscular injection (IM, gray circle) or by intradermal injections of fractional doses for a total of 10 days (SR, black diamond) or by application of the MIMIX microneedle patch (MN, black squares). Naïve mice are indicated by open triangles. HAI titers for A/Switzerland/H3N2/2013 (a strain that was not included in the vaccine) were measured at month 4 and 5 (days 120 and 150) post immunization respectively. As shown in the figure, 10 day controlled- or sustained-release of vaccine (SR) results in significantly higher titers to the drifted strain compared to equivalent intramuscular injections. Haemagglutination inhibition titers above 40 are known correlates of protection against infection. MIMIX (MN) delivery also showed a trend towards increased HAI titers with 3 out of 5 mice achieving a HAI titer of 40 compared to no animals in the IM immunized group at month 4 post immunization indicating higher correlates of protection by controlled- or sustained-release. Data are presented as mean±SEM, n=5/group, *p<0.05, ****p<0.0001 with one-way ANOVA and Tukey's post test in A and B. Dotted line indicates seroconversion.

FIGS. 7A-7B are a series of graphs showing that controlled- or sustained-release of influenza vaccine generates more long-lived plasma cells in the bone marrow against both vaccine included and drifted H3N2 strains of influenza. Balb/c mice were immunized with FluzoneHD at 0.5 ug/strain either by intramuscular injection (IM, gray bar) or by intradermal injections of fractional doses for a total of 10 days (SR, black bar). At month 8 (day 240) post immunization, animals were sacrificed and the cells from the bone marrow were isolated. A B cell ELISPOT was performed (following manufacturer's instructions, Immunospot) to measure antibody secreting plasma cells (ASC) against the vaccine included strain (A/Hong Kong/H3N2) and drifted strain (A/Switzerland/H3N2). Data demonstrates that fractional dosing of the vaccine over 10 days (SR) resulted in significantly higher number of both vaccine-specific and drifted strain specific ASCs with representative images above the graphs. Data are presented as mean±SEM, n=5/group, *p<0.05, one-way ANOVA and Tukey's post test in A and B.

FIG. 8 is a graph showing that controlled- or sustained-release of influenza vaccine generates higher HAI titers against drifted H1N1 strain of influenza. Balb/c mice were immunized with FluzoneHD at 0.5 ug/strain either by intramuscular injection (IM, gray circle), by intradermal injections of fractional doses for a total of 10 days (SR, black diamond) or by application of the MIMIX patch (MN, black square). Naïve mice are indicated by open triangles. HAI titers for A/California/7/2009/H1N1 (a strain that was not included in the vaccine) were measured at month 6 (day 180) post immunization. As shown in the figure, 10 day controlled- or sustained-release of vaccine (SR) results in significantly higher titers compared to equivalent intramuscular injections to the drifted vaccine strain. Haemagglutination inhibition titers above 40 are known correlates of protection against infection. MIMIX (MN) delivery also showed a trend towards increased HAI titers with 3 out of 5 mice achieving a HAI titer of 40 compared to 1 animal responding in the IM immunized group indicating higher correlates of protection by controlled- or sustained-release. Data are presented as mean±SEM, n=5/group, n.s. not significant, *p<0.05, **p,0.01, one-way ANOVA and Tukey's post test.

FIGS. 9A-9B are a series of graphs showing that controlled- or sustained-release of influenza vaccine generates more long-lived plasma cells in the bone marrow against both vaccine included and drifted H1N1 strains of influenza. Balb/c mice were immunized with FluzoneHD at 0.5 ug/strain either by intramuscular injection (IM, gray bar) or by intradermal injections of fractional doses for a total of 10 days (SR, black bar). At month 8 (day 240) post immunization, animals were sacrificed and the cells from the bone marrow were isolated. A B cell ELISPOT was performed (following manufacturer's instructions, Immunospot) to measure antibody secreting plasma cells (ASC) against the vaccine included strain (A/Michigan/H1N1) and not included drifted strain (A/California/H1N1). As shown in the figure, fractional dosing of the vaccine over 10 days (SR) showed a trend towards increase in both vaccine-specific and drifted strain specific ASCs with representative ELISPOT images above the graphs. Data are presented as mean±SEM, n=5/group.

FIG. 10 is a graph showing that controlled- or sustained-release of influenza vaccine generates higher HAI titers against B lineage not included in the vaccine. Balb/c mice were immunized with FluzoneHD at 0.5 ug/strain either by intramuscular injection (IM, gray bar) or by application of the MIMIX patch (MIMIX, black bar). HAI titers for B/Phuket were measured at week 7 (day 49) post immunization. B/Phuket belongs to the Yamagata lineage that was not included in the vaccine. As shown in the figure, sustained vaccine release from MIMIX showed a trend towards increase in HAI titers to this B lineage.

FIGS. 11A-11B are a series of graphs showing that controlled- or sustained-release of influenza vaccine generates more long-lived plasma cells in the bone marrow against both vaccine included and non included B lineages of influenza. Balb/c mice were immunized with FluzoneHD at 0.5 ug/strain either by intramuscular injection (IM, gray bar) or by intradermal injections of fractional doses for a total of 10 days (SR, black bar). At month 8 (day 240) post immunization, animals were sacrificed and the cells from the bone marrow were isolated. A B cell ELISPOT was performed (following manufacturer's instructions, Immunospot) to measure antibody secreting plasma cells (ASC) against the vaccine included B/lineage strain (B/Brisbane) and to the B/Yamagata lineage (B/Phuket). As shown in the figure, fractional dosing of the vaccine over 10 days (SR) resulted in significantly higher number of both vaccine-specific and drifted strain specific ASCs. Data are presented as mean±SEM, n=5/group, *p<0.05, **p<0.01, one-way ANOVA and Tukey's post test in A and B.

### DETAILED DESCRIPTION OF THE INVENTION

The present invention is based, at least in part, on the discovery that modulating the kinetics of antigen presentation to mimic that of a natural infection (e.g., a viral infection) can drive a more potent immune response (e.g., a more potent cellular and/or humoral immune response) (see, e.g., Tam et al. PNAS. 113:E6639-E6648, 2016; and Schipper at al. J. Control Release. 242:141-147, 2016). Without wishing to be bound by theory, the microneedles and microneedle devices described herein can mimic the natural process of antigen presentation (e.g., viral antigen presentation) by enabling the release, e.g., controlled- or sustained-release, of a virus-derived antigen, immunogen, and/or vaccine into a subject, e.g., into the dermis skin layer of a subject. The controlled- or sustained-release enabled by the formulations, compositions, articles, devices, and preparations, microneedles, and microneedle devices described herein can induce greater immunogenicity, an enhanced immune response (e.g., a more potent cellular and/or humoral immune response), and/or broad-spectrum immunity in a subject, as compared to the administration of single-dose or bolus administration of, e.g., a vaccine, such as an influenza vaccine.

In some embodiments, the microneedles and microneedle devices described herein can comprise an implantable controlled- or sustained-release silk-based microneedle tip that encapsulates and/or stabilizes a therapeutic agent, such as a vaccine, an antigen, and/or an immunogen (e.g., an influenza vaccine); and a dissolving base layer that supports the distal microneedle tip. Upon application of a microneedle or microneedle device, as described herein, to a biological barrier of a subject, the base layer dissolves and the silk-based microneedle tips are implanted at a predetermined depth (e.g., a max penetration depth of the distal part of tip) within the biological barrier (e.g., the dermis layer of the skin, e.g., at a depth of between about 100 μm and about 800 μm). In some embodiments, the whole tip is not embedded within, e.g., the dermis layer of the skin, e.g., at a depth of between about 100 μm and about 800 μm. The implanted tip then slowly releases the therapeutic agent over a time period sufficiently long enough to enable immunity (e.g., over a time period of at least about 4 days (e.g., about 1, 2, 3, 4, 5, 6, 7, 8, 9, 10, 11, 12, 13, or 14 or more days, e.g., between about 4 days and about 14 days, e.g., between about 1-2 weeks, about 1-3 weeks, or about 1-4 weeks, e.g., about one week, about two weeks, about three weeks, about four weeks, about five weeks, or about six weeks or more weeks). Various properties of the silk fibroin matrix comprising the implantable controlled- or sustained-release microneedle tip, including, for example, crystallinity, beta-sheet content, and molecular weight, can be modulated to tune (e.g., alter and/or modify) the release kinetics (e.g., rate of release) of a therapeutic agent, such as a vaccine, an antigen, and/or an immunogen from the microneedle tip. In some embodiments, the implantable controlled- or sustained-release microneedle tip comprises a beta-sheet content of between about 10% and about 60% (e.g., about 10%, about 20%, about 30%, about 40%, about 50%, about 60%), e.g., as based on a “crystallinity index,” e.g., a “crystallinity index” known in the art.

In some embodiments, the controlled- or sustained-release formulations, compositions, articles, devices, and preparations, comprise at least one therapeutic agent, e.g., at least one vaccine, antigen, and/or immunogen described herein. In some embodiments, the formulations, compositions, articles, devices, and preparations for controlled- and/or sustained release described herein release a therapeutic agent (e.g., a vaccine) over a time period sufficiently long enough to enable immunity (e.g., over a time period of at least about 1 to about 14 days (e.g., about 1, 2, 3, 4, 5, 6, 7, 8, 9, 10, 11, 12, 13, or 14 or more days, e.g., between about 4 days and about 14 days, e.g., between about 1-2 weeks, about 1-3 weeks, or about 1-4 weeks, e.g., about one week, about two weeks, about three weeks, about four weeks, about five weeks, or about six weeks or more weeks). Accordingly controlled- or sustained-release formulations, compositions, articles, devices, and preparations, microneedles, microneedle devices, kits, as well as methods of making and using the same are disclosed.

### Definitions

All scientific and technical terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, in order to more clearly and concisely describe the subject matter which is the invention, the following definitions are provided for certain terms which are used in the specification and appended claims.

The articles “a” and “an” are used herein to refer to one or to more than one (i.e., to at least one) of the grammatical object of the article. By way of example, “an element” means one element or more than one element.

As used herein, the term “about” means+/−10% of the recited value.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

As used herein, an “adjuvant” is a substance that is able to favor or amplify the cascade of immunological events, ultimately leading to an increased immunological response, e.g., the integrated bodily response to an antigen, including cellular and/or humoral immune responses. Non-limiting examples of adjuvants include: aluminum (e.g., aluminum gels and/or aluminum salts, such as aluminum hydroxide, aluminum phosphate, and aluminum potassium sulfate), lipids (e.g., squalene, monophosphoryl lipid A (MPL)), AS03 (e.g., an adjuvant comprising D,L-alpha-tocopherol (vitamin E), squalene, and polysorbate 80), AS04 (e.g., an adjuvant comprising a combination of aluminum hydroxide and MPL), and MF59® (e.g., an adjuvant comprising squalene).

As used herein, the term “antigen” refers to refers to a molecule capable of inducing a humoral immune response and/or cellular immune response, e.g., leading to the activation of B and/or T lymphocytes and/or innate immune cells and/or antigen presenting cells. Any macromolecule, including almost all proteins or peptides, can be an antigen. Antigens can also be derived from genomic and/or recombinant DNA. For example, any DNA comprising a nucleotide sequence or a partial nucleotide sequence that encodes a protein capable of eliciting an immune response encodes an “antigen.” In some embodiments, an antigen does not need to be encoded solely by a full length nucleotide sequence of a gene, nor does an antigen need to be encoded by a gene at all. In some embodiments, an antigen can be synthesized or can be derived from a biological sample, e.g., a tissue sample, a tumor sample, a cell, or a fluid with other biological components. In some embodiments, an antigen can be derived from a virus. Antigens as used herein may also be mixtures of several individual antigens.

As used herein, the term “backing” refers to a material that is suitable for bonding to and/or adhering to a component of a microneedle. In some embodiments, a backing material is suitable for bonding to and/or adhering to the dissolvable base of a microneedle described herein.

As used herein, the phrase “broad-spectrum immunity” refers to an immune response, e.g., a humoral and/or cellular response (e.g., immunity or protective immunity), against at least one (e.g., against at least two, at least three, at least four, at least five, against at least eight, or at least against more than eight) strains of a virus (e.g., a virus described herein), wherein the at least one strain is not present in a vaccine administered to a subject, e.g., according to the methods, microneedles, and microneedle devices described herein. In some embodiments, the at least one strain not present in the vaccine is a drifted strain of the virus. In some embodiments, the at least one strain belongs to a different type as the strain(s) present in the vaccine.

As used herein, the term “immunity” or “protective immunity” refers to an immune response, e.g., a humoral and/or cellular response, elicited by a vaccine or immunization schedule (e.g., vaccination regimen) that when administered to a subject in need thereof (e.g., a subject described herein), that prevents, retards the development of, and/or reduces the severity of a viral infection that is caused by a virus described herein. In some embodiments, immunity or protective immunity diminishes or altogether eliminates the symptoms of the viral infection. In some embodiments, immunity or protective immunity is characterized by the presence of one or more of: circulating antibodies (e.g., humoral immunity), the presence of sensitized T lymphocytes (e.g., cellular immunity), the presence of secretory IgA on mucosal surfaces (e.g., mucosal immunity), or a combination thereof.

As used herein, the term “antigenic drift” refers to a mutation in the gene of an influenza virus that accumulates over time as the virus replicates. These mutations usually produce viruses that are closely related to one another (e.g., located close together on a phylogenetic tree), and referred to herein as “drifted strains.” In some embodiments, viruses that are closely related to each other share similar antigenic properties and an immune system exposed to a first virus and, subsequently, a drifted strain of the first virus will usually recognize the drifted strain and respond to it by mounting an immune response (e.g., a protective immune response), referred to as “cross-protection.” However, in some embodiments these small genetic changes can accumulate over time and result in viruses that are antigenically different (e.g., located further away on a phylogenetic tree), and when this happens, the body's immune system may not recognize those viruses (e.g., those drifted strains).

As used herein, the term “dissolvable base” refers to the layer that forms the base of the microneedles (e.g., functions as the support for the distal implantable silk tips that are loaded with a vaccine, an antigen, or an immunogen), and/or can also serve as a layer connecting adjacent microneedles to form a continuous microneedle array or microneedle patch. In some embodiments, at least 10%, 20%, 30%, 40%, 50%, 60%, 70%, 80%, 90% or more of the base is dissolved after application to a biological barrier, e.g., skin or mucous surface, or buccal cavity.

As used herein, the term “dose” means the amount of a vaccine, antigen, and/or immunogen which is administered (e.g., in a vaccination) to elicit an immune response (e.g., a humoral and/or a cellular immune response) in an organism.

As used herein, a “standard dose” means the amount of antigen in a typical human dose of a vaccine, as approved for marketing by national or international regulatory authorities (e.g., U.S. FDA, EMEA).

As used herein, a “fractional dose” refers to a dosage comprising a portioned amount of a total dose (e.g., a standard dose) of a vaccine, antigen, and/or immunogen which is administered (e.g., in a vaccination) to elicit an immune response (e.g., a humoral immune response, a cellular immune response, and/or a broad-spectrum immunity) in an organism. In some embodiments, the amount of the vaccine, antigen, and/or immunogen in the fractional dose is no more than 1/X, wherein X is any number, e.g., wherein X 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, or 100 or more, of the total dose (e.g., a standard dose) of the vaccine.

As used herein, the term “gelatin” refers to a water-soluble protein derived from collagen. In some embodiments, the term “gelatin” refers to a sterile nonpyrogenic protein preparation (e.g., fractions) produced by partial acid hydrolysis (type A gelatin) or by partial alkaline hydrolysis (type B gelatin) of animal collagen, most commonly derived from cattle, pig, and fish sources. Gelatin can be obtained in varying molecular weight ranges. Recombinant sources of gelatin may also be used.

As used herein, the term “polyethylene glycol (PEG)” refers to an oligomer or polymer of ethylene oxide. PEG is also known as polyethylene oxide (PEO) or polyoxyethylene (POE). The structure of PEG is commonly expressed as H—(O—CH₂—CH₂)ₙ—OH.

As used herein, the term “immunogen” refers to any substance (e.g., an antigen, combination of antigens, pathogen fragment, whole pathogen) capable of eliciting an immune response in an organism. An “immunogen” is capable of inducing an immunological response against itself after administration to a mammalian subject. The term “immunological” as used herein with respect to an immunological response, refers to the development of a humoral (antibody mediated) and/or a cellular (mediated by antigen-specific T cells or their secretion products) response directed against an immunogen in a recipient subject. Such a response can be an active response induced by administration of an immunogen or immunogenic peptide to a subject or a passive response induced by administration of antibody or primed T cells that are directed towards the immunogen. In some embodiments, an immunogen is an influenza virus. In some embodiments, an immunogen is a viral vaccine (e.g., a monovalent (also called univalent) or a multivalent (also called polyvalent) vaccine, such as for influenza). In some embodiments, the vaccine (e.g., influenza vaccine may be tetravalent or quadrivalent). In some embodiments, the immunogen is a replicating or non-replicating vaccine vector (e.g., comprises an adenovirus vector, an adeno-associated virus vector, an alpha virus vector, a herpesvirus vector, a measles virus vector, a poxvirus vector, or a vesicular stomatitis virus vector). In some embodiments, the immunogen is an enterovirus, a flavivirus, a rotavirus, a measles virus, a mumps virus, a rubella virus, or a fragment thereof. In some embodiments, an inactivated or live attenuated polio virus, or antigenic fragment thereof, is an immunogen. In some embodiments, an inactivated or live attenuated rotavirus, or antigenic fragment thereof, is an immunogen. In some embodiments, an inactivated, live attenuated or recombinant flavivirus, or antigenic fragment thereof, is an immunogen.

As used herein, the term “immunogenicity” refers to the ability of a substance, such as an antigen or epitope, to provoke humoral and/or cell-mediated immunological response in a subject. A skilled artisan can readily measure immunogenicity of a substance. The presence of a cell-mediated immunological response can be determined by any art-recognized methods, e.g., proliferation assays (CD4+ T cells), CTL (cytotoxic T lymphocyte) assays, or immunohistochemistry with tissue section of a subject to determine the presence of activated cells such as monocytes and macrophages after the administration of an immunogen. One of skill in the art can readily determine the presence of humoral-mediated immunological response in a subject by any well-established methods. For example, the level of antibodies produced in a biological sample such as blood can be measured by western blot, ELISA or other methods known for antibody detection.

As used interchangeably herein, the terms “implantable sustained-release tip” or “releasable tip” refers to the distal end, e.g., tip, of a microneedle capable of piercing a biological barrier, e.g., the skin, mucous surface, or buccal cavity, of a subject and being deposited within the biological barrier, a skin layer (e.g., the dermis). In embodiments, the tip comprises a silk fibroin protein in an amount sufficient to sustain the release of a therapeutic agent, such as a vaccine, antigen, and/or immunogen for a prolonged period of time, e.g., for at least about 4 days (e.g., about 1, 2, 3, 4, 5, 6, 7, 8, 9, 10, 11, 12, 13, or 14 or more days, e.g., between about 4 days and about 14 days, e.g., between about 1-2 weeks, about 1-3 weeks, or about 1-4 weeks). In some embodiments, the implantable sustained-release tip comprises an influenza vaccine, antigen, and/or immunogen.

As used herein, the term “microneedle” refers to a structure having at least two, more typically, three components, e.g., layers, for transport or delivery of a therapeutic agent, such as a vaccine, an antigen, and/or an immunogen, across a biological barrier, such as the skin, tissue, or cell membrane. In some embodiments, a microneedle comprises a base (e.g., a dissolvable base as described herein), a tip (e.g., an implantable tip as described herein), and optionally, a backing material. In embodiments, a microneedle has dimension of between about 350 μm to about 1500 μm in height (e.g., between about 350 μm to about 1500 μm, e.g., about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm)). In some embodiments, the microneedle is fabricated to have any dimension and/or geometry to enable the deployment of an implantable sustained-release at a depth between about 100 μm and about 900 μm (e.g., at a depth of about 800 μm) into the dermis layer of the skin for controlled- or sustained-release of a vaccine.

As used herein, the term “microneedle patch” and “microneedle array” refers to a device comprising a plurality of microneedles, e.g., silk fibroin-based microneedles, e.g., arranged in a random or predefined pattern, such as an array.

As used herein, the term “silk fibroin” includes silkworm fibroin and insect or spider silk protein. Any type of silk fibroin can be used according to various aspects described herein. Silk fibroin produced by silkworms, such as Bombyx mori, is the most common and represents an earth-friendly, renewable resource. For instance, silk fibroin used in a microneedle (e.g., an implantable controlled- or sustained-release tip of a microneedle) may be obtained by removing sericin from the cocoons of B. mori. In some embodiments, the silk fibroin is a regenerated silk fibroin, e.g., a silk fibroin obtained after extraction of sericin from the cocoons of B. mori, and an additional processing e.g. via a boiling step. Organic silkworm cocoons are also commercially available. There are many different silks, however, including spider silk (e.g., obtained from Nephila clavipes), transgenic silks, recombinant and/or genetically engineered silks, such as silks from bacteria, yeast, mammalian cells, transgenic animals, or transgenic plants (see, e.g., WO 97/08315; U.S. Pat. No. 5,245,012), and variants thereof, that can be used.

As used herein, a “subject” refers to a human or animal. Usually the animal is a vertebrate such as a primate, rodent, domestic animal or game animal. Primates include chimpanzees, cynomolgus monkeys, spider monkeys, and macaques (e.g., Rhesus). Rodents include mice, rats, woodchucks, ferrets, rabbits and hamsters. Domestic and game animals include cows, horses, pigs, deer, bison, buffalo, feline species (e.g., domestic cat), canine species (e.g., dog, fox, wolf), avian species (e.g., chicken, emu, ostrich), and fish (e.g., trout, catfish and salmon). In certain embodiments of the aspects described herein, the subject is a mammal (e.g., a primate, e.g., a human). A subject can be male or female. In certain embodiments, the subject is a mammal. The mammal can be a human, non-human primate, mouse, rat, dog, cat, horse, or cow, but are not limited to these examples. In addition, the methods and formulations described herein can be used to treat domesticated animals and/or pets.

As used herein, the term “controlled- or sustained-release” refers to the release of a therapeutic agent (e.g., from a microneedle, microneedle device, formulation, composition, article, device, and preparation described herein, e.g., from a silk fibroin-based microneedle tip as described herein), such as a vaccine, antigen, and/or immunogen over a period of time, e.g., for at least about 1-14 days (e.g., about 1, 2, 3, 4, 5, 6, 7, 8, 9, 10, 11, 12, 13, or 14 or more days, e.g., between about 4 days and about 14 days, e.g., between about 1-2 weeks, about 1-3 weeks, or about 1-4 weeks). In some embodiments, the controlled- or sustained-release of an vaccine, e.g., over a time period of about 1 to about 14 days, e.g., about 1, 2, 3, 4, 5, 6, 7, 8, 9, 10, 11, 12, 13, or 14 days, by a microneedle, microneedle device, formulation, composition, article, device, or preparation as described herein can result, e.g., in broad-spectrum immunity in a subject. In some embodiments, the vaccine formulations and preparations comprising silk fibroin have controlled- or sustained-release properties (e.g., are formulated and/or configured to release a vaccine, e.g., into the skin of the subject, over a period of, or at least 1, 5, 10, 15, 30, 45 minutes; a period of, or at least, 1, 2, 3, 4, 5, 10, 24 hours; a period of, or at least, 1, 2, 3, 4, 5, 6, 7, 8, 9, 10, 11, 12, 13, 14 days; a period of, or at least, 1, 2, 3, 4, 5, 6, 7, 8 weeks; a period of, or at least 1, 2, 3, 4, 5, 6, 7, 8, 9, 10, 11 months; a period of, or at least, 1, 2, 3, 4, 5 years, or longer.

As used herein, the term “vaccine” refers to any preparation of an antigen (including subunit antigens, toxoid antigens, conjugate antigens, or other types of antigenic molecules) or a killed or live attenuated microorganism that, when introduced into a subject's body, affects the immune response to the specific antigen or microorganism by causing activation of the immune system against the specific antigen or microorganism (e.g., inducing antibody formation, T cell responses, and/or B-cell responses). Generally, vaccines against microorganisms are directed toward at least part of a virus, bacteria, parasite, mycoplasma, or other infectious agent.

As used herein, the term “viruses” refers to an infectious agent composed of a nucleic acid encapsidated in a protein. Such infectious agents are incapable of autonomous replication (i.e., replication requires the use of the host cell's machinery). Viral genomes can be single-stranded (ss) or double-stranded (ds), RNA or DNA, and can or cannot use reverse transcriptase (RT). Additionally, ssRNA viruses can be either sense (+) or antisense (−). Exemplary viruses include, but are not limited to, dsDNA viruses (e.g., Adenoviruses, Herpesviruses, Poxviruses), ssDNA viruses (e.g., Parvoviruses), dsRNA viruses (e.g., Reo viruses), (+)ssRNA viruses (e.g., Picomaviruses, Toga viruses), (−)ssRNA viruses (e.g., Orthomyxoviruses, Rhabdoviruses), ssRNA-RT viruses, i.e., (+)sense RNA with DNA intermediate in life-cycle (e.g., Retroviruses), and dsDNA-RT viruses (e.g., Hepadnaviruses). In some embodiments, viruses can also include wild-type (natural) viruses, killed viruses, live attenuated viruses, modified viruses, recombinant viruses or any combinations thereof. Exemplary retroviruses include human immunodeficiency virus (HIV). Other examples of viruses include, but are not limited to, enveloped viruses, respiratory syncytial viruses, non-enveloped viruses (e.g., human papillomavirus (HPV)), bacteriophages, recombinant viruses, and viral vectors. The term “bacteriophages” as used herein refers to viruses that infect bacteria.

As used herein, the term “influenza virus” refers to a negative-sense ssRNA virus within the Orthomyxoviridae family. An influenza virus can be a live wild-type virus, a live attenuated virus, an inactivated virus, a chimeric virus, or a recombinant virus. Examples of influenza viruses include influenza A, influenza B, and influenza C.

As used herein, the term “therapeutic agent” is art-recognized and refers to any chemical moiety that is a biologically, physiologically, or pharmacologically active substance that acts locally or systemically in a subject. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness, such as a viral infection; substances which affect the structure or function of the body; or pro-drugs, which become biologically active or more active after they have been placed in a physiological environment. Various forms of a therapeutic agent may be used which are capable of being released from the microneedles described herein into adjacent tissues or fluids upon administration to a subject. Examples include steroids and esters of steroids (e.g., estrogen, progesterone, testosterone, androsterone, cholesterol, norethindrone, digoxigenin, cholic acid, deoxycholic acid, and chenodeoxycholic acid), boron-containing compounds (e.g., carborane), chemotherapeutic nucleotides, drugs (e.g., antibiotics, antivirals, antifungals), enediynes (e.g., calicheamicins, esperamicins, dynemicin, neocarzinostatin chromophore, and kedarcidin chromophore), heavy metal complexes (e.g., cisplatin), hormone antagonists (e.g., tamoxifen), non-specific (non-antibody) proteins (e.g., sugar oligomers), oligonucleotides (e.g., mRNA sequences or antisense oligonucleotides that bind to a target nucleic acid sequence), peptides, proteins, antibodies, photodynamic agents (e.g., rhodamine 123), radionuclides (e.g., I-131, Re-186, Re-188, Y-90, Bi-212, At-211, Sr-89, Ho-166, Sm-153, Cu-67 and Cu-64), toxins (e.g., ricin), and transcription-based pharmaceuticals.

### Microneedles Devices

The invention provides, silk fibroin-based microneedles and microneedle devices (e.g., microneedle arrays and patches) for the transport and release, e.g., controlled- or sustained-release, of a therapeutic agent, such as a vaccine, an antigen, and/or an immunogen (e.g., an influenza vaccine) across a biological barrier, such as the skin, a mucous membrane, a buccal cavity, a tissue, or a cell membrane. The microneedles described herein can be in any shape and/or geometry suitable for use in piercing a biological barrier, e.g., a layer of the skin, to enable release, e.g., controlled- or sustained-release, of a vaccine within a subject. Non-limiting examples of the shape and/or geometry of the microneedles include: a cylindrical shape, a wedge-shape, a cone-shape, a pyramid-shape, and/or an irregular-shape, or any combinations thereof.

In some embodiments, a microneedle of the invention can comprise the following layers: (1) a backing material; (2) a dissolvable base; and (3) an implantable controlled- or sustained-release tip. For example, the microneedles described herein may include a backing material applied to a dissolvable base layer that supports a distal controlled- or sustained-release implantable tip comprising a silk fibroin and vaccine (e.g., an influenza vaccine, antigen, and/or immunogen).

In some embodiments, the length of the microneedle can be between about 350 μm to about 1500 μm ((e.g., about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm). In embodiments, the length of microneedles can be fabricated sufficiently long enough to enable delivery of an implantable tip comprising a vaccine, an antigen, and/or an immunogen for controlled- or sustained-release, as described herein, to the epidermis (e.g., about 10 μm to 120 μm below the skin surface), e.g., to induce an immune response. In some embodiments, the length of microneedles can be fabricated sufficiently long enough to enable delivery of an implantable tip comprising a vaccine, an antigen, and/or an immunogen for controlled- or sustained-release, as described herein, to the dermis (e.g., about 60 μm to about 2.1 mm below the skin surface). An skilled artisan can adjust the microneedle length for a number of factors, including, without limitations, tissue thickness, e.g., skin thickness, (e.g., as a function of age, gender, location on body, species (e.g., animal), drug delivery profile, diffusion properties of the vaccine, antigen, and/or immunogen for controlled- or sustained-release (e.g., the ionic charge and/or molecule weight, and/or shape of the vaccine, antigen, and/or immunogen for controlled- or sustained-release), or any combinations thereof. However, without wishing to be bound by theory, with an approximately 650 μm tall microneedle an implantable sustained-release tip may be deployed at a depth of between about 100 μm and about 600 μm within the dermis layer of the skin to a subject to achieve controlled- or sustained-release of vaccine from the tip. In some embodiments, the microneedle may be about 800 μm tall (e.g., between about 500 μm and 1200 μm tall).

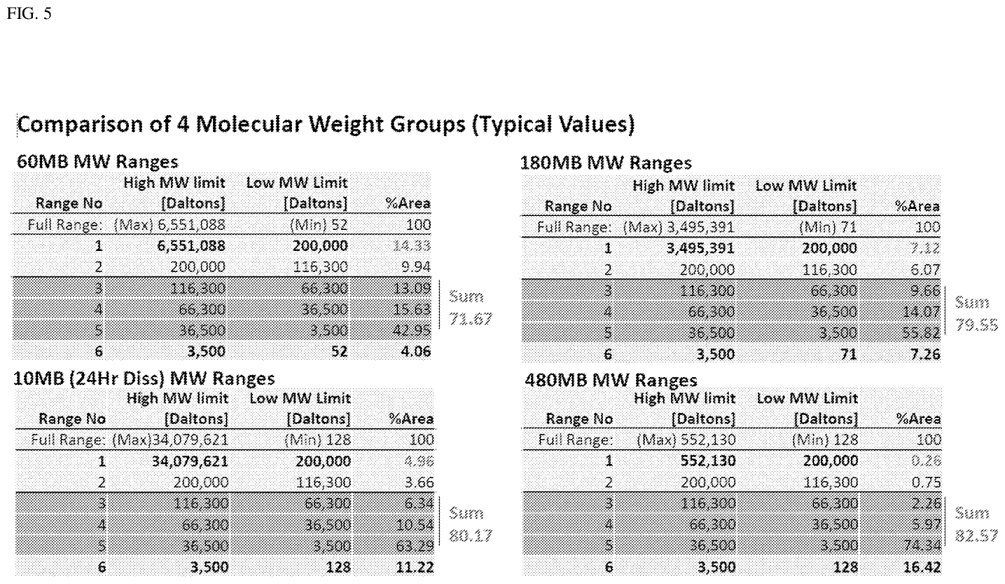

Exemplary microneedles of the invention are depicted in FIGS. 5A-5B.

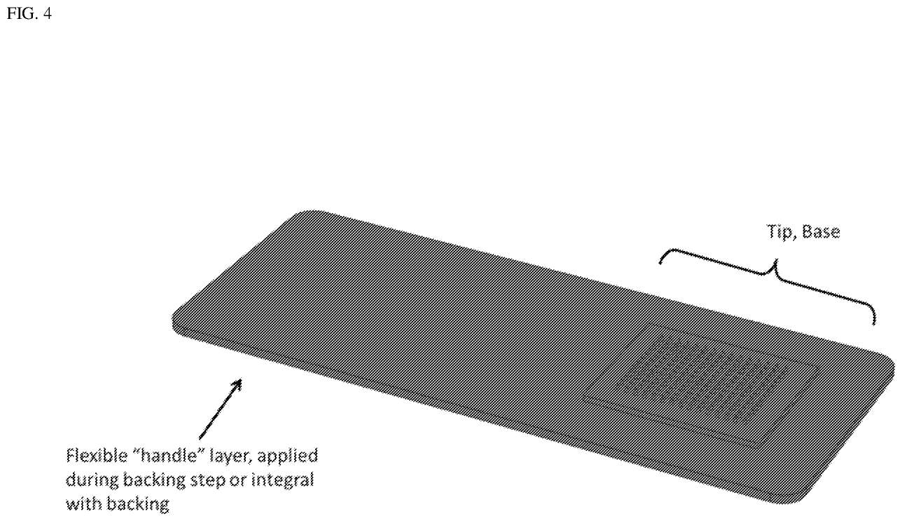

In some embodiments, a plurality of microneedles can be arranged in a random or predefined pattern to form a microneedle array and/or patch, as described herein. The patch may comprise a carrier, backing, or “handle” layer adhered to the back of the base (see, e.g., FIG. 4). This layer can provide structural support and an area by which the patch can be handled and manipulated without disturbing the needle array.

### Microneedle Array

The microneedle array may comprise about 121 needles in an 11×11 square grid with approximately 0.75 mm pitch. Individual needles are cones approximately 0.65 mm long with base diameter approximately 0.35 mm and included angle of approximately 30°. The tip of the needle must be sharp in order to penetrate the skin. The radius of curvature of the tip should ideally be no more than 0.01 mm.

Backing

Exemplary backing materials that can be used in the fabrication of a microneedle of the invention include, but are not limited a solid support, e.g., a paper-based material, a plastic material, a polymeric material, or a polyester-based material (e.g., a Whatman 903 paper, a polymeric tape, a plastic tape, an adhesive-backed polyester tape, or other medical tape). In some embodiments, the backing comprises a Whatman 903 paper. In some embodiments, the backing comprises a polyester tape. In some embodiments, the polyester tape comprises an adhesive-backed polyester tape. In some embodiments, the backing material may be coated (e.g., at least on one side) with an adhesive suitable for bonding to and/or adhering to the dissolvable base of a microneedle described herein.

The backing materials used in the microneedles of the invention may have various properties, including, but not limited to, the ability to bond and/or adhere to the dissolving base layer to permit demolding. A backing material must be strong enough for the backing to maintain patch integrity, e.g., if the dissolving base layer has cracks or discontinuities. The backing material may be sufficiently flexible so as to conform, for example, to a non-flat surface, such as a skin surface. In particular, the backing must be flexible enough during wear time, such as after the patch is applied (e.g., pressed into) the skin. The backing may comprise and/or consist of a non-dissolving material, such that the backing maintains its integrity after patch application to a skin surface and during patch removal from a skin surface.

The backing may have any dimension suitable for application to a target skin surface. In some embodiments, the dimensions of the backing can be a 12 mm diameter circle. In some embodiments, the dimensions of the backing can be a 12 mm wide strip with a “handle” section of up to 12 mm length beyond the edge of the 12 mm×12 mm patch.

Dissolvable Base

The dissolving base layer forms the base of the conical needles (e.g., functions as the support for the distal silk fibroin tips that are loaded with a vaccine, an antigen, and/or an immunogen). The dissolvable base layer can also function as a layer connecting adjacent needles to form a microneedle array or patch. In some embodiments, the dissolvable base layer comprises less than 98% (e.g., less than about 98%, less than about 90%, less than about 80%, less than about 70%, less than about 60%, less than about 50%, less than about less 40%, less than about 30%, less than about 20%, less than about 10%, less than about 9%, less than about 8%, less than about 7%, less than about 6%, less than about 5%, less than about 4%, less than about 3%, less than about 2%, less than about 1%) of the total amount (e.g., dose) of a vaccine, an antigen, and/or an immunogen comprises loaded into the microneedle and/or microneedle device. In some embodiments, the dissolvable base layer does not comprise, e.g., a detectable amount of, a vaccine, an antigen, and/or an immunogen. In some embodiments, dissolvable base layer is formulated to limit and/or reduce the amount of vaccine, antigen, and/or immunogen leakage (e.g., diffusion) from the silk fibroin tips into the dissolvable base layer, e.g., as compared to art known base layer formulations, e.g., base layer formulations comprising PAA. In some embodiments, a limit and/or reduce amount of vaccine, antigen, and/or immunogen leakage (e.g., diffusion) from the silk fibroin tips can be determined about 1 day, about 2 days, about 3 days, about 4 days, about 5 days, or about 6 days; about 1 week, about 2 weeks, or about 3 weeks; about 1 month, about 2 months, about 3 months, about 4 months, about 5 months, about 6 months, about 7 months, about 8 months, about 9 months, about 10 months, or about 11 months; or about 1 year or more after fabrication and storage (e.g., storage at about 4° C. (e.g., refrigeration), at about 25° C. (e.g., room temperature), at about 37° C. (e.g., body temperature), at about 45° C. and/or at about 50° C.), e.g., as compared to a base layer formulation comprising PAA.

The dissolvable base layer comprises a material that can dissolve into the skin, e.g., within the intended wear time (e.g., about five minutes). In some embodiments, the at least about 10%, 20%, 30%, 40%, 50%, 60%, 70%, 80%, 90%, or 100% of the dissolvable base layer is dissolved after application, e.g., to the skin, within the intended wear time (e.g., about 1 minute, about 2 minutes, about 3 minutes, about 4 minutes, about 5 minutes, about 6 minutes, about 7 minutes, about 8 minutes, about 9 minutes, or about 10 minutes or more).

The material used in the fabrication of the dissolvable base must be sufficiently strong enough to enable the microneedle to penetrate the skin, and be tough enough (e.g., not brittle) to also enable demolding. The dissolvable base material must be amenable to routine handling without catastrophic failure, and must retain its mechanical properties between demolding and application (e.g., not so hygroscopic that it melts due to ambient humidity). The dissolvable base layer material must be non-toxic and non-reactogenic at the doses used in a patch. In some embodiments, the dissolvable base layer comprises a water soluble component. In some embodiments, a dissolvable base layer, as described herein, has improved biocompatibility, e.g., as compared to a dissolvable base layer comprising poly(acrylic acid) (PAA). In some embodiments, the dissolvable base layer material causes a reduced inflammatory response and/or reduced tissue necrosis. In some embodiments, the dissolvable base layer material is not PAA, and induces a reduced inflammatory response and/or reduced tissue necrosis compared to PAA. In some embodiments, the dissolvable base layer material has a pH similar to that of the biological barrier into which it will be dissolved, e.g., a pH of about 4.0 to about 8.0

Non-limiting examples of materials that may be used to fabricate the dissolvable base layer include gelatin (e.g., hydrolyzed gelatin), polyethylene glycol (PEG), sucrose, low-viscosity carboxymethylcellulose (CMC), polyvinylpyrrolidone (PVP), polyvinyl alcohol (PVA), hyaluronate, maltose, and/or methyl cellulose. In some embodiments, the dissolvable base comprises one, two, three, four, five, six, seven, eight, or more (e.g., all) of gelatin, polyethylene glycol (PEG), sucrose, carboxymethylcellulose (CMC), polyvinylpyrrolidone (PVP), polyvinyl alcohol (PVA), hyaluronate, maltose, and methyl cellulose, e.g., at a concentration between about 1% and about 75% (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75%). In some embodiments, the dissolvable base does not comprise a therapeutic agent, as described herein.

In some embodiments, the dissolvable base comprises between about 10% and about 70% gelatin (e.g., hydrolyzed gelatin) (e.g., about 10%, about 20%, about 30%, about 40%, about 50%, about 60%, or about 70% gelatin).

In some embodiments, the dissolvable base comprises between about 1% and about 70% polyethylene glycol (PEG) (e.g., about 1%, about 2%, about 3%, about 4%, about 5%, about 6%, about 7%, about 8%, about 9%, about 10%, about 20%, about 30%, about 40%, about 50%, about 60%, or about 70% PEG).

In some embodiments, the dissolvable base comprises between about 1% and about 35% sucrose (e.g., about 1%, about 2%, about 3%, about 4%, about 5%, about 6%, about 7%, about 8%, about 9%, about 10%, about 15%, about 20%, about 25%, about 30%, or about 35% sucrose).

In some embodiments, the dissolvable base comprises between about 1% and about 35% CMC (e.g., about 1%, about 2%, about 3%, about 4%, about 5%, about 6%, about 7%, about 8%, about 9%, about 10%, about 15%, about 20%, about 25%, about 30%, or about 35% CMC).

In some embodiments, the dissolvable base comprises between about 10% and about 70% PVP (e.g., about 10%, about 20%, about 30%, about 40%, about 50%, about 60%, or about 70% PVP).

In some embodiments, the dissolvable base comprises between about 1% and about 35% PVA (e.g., e.g., about 1%, about 2%, about 3%, about 4%, about 5%, about 6%, about 7%, about 8%, about 9%, about 10%, about 15%, about 20%, about 25%, about 30%, or about 35% PVA).

In some embodiments, the dissolvable base comprises between about 1% and about 75% (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% hyaluronate). In some embodiments, the dissolvable base comprises between about 1% and about 75% (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% maltose).

In some embodiments, the dissolvable base comprises between about 1% and about 75% (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% methyl cellulose).

In some embodiments, the dissolvable base layer may comprise 40% hydrolyzed gelatin, 10% Sucrose w/v in DI water. Optionally, the base layer may include 1% low-viscosity carboxymethylcellulose (CMC), which may reduce brittleness. In some embodiments, the dissolvable base layer may comprise polyvinylpyrrolidone (PVP) of 10 kD MW at up to 50% w/v in DI water; polyvinyl alcohol (PVA) 87% hydrolyzed at 13 kD MW at up to 20% in DI water; or CMC at up to 10% in DI water. The following combinations may also be suitable for use in the fabrication of a dissolvable base layer: 30% PVP and 10% PVA; 37% PVP, 5% PVA, and 15% sucrose; or various other proportions of PVP, PVA, and sucrose.

In some embodiments, the dissolvable base layer is approximately 12 mm square and 0.75 mm thick. In some embodiments, the dissolvable base layer can cover the entire patch. In some embodiments, the dimension of the base layer can be a 12 mm diameter circle, or a 12×12 mm square.

Implantable Sustained-Release Tip

In embodiments, the implantable sustained-release tip can be fabricated from silk fibroin and may comprise a vaccine, an antigen, and/or an immunogen as described herein (e.g., an influenza vaccine). In some embodiments, the implantable sustained-release tip can be designed to be deployed into the dermis layer of the skin (e.g., not into the subcutaneous space), as the population of professional antigen presenting cells in the dermis is much higher than in the subcutaneous space. In humans, the dermis ranges from about 1000-2000 μm (e.g., about 1-2 mm) thick based on location and patient age and health. In rodents, the dermis is much thinner (e.g., mice ˜100-300 μm, and rats ˜800-1200 μm). Without wishing to be bound by theory, with a 650 μm tall microneedle an implantable sustained-release tip may be deployed at a depth of between about 100 μm and about 600 μm to achieve the controlled- or sustained-release of a vaccine, an antigen, and/or an immunogen as described herein (e.g., an influenza vaccine).

Without being bound by theory, the molecular weight of the silk fibroin solution used in the fabrication of a microneedle described herein can function as a control factor to modulate the controlled- or sustained-release of a vaccine, an antigen, and/or an immunogen (e.g., an influenza vaccine) from the tip. In some embodiments, a higher molecular weight silk fibroin solutions can favor a slower controlled- or sustained-release (e.g., reducing the amount of an initial burst (e.g., the amount released on Day 0) by at least about 10% and then releasing additional antigen over at least about the next 4 days). In some embodiments, the controlled- or sustained-release of a vaccine, an antigen, and/or an immunogen (e.g., an influenza vaccine) from the tip may be over at least about 4 days (e.g., about 4, 5, 6, 7, 8, 9, 10, 11, 12, 13, or 14 or more days, e.g., between about 4 days and about 14 days, e.g., between about 1-2 weeks, about 1-3 weeks, or about 1-4 weeks). In some embodiments, controlled- or sustained-release occurs over about 1 week to about 2 weeks.

In embodiments, the silk fibroin solution used in the fabrication of a microneedle described herein can be a low molecular weight silk fibroin composition comprising a population of silk fibroin fragments having a range of molecular weights, characterized in that: no more than 15% of the total number of silk fibroin fragments in the population has a molecular weight exceeding 200 kDa, and at least 50% of the total number of the silk fibroin fragments in the population has a molecular weight within a specified range, wherein the specified range is between about 3.5 kDa and about 120 kDa, or between about 5 kDa and about 125 kDa. Stated another way, the silk fibroin solution used in the fabrication of a microneedle described herein can comprise a population of silk fibroin fragments having a range of molecular weights, characterized in that: no more than 15% of the total moles of silk fibroin fragments in the population has a molecular weight exceeding 200 kDa, and at least 50% of the total moles of the silk fibroin fragments in the population has a molecular weight within a specified range, wherein the specified range is between about 3.5 kDa and about 120 kDa, or between about 5 kDa and about 125 kDa. (see, e.g., WO2014/145002, incorporated herein by reference herein).

Exemplary silk fibroin (e.g., regenerated silk fibroin) solutions may have different molecular weight profiles are shown as determined by size exclusion chromatography (SEC) methods (see, e.g., FIG. 5). In some embodiments, the silk fibroin solutions can be prepared, e.g., according to established methods. In some embodiments, pieces of cocoons from the silkworm Bombyx mori were first boiled in 0.02 M Na2CO3 to remove sericin protein which is present in unprocessed, natural silk, prior to analysis by SEC. In some embodiments, silk fibroin composition can be a composition or mixture produced by degumming cocoons from the silkworm Bombyx mori at an atmospheric boiling temperature for about 480 minutes or less, e.g., less than 480 minutes, less than 400 minutes, less than 300 minutes, less than 200 minutes, less than 180 minutes, less than 120 minutes, less than 100 minutes, less than 60 minutes, less than 50 minutes, less than 40 minutes, less than 30 minutes, less than 20 minutes, less than 10 minutes or shorter. In one embodiment, the silk fibroin composition can be a composition or mixture produced by degumming silk cocoon at an atmospheric boiling temperature in an aqueous sodium carbonate solution for about 480 minutes or less, e.g., less than 480 minutes, less than 400 minutes, less than 300 minutes, less than 200 minutes, less than 180 minutes, less than 120 minutes, less than 100 minutes, less than 60 minutes, less than 50 minutes, less than 40 minutes, less than 30 minutes, less than 20 minutes, less than 10 minutes or shorter.

In some embodiments, the silk fibroin solution may be a 10-minute boil (10 MB), a 60-minute boil (60 MB), a 120-minute boil (120 MB), a 180-minute boil (180 MB), or a 480-minute boil (480 MB) silk fibroin solution (see, e.g., FIG. 5). In some embodiments, an influenza vaccine, antigen, and/or immunogen can be formulated in a 1% w/v to about 10% w/v (e.g., about 1, 2, 3, 4, 5, 6, 7, 8, 9, or 10% w/v) 10 MB silk fibroin solution. In some embodiments, an influenza vaccine, antigen, and/or immunogen can be formulated in a 1% w/v to about 10% w/v (e.g., about 1, 2, 3, 4, 5, 6, 7, 8, 9, or 10% w/v) 60 MB silk fibroin solution. In some embodiments, an influenza vaccine, antigen, and/or immunogen can be formulated in a 1% w/v to about 10% w/v (e.g., about 1, 2, 3, 4, 5, 6, 7, 8, 9, or 10% w/v) 120 MB silk fibroin solution. In some embodiments, an influenza vaccine, antigen, and/or immunogen can be formulated in a 1% w/v to about 10% w/v (e.g., about 1, 2, 3, 4, 5, 6, 7, 8, 9, or 10% w/v) 180 MB silk fibroin solution. In some embodiments, an influenza vaccine, antigen, and/or immunogen can be formulated in a 1% w/v to about 10% w/v (e.g., about 1, 2, 3, 4, 5, 6, 7, 8, 9, or 10% w/v) 480 MB silk fibroin solution.

Without being bound by theory, the primary tunability of the implantable sustained-release tip is its crystallinity, measured via beta-sheet content (intermolecular and intramolecular β-sheet). This impacts the solubility of the silk tip matrix and the ability of antigen to be retained. With the increased β-sheet content, the tip also becomes more mechanically strong. Specific vaccine release profiles are achieved through modulation of the crystallinity and the diffusivity of the silk matrix. This is accomplished through both silk input material and formulation as well as post-treatment to increase crystallinity (e.g. water annealing, methanol/solvent annealing). In some embodiments, the implantable controlled- or sustained-release microneedle tip comprises a beta-sheet content of between about 10% and about 60% (e.g., about 10%, about 20%, about 30%, about 40%, about 50%, about 60%), e.g., as based on a “crystallinity index,” e.g., a “crystallinity index” known in the art. In some embodiments, the implantable controlled- or sustained-release microneedle tip can be formulated as a particle (e.g., a microparticle and/or a nanoparticle).

Dimensions of the Implantable Sustained-Release Tip

The methods provided herein can be used to fabricate silk fibroin-based implantable sustained-release tips of any dimensions, e.g., ranging from about 75 μm to about 800 μm in height/length (e.g., about 75, about 100 μm, about 125 μm, about 150 μm, about 250 μm to about 300 μm, about 300 μm to about 350 μm, about 350 μm to about 400 μm, about 400 μm to about 450 μm, about 450 μm to about 500 μm, about 500 μm to about 550 μm, about 550 μm to about 600 μm, about 600 μm to about 650 μm, about 650 μm to about 700 μm, about 700 μm to about 750 μm, about 750 μm, to about 800 μm), and/or having a tip radius of about 10 μm or less (e.g., between about 1 μm and about 10 μm, e.g., about 1 μm or less, about 2 μm or less, about 3 μm or less, about 4 μm or less, about 5 μm or less, about 6 μm or less, about 7 μm or less, about 8 μm or less, about 9 μm or less, or about 10 μm or less). In some embodiments, the implantable tip can have a diameter of any size, e.g., based upon the type of biological barrier (e.g., skin layer) intended to be pierced by the tip. In embodiments, the tip can have a dimension (e.g., a diameter) ranging from about 50 nm to about 50 μm (e.g., about 50 nm to about 250 nm, about 250 nm to about 500 nm, about 500 to about 750 nm, about 750 nm to about 1 μm, about 1 μm to about 5 μm, about 5 μm to about 10 μm, about 10 μm to about 15 μm, about 15 μm to about 20 μm, about 20 μm to about 25 μm, about 25 μm to about 30 μm, about 30 μm to about 35 μm, about 35 μm to about 40 μm, about 40 μm to about 45 μm, or about 45 μm to about 50 μm). It can be understood that there is no fundamental limitation preventing the sustained-release tips from having even smaller diameters (e.g., the limit of silk replica casting has been demonstrated with a resolution of tens of nm, see, e.g., Perry et al., 20 Adv. Mat. 3070 (2008)).

In some embodiments, the sharpness of the implantable sustained-release tip point is described herein in terms of tip radius. The molds used in the fabrication of the microneedles described herein are designed to have a tip radius between about 0.5 μm to about 10 μm (e.g., about 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, or 10 μm). In some embodiments, the tip radius is between about 20 μm to about 25 μm (e.g., about 20 μm, 21 μm, 22 μm, 23 μm, 24 μm, or 25 μm). Without being bound by theory, it can be understood that blunter needles may require more force to penetrate the epidermis. In embodiments, other dimensions of the implantable sustained-release tip may be controlled by the shape of the mold and fill volume. In some embodiments, the implantable sustained-release tip have an included angle between about 5 degrees and about 45 degrees (e.g., about 5, 6, 7, 8, 9, 10, 15, 20, 25, 30, 35, 40, or 45 degrees). In some embodiments, the implantable sustained-release tip can have an included angle between about 15 degrees and 45 degrees (e.g., about 15 degrees, about 16 degrees, about 17 degrees, about 18 degrees, about 19 degrees, about 20 degrees, about 21 degrees, about 22 degrees, about 23 degrees, about 24 degrees, about 25 degrees, about 26 degrees, about 27 degrees, about 28 degrees, about 29 degrees, about 30 degrees, about 31 degrees, about 32 degrees, about 33 degrees, about 34 degrees, about 35 degrees, about 36 degrees, about 37 degrees, about 38 degrees, about 39 degrees, about 40 degrees, about 41 degrees, about 42 degrees, about 43 degrees, about 44 degrees, or about 45 degrees.

In embodiments, the height of the implantable sustained-release tip may depend on the formulation and print volume, which can influence the surface tension and drying kinetics. In some embodiments, the height of the implantable sustained-release tip may extend to half of the full height of the microneedle. In some embodiments, the height of the implantable sustained-release tip is between about 75 μm to about 475 μm (e.g., about 75, about 100 μm, about 125 μm, about 150 μm, about 175 μm, about 200 μm, about 225 μm, about 250 μm, about 275 μm, about 300 μm, about 325 μm, about 375 μm, about 400 μm, about 425 μm, or about 475 μm). In some embodiments, the base of the tip comprises a thin “shell”-like layer roughly between about 5-10 μm thick (e.g., about 5, 6, 7, 8, 9, or 10 μm thick). In some embodiments, the implantable sustained-release tip may dry to a more solid construct with minimal “shell” wherein the height may be closer to 150 μm (e.g., between about 50 μm and about 200 μm) and the thickness >50 μm (e.g., between about 25 μm and about 75 μm).

Further, the microneedles of the present invention can take advantage of art known techniques developed, e.g., to functionalize silk fibroin (e.g., active agents such as dyes and sensors). See, e.g., U.S. Pat. No. 6,287,340, Bioengineered anterior cruciate ligament; WO 2004/000915, Silk Biomaterials & Methods of Use Thereof; WO 2004/001103, Silk Biomaterials & Methods of Use Thereof; WO 2004/062697, Silk Fibroin Materials & Use Thereof; WO 2005/000483, Method for Forming inorganic Coatings; WO 2005/012606, Concentrated Aqueous Silk Fibroin Solution & Use Thereof; WO 20111005381, Vortex-Induced Silk fibroin Gelation for Encapsulation & Delivery; WO 20051123114, Silk-Based Drug Delivery System; WO 2006/076711, Fibrous Protein Fusions & Uses Thereof in the Formation of Advanced Organic/Inorganic Composite Materials; U.S. Application Pub. No. 2007/0212730, Covalently immobilized protein gradients in three-dimensional porous scaffolds; WO 2006/042287, Method for Producing Biomaterial Scaffolds; WO 2007/016524, Method for Stepwise Deposition of Silk Fibroin Coatings; WO 2008/085904, Biodegradable Electronic Devices; WO 20081118133, Silk Microspheres for Encapsulation & Controlled Release; WO 20081108838, Microfluidic Devices & Methods for Fabricating Same; WO 20081127404, Nanopatterned Biopolymer Device & Method of Manufacturing Same; WO 20081118211, Biopolymer Photonic Crystals & Method of Manufacturing Same; WO 20081127402, Biopolymer Sensor & Method of Manufacturing Same; WO 20081127403, Biopolymer Optofluidic Device & Method of Manufacturing the Same; WO 20081127401, Biopolymer Optical Wave Guide & Method of Manufacturing Same; WO 20081140562, Biopolymer Sensor & Method of Manufacturing Same; WO 20081127405, Microfluidic Device with Cylindrical Microchannel & Method for Fabricating Same; WO 20081106485, Tissue-Engineered Silk Organs; WO 20081140562, Electroactive Bioploymer Optical & Electro-Optical Devices & Method of Manufacturing Same; WO 20081150861, Method for Silk Fibroin Gelation Using Sonication; WO 20071103442, Biocompatible Scaffolds & Adipose-Derived Stem Cells; WO 20091155397, Edible Holographic Silk Products; WO 20091100280, 3-Dimensional Silk Hydroxyapatite Compositions; WO 2009/061823, Fabrication of Silk Fibroin Photonic Structures by Nanocontact Imprinting; WO 20091126689, System & Method for Making Biomaterial Structures.

In various embodiments, the silk fibroin-based microneedle tips can further comprise at least one additional therapeutic agent, wherein the additional therapeutic can be dispersed throughout the microneedle or form at least a portion of the microneedle tip. In some embodiments, the additional therapeutic agent is useful in the treatment of a viral infection described herein. Optionally the silk fibroin-based microneedle tips can further comprise an excipient and/or adjuvant, as described herein.

Methods of Making and/or Manufacturing a Microneedle

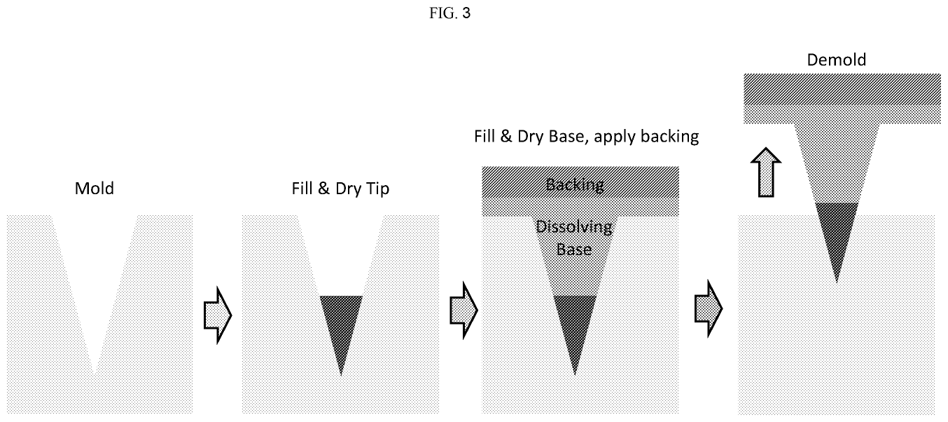

A schematic diagram and a flow chart depicting the method of fabrication of a microneedle of the invention are shown in FIGS. 3 and 4, respectively. Machine vision guided printing of precise nL volumes of silk fibroin solution into individual needle cavities enables different dosages and formulations to be incorporated within releasable tips of a microneedle device (e.g., a microneedle array or patch). An exemplary microneedle device (e.g., a microneedle array or patch), comprises an 11×11 cone array. It should be understood that the microneedle device may include needle cavities produced in an array of varying number of cavities and orientations to achieve a desired result.

Mold Production

In some embodiments, a mold is used in the fabrication of a microneedle device. As will be discussed in greater detail below, a sterilized mold is used to produce a microneedle device having an array of releasable tips embodying an antigen-silk formulation.

For example, a silicone (DOW Corning Sylgard® 184) resin may be cast against a positive master having the intended geometry of a microneedle array. Once the silicone has cured, it may be removed from the master. The master can then be reused for a large number of silicone castings. Throughout the fabrication process the silicone mold may be inspected for defects (e.g., between castings). If desired, the silicone mold can be sterilized, for example, by autoclaving. In one embodiment, the mold includes a mold body having an array of needle cavities formed within the mold body.

In some embodiments, other types of silicone and/or other materials and processes may be used to fabricate the mold. For example, liquid silicone injection molding and thermoplastic elastomer injection molding may be used. Without wishing to be bound by theory, it may be understood that a key requirement is that the mold material be soft and flexible (e.g., comprise a Shore hardness of about 50 A) and have low adhesion with silk and other materials used in the construction of the patch.

Tip Filling

Tip formulation consisting of silk fibroin, antigen, and potentially other excipients in aqueous solution, is dispensed into each needle cavity in the mold via nanoliter printing. Currently this is done at lab scale using a Biojet Elite™ AD3400 dispensing system produced by BioDot, but systems with similar capabilities made by other suppliers can be employed. The working volume of the BioDot™ dispenser is enclosed and is maintained at 60% relative humidity (RH) to slow drying of the formulation and avoid buildup of dry solids on the dispensing nozzle.

Molds are placed within a fixture that constrains their locations on the processing platform of the BioDot™ dispenser. The machine uses a camera to image each mold and a machine vision algorithm identifies the precise location and orientation of the array of needle cavities in each mold. This location is used to direct the subsequent dispensing steps. The filled molds are inspected using a stereomicroscope for filling defects such as misaligned dispenses or large bubbles in the liquid.

Primary Drying

The filled molds are set aside to dry within the machine enclosure for about 7 minutes. After drying, the above dispensing process is repeated and the molds are dried again for 7 minutes. This is the “primary” drying step.

Secondary Drying

The molds are moved to a chamber in which humidity is controlled to about 25% RH and ambient room temperature and kept overnight (about 14 hours) to complete drying. This is the “secondary” drying step.

Water Annealing

The molds are transferred to a vacuum desiccator that also contains about 500 mL of DIW. The desiccator is closed and vacuum is drawn for about 5 minutes using the main vacuum line in the lab. After 5 minutes, the outlet valve of the desiccator is closed and it is placed within an incubator holding at 37° C. for four hours. After four hours, the desiccator is vented and the molds are transferred back to the 25% RH chamber at ambient room temperature.

Post-Anneal Drying

Molds are kept at 25% RH for at least four hours or up to overnight before subsequent steps.

Base Layer Filling

The dissolvable base layer is formed by filling the mold with a solution of 40% w/v Hydrolyzed Gelatin and 10% w/v Sucrose in DIW and then drying this layer. First, 150 μL of base solution is spread evenly over the mold with a pipette. Next, the molds are centrifuged at 3900 rpm for 2 minutes. The molds are inspected and if any needle cavities remain unfilled, the filling and centrifuging process is repeated. The molds are “topped off” with 50 μL of base solution.

Base Drying

The filled molds are transferred back to the chamber at 25% RH and dried at least overnight and up to 3 days.

Backing Application

The patches used to generate the release, e.g., controlled- or sustained-release, and improved immunogenicity (see, e.g., the Examples) had a paper backing layer; however, subsequent development has shown that adhesive plastic tape has superior performance as a backing layer.

The paper backing process is as follows: the dried base layer is partially re-wetted with 10-30 μL of DIW spread over the surface with a pipette. Whatman 903 paper is punched into 12 mm diameter circles. The circles of paper are gently pressed into the wet surface of the base layer. The wet base layer partially soaks into the paper. The molds with backing are transferred back into the 25% RH chamber to dry for at least 4 hours until use.

Adhesive Tape Process

Adhesive-backed polyester tape (e.g., 3M® Magic™ tape) is cut into a piece about 12 mm wide and about 25 mm long. One end of the tape is aligned with the patch and gently pressed onto the surface of the base layer. The free end of the tape is folder over onto itself to form a non-adhesive “handle.”

Demolding

The patches are removed from the mold before use. The flexible mold is gently bent away from the stiffer patch, and the patch is taken away from the mold. The patch is inspected for defects such as missing or broken needles.

Packaging

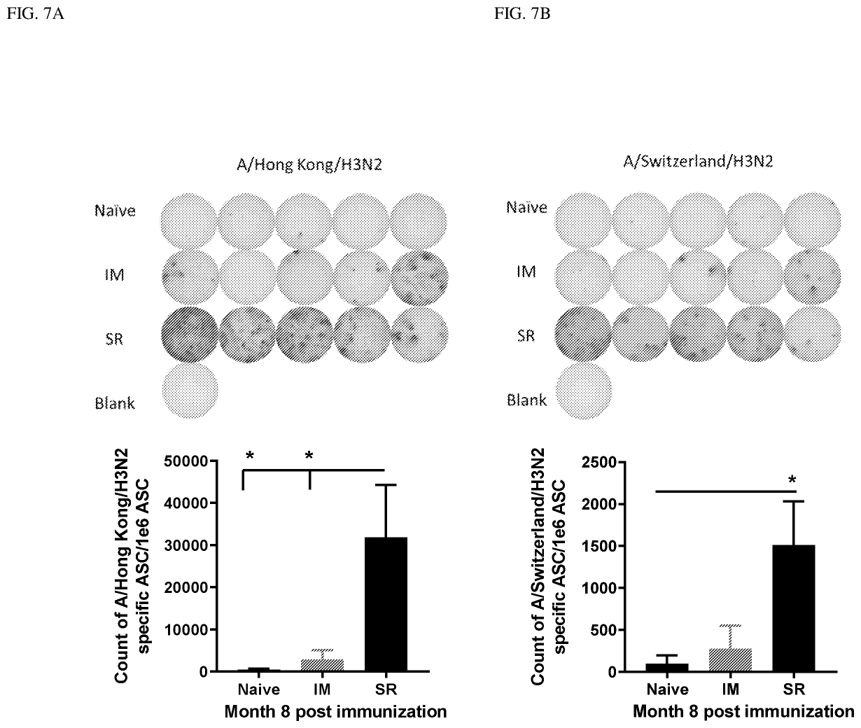

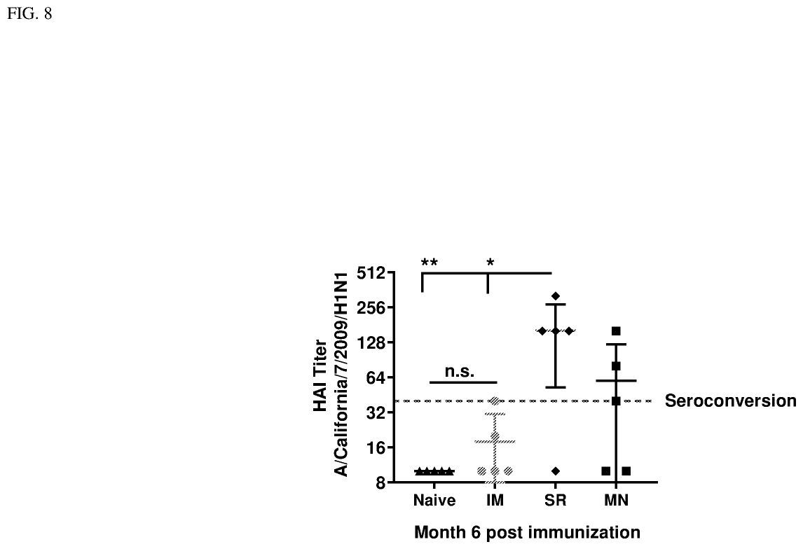

In the studies above, the patches were used soon after demolding and were not packaged. If extended storage is needed, assembled patches can be packaged in a container with low moisture vapor transmission rate (e.g., glass vial or thermoformed plastic tray made of low MVTR materials and a foil-backed heat-sealed lid) along with a desiccant to maintain about rate between about 0% and about 50% (e.g., between about 0% and 10%, between about 10% and about 20%, between about 20% and about 30%, between about 30% and about 40%, or between about 40% and 50%, e.g., about 25%) relative humidity inside the package (see, e.g., FIG. 7).

### Viruses, Antigens, and Immunogens

The present invention provides, in some embodiments, the delivery, e.g., the controlled- or sustained-delivery, of various therapeutic agents, such as vaccines, antigens, and/or immunogens derived from a virus that is a member of the family Orthomyxovirus, e.g., by a formulation, composition, articles, device, preparations, microneedle and/or microneedle device (e.g., a microneedle patch) described herein and/or according to a method described herein. In some embodiments, a vaccine, a microneedle, and/or a microneedle device (e.g., a microneedle patch) described herein may comprise a negative-sense ssRNA virus and/or an RNA virus, such as an influenza virus. In some embodiments, the vaccine, antigen, and/or immunogen comprises a nucleic acid (e.g., a DNA and/or RNA) derived from an influenza virus. In some embodiments, the vaccine, antigen, and/or immunogen comprises an amino acid (e.g., a peptide and/or protein) derived from an influenza virus. In some embodiments, the influenza vaccine, antigen, and/or immunogen comprise an inactivated and/or a live attenuated virion, or split virion, of an influenza virus. In some embodiments, the vaccine and/or the microneedle comprises a non-replicating viral antigen.

In particular, the invention contemplates a vaccine, a microneedle, and/or a microneedle device (e.g., a microneedle patch) comprising an influenza virus vaccine, antigen, and/or immunogen. The influenza virus is a RNA virus (e.g., a linear negative-sense single stranded RNA virus). There are four known genera of influenza virus, each containing a single type (e.g., Influenza A, B, C, and D). Influenza viruses can continuously change and are subject to both antigenic drift and antigenic shift. Exemplary influenza strains are further described in the Examples (see, e.g., Tables 1 and 2).

Influenza A can be divided into subtypes on the basis of two proteins on the surface of the virus: hemagglutinin (HA) and neuraminidase (NA). Influenza A comprises 18 known HA subtypes, referred to herein as H1-H18, and 11 known NA subtypes, referred to herein as N1-N11. Many different combinations of HA and NA proteins may be found on the surface of the influenza A virus. For example, an “H1N1 virus” designates an influenza A virus subtype comprising an H1 protein and an N1 protein. Exemplary influenza A virus subtypes confirmed to infect humans include, but are not limited to, H1N1, H3N2, H2N2, H5N1, H7N7, H1N2, H9N2, H7N2, H7N3, H10N7, and H7N9. The H1N1 virus and H3N2 virus are currently in general circulation among humans.

Exemplary Influenza B viruses may belong to, e.g., the B/Yamagata lineage and/or the B/Victoria lineage.

Vaccines

Non-limiting examples of influenza vaccines for use in the microneedles and microneedle devices (e.g., microneedle patches) described herein can include a commercial vaccine, such as a seasonal vaccine, a pandemic vaccine, and/or a universal vaccine; egg-based vaccines, cell-culture based vaccines; recombinant vaccines; live attenuated, inactivated whole virus, split virion, and/or protein subunit vaccines; and adjuvanted vaccines. Various commercial influenza vaccines are listed below. Additionally, influenza vaccines comprising an mRNA, a DNA, a viral vector, and/or a virus-like particle (VLP) are suitable for use in the microneedles and microneedle devices (e.g., microneedle patches) described herein. In some embodiments, the influenza vaccine may target matrix protein 1, matrix protein 2 (M2e), and/or nucleoprotein (NP) of an influenza virus.

| | |
|---|---|
| Vaccine | Manufacturer |

Seasonal Influenza Vaccines

| | |
|---|---|
| Fluzone High Dose | Sanofi Pasteur |
| Fluzone Quadrivalent | Sanofi Pasteur |
| Fluzone Intradermal Quadrivalent | Sanofi Pasteur |
| Afluria/Fluvax | Seqirus |
| Agriflu | Seqirus |
| Fluad | Seqirus |
| Flucelvax | Seqirus |
| Fluvirin | Seqirus |
| Aggripal | Seqirus |
| FluMist Quadrivalent | MedImmune |
| Flublok | Protein Sciences (Sanofi Pasteur) |
| FluLaval | GlaxoSmithKline |
| Fluarix | GlaxoSmithKline |
| Influvac | Mylan |
| Preflucel | Nanotherapeutics |
| Anflu | Sinovac Biotech |

Pandemic Influenza Vaccines

| | |
|---|---|
| Influenza Virus Vaccine, H5N1 | Sanofi Pasteur |
| Pandemrix | GlaxoSmithKline |
| Panflu | Sinovac Biotech |
| Panflu 1 | Sinovac Biotech |

### Vaccine Formulations and Composition for Controlled- or Sustained-Release

At least one vaccine, antigen, and/or immunogen described herein (e.g., at least one vaccine, antigen, and/or immunogen derived from an influenza virus described herein) can be incorporated into a variety of formulations, compositions, articles, devices, and/or preparations for administration, e.g., to achieve controlled- and/or sustained release. More particularly, at least one vaccine, antigen, and/or immunogen described herein (e.g., at least one vaccine, antigen, and/or immunogen derived from an influenza virus described herein) can be formulated into formulations, compositions, articles, devices, and/or preparations by combination with appropriate, pharmaceutically acceptable carriers or diluents, and can be formulated into preparations in semi-solid, solid, or liquid formats. In some embodiments, the formulations, compositions, articles, devices, and/or preparations described herein comprise silk fibroin. Exemplary formulations, compositions, articles, devices, and/or preparations comprise: a microneedle (e.g., a microneedle device, e.g., a microneedle patch, e.g., as described herein), an implantable device (e.g., a pump, e.g., a subcutaneous pump), an injectable formulation, a depot, a gel (e.g., a hydrogel), an implant, and a particle (e.g., a microparticle and/or a nanoparticle). As such, administration of the compositions can be achieved in various ways, including intradermal, intramuscular, transdermal, subcutaneous, or intravenous administration. Moreover, the formulations, compositions, articles, devices, and/or preparations can be formulated and/or administered to achieve controlled- and/or sustained release of the at least one vaccine, antigen, and/or immunogen described herein (e.g., at least one vaccine, antigen, and/or immunogen derived from an influenza virus described herein).

In some embodiment, the vaccine (e.g., the influenza vaccine) is administered, e.g., substantially sustained, over a period of, or at least 1, 5, 10, 15, 30, 45 minutes; a period of, or at least, 1, 2, 3, 4, 5, 10, 24 hours; a period of, or at least, 1, 2, 3, 4, 5, 6, 7, 8, 9, 10, 11, 12, 13, 14 days; a period of, or at least, 1, 2, 3, 4, 5, 6, 7, 8 weeks; a period of, or at least 1, 2, 3, 4, 5, 6, 7, 8, 9, 10, 11 months; a period of, or at least, 1, 2, 3, 4, 5 years, or longer. In one embodiment, the vaccine (e.g., the influenza vaccine) is administered as a controlled- or sustained release formulation, dosage form, or device. In certain embodiments, the vaccine (e.g., the influenza vaccine) is formulated for continuous delivery, e.g., intradermal, intramuscular, and/or intravenous continuous delivery. In some embodiments, the composition or device for the controlled- or sustained-release of the vaccine is chosen from: a microneedle (e.g., a microneedle device, e.g., a microneedle patch), an implantable device (e.g., a pump, e.g., a subcutaneous pump), an injectable formulation, a depot, a gel (e.g., a hydrogel), an implant, or a particle (e.g., a microparticle and/or a nanoparticle). In one embodiment, the vaccine (e.g., the influenza vaccine) is in a silk-based controlled- or extended release dosage form or formulation (e.g., a microneedle described herein). In one embodiment, the vaccine (e.g., the influenza vaccine) is administered via an implantable device, e.g., a pump (e.g., a subcutaneous pump), an implant, an implantable tip of a microneedle, or a depot. The delivery method can be optimized such that a vaccine (e.g., an influenza vaccine) dose as described herein (e.g., a standard dose) is administered and/or maintained in the subject for a pre-determined period (e.g., a period of, or at least: 1, 5, 10, 15, 30, 45 minutes; 1, 2, 3, 4, 5, 10, 24 hours 1, 2, 3, 4, 5, 6, 7, 8, 9, 10, 11, 12, 13, 14 days; 1, 2, 3, 4, 5, 6, 7, 8 weeks; 1, 2, 3, 4, 5, 6, 7, 8, 9, 10, 11 months; 1, 2, 3, 4, 5 years, or longer). The substantially sustained or extended release of the vaccine (e.g., the influenza vaccine) can be used for prevention or treatment of a viral infection (e.g., an influenza viral infection) for a period of hours, days, weeks, months, or years.

The present invention provides, in some embodiments, formulations, compositions, articles, devices, and/or preparations of the invention can be formulated and/or configured for controlled- or sustained-release of a at least one vaccine, antigen, and/or immunogen (e.g., at least one vaccine, antigen, and/or immunogen derived from an influenza virus described herein) in an amount (e.g., a dosage) and/or over a time period sufficient to result in an immune response (e.g., a cellular immune response and/or a humoral immune response) to the virus, e.g., the influenza virus, in the subject.

In some embodiments, the formulations, compositions, articles, devices, and/or preparations of the invention can be formulated and/or configured for controlled- or sustained-release of a at least one vaccine, antigen, and/or immunogen (e.g., at least one vaccine, antigen, and/or immunogen derived from an influenza virus described herein) in an amount (e.g., a dosage) and/or over a time period sufficient to result in broad spectrum immunity in the subject.

The substantially continuously or extended release delivery or formulation of the vaccine (e.g., the influenza vaccine) can be used for prevention or treatment of a viral infection (e.g., an influenza viral infection) for a period of hours, days, weeks, months, or years.

In some embodiments, at least one vaccine, antigen, and/or immunogen described herein can be added to the silk fibroin solution, e.g., before forming the silk fibroin microneedles or microneedle devices described herein. In embodiments, a silk fibroin solution can be mixed with a vaccine, antigen, and/or immunogen, and then used in the fabrication of an implantable microneedle tip, e.g., by the process of filling and/or casting, drying, and/or annealing to produce a microneedle having any of the desired material properties, as described herein.

Without being bound by theory, the ratio of silk fibroin to vaccine, antigen, and/or immunogen in an implantable tip of a microneedle influences their release. In some embodiments, increased silk concentration in the implantable tip favors a slower release and/or greater antigen retention within the tip. Any concentration of silk may be used, as long as the concentration allows for printing and has the mechanical strength sufficient to pierce the skin.

In some embodiments, silk fibroin can be used at a concentration ranging from about 1% w/v to about 10% w/v (e.g., about 1, 2, 3, 4, 5, 6, 7, 8, 9, or 10% w/v) in the fabrication of a microneedle, or a component thereof, as described herein.

Exemplary Excipients

In addition, the formulations, compositions, articles, devices, and/or preparations can be formulated with common excipients, diluents or carriers for administered by the intradermal, intramuscular, transdermal, subcutaneous, or intravenous routes. In some embodiments, the formulations, compositions, articles, devices, and/or preparations can be administered, e.g., transdermally, and can be formulated as controlled- or sustained-release dosage forms and the like. The formulations, compositions, articles, devices, and/or preparations described herein can be administered alone, in combination with each other, or they can be used in combination with other known therapeutic agents.

Suitable formulations for use in the present invention are found in Remington's Pharmaceutical Sciences (1985). Moreover, for a review of methods for drug delivery, see, Langer (1990) Science 249:1527-1533. The formulations, compositions, articles, devices, and/or preparations described herein can be manufactured in a manner that is known to those of skill in the art, e.g., by mixing, dissolving, granulating, dragee-making, levigating, emulsifying, encapsulating, entrapping or lyophilizing processes. The following methods and excipients are merely exemplary and are in no way limiting.

The silk fibroin formulations used in the fabrication of the microneedles described herein may include excipients. In embodiments, inclusion of an excipient may be for the purposes of improving the stability of an incorporated vaccine, antigen, and/or immunogen; to increase silk matrix porosity and diffusivity of the vaccine, antigen, and/or immunogen from the formulation, composition, article, device, preparation, and/or microneedle, e.g., microneedle tip; and/or to increase crystallinity/beta-sheet content of silk matrix to render the silk-material insoluble.

Exemplary excipients include, but are not limited to, a sugar or a sugar alcohol (e.g., sucrose, trehalose, sorbitol, mannitol, or a combination thereof), a divalent cation (e.g., Ca²⁺, Mg²⁺, Mn²⁺, and Cu²⁺), and/or buffers. In some embodiments, the concentration of an excipient can be used to modify the porosity of the matrix, e.g., with sucrose being used as the most common excipient for this purpose. Excipients may also be added to favor silk self-assembly into order beta-sheet secondary structure, and such excipients generally can participate in hydrogen bonding or charge interactions with silk to achieve this effect. Non-limiting examples of excipients that can be used to favor silk self-assembly into order beta-sheet secondary structure include monosodium glutamate (e.g., L-glutamic acid), lysine, sugar alcohols (e.g., sorbitol and/or glycerol), and solvents (e.g., DMSO, methanol, and/or ethanol).

In some embodiments, the sugar or the sugar alcohol is sucrose present in an amount less than 70% (w/v), less than 60% (w/v), less than 50% (w/v), less than 40% (w/v), less than 30% (w/v), less than 20% (w/v), less than 10% (w/v), less than 9% (w/v), less than 8% (w/v), less than 7% (w/v), less than 6% (w/v), or 5% (w/v) or less, e.g., immediately before drying.

In some embodiments, the sugar or the sugar alcohol is sucrose present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., immediately before drying.

In some embodiments, the sugar or the sugar alcohol is trehalose present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., immediately before drying.

In some embodiments, the sugar or the sugar alcohol is sorbitol present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., immediately before drying.

In some embodiments, the sugar or the sugar alcohol is glycerol present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., immediately before drying.

In some embodiments, the vaccine preparation further comprising a divalent cation. In some embodiments, the divalent cation is selected from the group consisting of Ca²⁺, Mg²⁺, Mn²⁺, and Cu²⁺. In some embodiments, the divalent cation is present in the preparation, e.g., immediately before drying, in an amount between 0.1 mM and 100 mM. In some embodiments, the divalent cation is present in the preparation, e.g., immediately before drying, in an amount between 10⁻⁷ and 10⁻⁴ moles per standard dose of viral immunogen. In some embodiments, the divalent cation is present in the preparation immediately before drying in an amount between 10⁻¹⁰ to 2×10⁻³ moles.

In some embodiments, the vaccine preparation further comprises poly(lactic-co-glycolic acid) (PGLA).

In some embodiments, the viral vaccine preparation further comprising a buffer, e.g., immediately before drying. In some embodiments, the buffer has buffering capacity between pH 3 and pH 8, between pH 4 and pH 7.5, or between pH 5 and pH 7. In some embodiments, the buffer is selected from the group consisting of HEPES and a CP buffer. In some embodiments, the buffer is present in the preparation, e.g., immediately before drying, in an amount between 0.1 mM and 100 mM. In some embodiments, the buffer is present in an amount between 10⁻⁷ and 10⁻⁴ moles per standard dose of viral immunogen. In some embodiments, the buffer is present in an amount between 10⁻¹⁰ to 2×10⁻³ moles.

In addition, the vaccine can also be formulated as a depot, gel, or hydrogel preparation. Such long acting formulations can be administered by implantation (for example subcutaneously or intramuscularly) or by intramuscular injection. Thus, for example, the vaccine can be formulated with suitable polymeric or hydrophobic materials (for example as an emulsion in an acceptable oil) or ion exchange resins, or as sparingly soluble derivatives, for example, as a sparingly soluble salt.

In one embodiment, the vaccine is administered via an implantable infusion device, e.g., a pump (e.g., a subcutaneous pump), an implant or a depot. Implantable infusion devices typically include a housing containing a liquid reservoir which can be filled transcutaneously by a hypodermic needle penetrating a fill port septum. The medication reservoir is generally coupled via an internal flow path to a device outlet port for delivering the liquid through a catheter to a patient body site. Typical infusion devices also include a controller and a fluid transfer mechanism, such as a pump or a valve, for moving the liquid from the reservoir through the internal flow path to the device's outlet port.

In some embodiments, the vaccine can be packages and/or formulated as a particle, e.g., a microparticle and/or a nanoparticle. Typically nanoparticles are from 10, 15, 20, 25, 30, 35, 45, 50, 75, 100, 150 or 200 nm or 200-1,000, e.g., 10, 15, 20, 25, 30, 35, 45, 50, 75, 100, 150, or 200, or 20 or 30 or 50-400 nm in diameter. Smaller particles tend to be cleared more rapidly form the system. Therapeutic agents, including vaccines, can be entrapped within or coupled, e.g., covalent coupled, or otherwise adhered, to nanoparticles.

Lipid- or oil-based nanoparticles, such as liposomes and solid lipid nanoparticles and can be used to can be used to deliver therapeutic agents, e.g., vaccines, described herein. Solid lipid nanoparticles for the delivery of therapeutic agents are descripbed in Serpe et al. (2004) Eur. J. Pharm. Bioparm. 58:673-680 and Lu et al. (20060 Eur. J. Pharm. Sci. 28: 86-95. Polymer-based nanoparticles, e.g., PLGA-based nanoparticles can be used to deliver agents described herein. These tend to rely on biodegradable backbone with the therapeutic agent intercalated (with or without covalent linkage to the polymer) in a matrix of polymer. PLGA is a widely used in polymeric nanoparticles, see Hu et al. (2009) J. Control. Release 134:55-61; Cheng et al. (2007) Biomaterials 28:869-876, and Chan et al. (2009) Biomaterials 30:1627-1634. PEGylated PLGA-based nanoparticles can also be used to deliver theraputic agents, see, e.g., Danhhier et al., (2009) J. Control. Release 133:11-17, Gryparis et al (2007) Eur. J. Pharm. Biopharm. 67:1-8. Metal-based, e.g., gold-based nanoparticles can also be used to deliver therapeutic agents. Protein-based, e.g., albumin-based nanoparticles can be used to deliver agents described herein. In some embodiments, a therapeutic agent can be bound to nanoparticles of human albumin.

A broad range of nanoparticles are known in the art. Exemplary approaches include those described in WO2010/005726, WO2010/005723 WO2010/005721, WO2010/121949, WO2010/0075072, WO2010/068866, WO2010/005740, WO2006/014626, 7,820,788, 7,780,984, the contents of which are incorporated herein in reference by their entirety.

### Dosages

Any dosage amount (e.g., a standard dose and/or a fractional dose) of a vaccine, antigen, and/or immunogen that is capable of eliciting an immune response (e.g., immunogenicity and/or broad-spectrum immunity) in a subject, e.g., when administered by a microneedle of the invention, may be used according to the methods described herein. In some embodiments, dose, e.g., the standard dose (e.g., human dose) for a vaccine, an antigen, and/or an immunogen (e.g., an influenza vaccine) is between about 0.1 μg and about 65 μg (e.g., between about 0.1 μg and about 10 μg, between about 0.1 μg and about 1 μg, between about 0.5 μg and about 5 μg, between about 5 μg and about 10 μg, between about 10 μg and about 20 μg, between about 20 μg and about 30 μg, between about 30 μg and about 40 μg, about 40 μg and about 50 μg, about 50 μg and about 65 μg, e.g., about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 μg). In some embodiments, the dose, e.g., standard human dose, for a vaccine described herein (e.g., an influenza vaccine) is approximately between about 1 μg and about 30 μg per strain, e.g., between about 5 μg and about 30 μg per strain (e.g., about 5, 6, 7, 8, 9, 10, 11, 12, 13, 14, 15, 16, 17, 18, 19, 20, 21, 22, 23, 24, 25, 26, 27, 28, 29, or 30 μg per strain). In some embodiments, the dose, e.g., fractional dose, for a vaccine described herein (e.g., an influenza vaccine) is no more than 1/X, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, or 100 or more, of the total dose (e.g., a standard dose). It is known in the art, that there is clinical precedent for dose-sparing when delivering influenza vaccine to the intradermal space (e.g., Fluzone ID), and this this dose is about 9 μg per strain. Accordingly, in some embodiments the total dosage amount of an influenza vaccine (e.g., Fluzone ID) that can be delivered by a microneedle of the invention can be between about 5 μg and 13 μg (e.g., about 5 μg, about 6 μg, about 7 μg, about 8 μg, about 9 μg, about 10 μg, about 11 μg, about 12 μg, or about 13 m).

Without wishing to be bound by theory, the total dosage amount (e.g., a standard dose) of a vaccine, antigen, and/or immunogen to be administered by a microneedle described herein can be divided between a plurality of microneedles (e.g., within a patch), such that a microneedle tip can comprises less than about 1% of the total dosage amount (e.g., in an array comprising about 121 microneedles), or at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% or more of the total dosage amount. In some embodiments, an implantable microneedle tip, as described herein, can comprise about 0.1 μg to about 65 μg (e.g., about 0.1 μg, about 0.2 μg, about 0.3 μg, about 0.4 μg, about 0.5 μg, about 0.6 μg, about 0.7 μg, about 0.8 μg, about 0.9 μg, about 1 μg, about 1 μg to about 10 μg, about 10 μg to about 20 μg, about 20 μg to about 30 μg, about 30 μg to about 40 μg, about 40 μg to about 50 μg, about 50 μg to about 65 μg) of a vaccine, antigen, and/or immunogen, as described herein.

In some embodiments, the vaccine dosage amount loaded into a microneedle patch can be manipulated via the concentration of antigen in the formulated solution that forms the needle tips, the volume of solution dispensed into each needle tip, and the total number of needles (the former two are more convenient means of varying dose). The dosage released into the skin is related to deployment efficiency (the portion of needle tips that are left behind in the skin after the patch is removed), and also the release profile over time and the residence time of the tips within the skin. Because of the continuous sloughing of skin from the epidermis, deeper deployment within the skin is related to longer residence time. Therefore, it is desirable to maximize the penetration depth of the needle tip (up to a limit defined by the depth of pain receptors within the skin, e.g., at a depth of between about 100 μm and about 600 μm), and also to have the antigen spatially concentrated toward the tip of the needle.

The formulations, compositions, articles, devices, and/or preparations described herein, including the implantable sustained-release tip formulation, are designed to not only sustain release of vaccine antigen over the duration, e.g., of tip retention in the dermis, but to also maintain stability of antigen during this period of time (e.g., at least about 1-2 weeks). In some embodiments, approximately 95-100% of the total dosage amount incorporated, e.g., in a formulation, composition, article, device, preparation, and/or microneedle described herein, can be expected to be available for delivery, e.g., into a subject, e.g., into a tissue of a subject, such as the skin, a mucous membrane, an organ tissue, a buccal cavity, a tissue, or a cell membrane. Without being bound by theory, successful deployment of a microneedle into the skin is at least about 50% and can be as high as 100% of an array (e.g., upon application at least about 50%, 60%, 70%, 80%, 90% or more (e.g., 100%) of the total number of microneedle comprising an array are successfully deployed within, e.g., the skin, for controlled- or sustained-release of a vaccine antigen). In some embodiments, a portion of antigen may not be released from the silk tips during the duration of deployment.

### Uses

The invention also provides methods for delivering a vaccine, an antigen, and/or an immunogen (e.g., an influenza vaccine) across a biological barrier (e.g., the skin). Such methods can include providing a formulation, composition, article, device, preparation, and/or microneedle described herein. For example, such methods can include providing at least one microneedle or at least one microneedle device described herein, wherein the microneedle or the microneedle device comprises a silk fibroin-based implantable tip having at least one vaccine, antigen, and/or an immunogen (e.g., an influenza vaccine); causing the microneedle or microneedle device to penetrate into the biological barrier (e.g., the skin); and allowing the vaccine, antigen, and/or an immunogen to be released from the implantable tips over a period of at least about 4 days (e.g., about 1, 2, 3, 4, 5, 6, 7, 8, 9, 10, 11, 12, 13, or 14 or more days, e.g., between about 4 days and about 14 days, e.g., between about 1-2 weeks, about 1-3 weeks, or about 1-4 weeks). In some embodiments, the vaccine, antigen, and/or an immunogen is released into the biological barrier through the degradation and/or dissolution of the implantable microneedle tips. In some embodiments, the microneedle or microneedle device is configured to administer the vaccine, antigen, and/or an immunogen in an amount and/or a duration that results in broad-spectrum immunity in the subject, e.g., an immunity against one or more viral antigens not present in the implantable sustained-release tip, e.g., an immunity against a drifted strain not present in the implantable sustained-release tip.

The invention also provides a method for providing broad-spectrum immunity to a virus, e.g., an influenza virus, in a subject, said method comprising administering a vaccine (e.g., a influenza vaccine) in an amount (e.g., a dosage) and/or over a time period sufficient to result in broad-spectrum immunity to a virus, e.g., results in an immune response (e.g., a cellular immune response and/or a humoral immune response) to a drifted strain of the virus, in the subject. In some embodiments, the vaccine is administered in a composition for the controlled- or sustained-release of the vaccine (e.g., for the controlled- or sustained-release of one or more viral antigens as described herein). In some embodiments, the vaccine is administered by a device for the controlled- or sustained-release of the vaccine (e.g., for the controlled- or sustained-release of one or more viral antigens as described herein). The vaccine can be administered into a subject, e.g., in to a tissue or cavity of the subject chosen from skin, mucosa, organ tissue, muscle tissue or buccal cavity.

In some embodiments, the methods described herein comprise administering a in an amount (e.g., a dosage) and/or over a time period sufficient to result in one or more of: (i) exposure in the subject to one or more antigens in the vaccine in an amount and/or period of time to result in broad spectrum immunity, e.g., to result in an immune response (e.g., a cellular immune response and/or a humoral immune response) to a drifted strain of the virus, in the subject; or (ii) a level of one or more antigens in the subject that is substantially steady, e.g., about 20%, 15%, 10%, 5%, or 1% to an amount, e.g., minimum amount, needed to result in an immune response (e.g., a cellular immune response and/or a humoral immune response) to the one or more antigens. In some embodiments, the composition or device for the controlled- or sustained-release of the vaccine is chosen from: a microneedle (e.g., a microneedle device, e.g., a microneedle patch, e.g., as described herein), an implantable device (e.g., a pump, e.g., a subcutaneous pump), an injectable formulation, a depot, a gel (e.g., a hydrogel), an implant, or a particle (e.g., a microparticle and/or a nanoparticle).

In some embodiments, the vaccine is administered, e.g., released by the composition or device for the controlled- or sustained-release of the vaccine, e.g., into the subject, in order to maintain a vaccine dosage (e.g., an antigen concentration) for a period of time sufficient to result in broad spectrum immunity, e.g., to result in an immune response (e.g., a cellular immune response and/or a humoral immune response) to a drifted strain of the virus, in the subject (e.g., wherein the period of time is about 1 to 21 days, e.g., about 5 to 10 days or about 5 to 7 days, e.g., about 1, 2, 3, 4, 5, 6, 7, 8, 9, 10, 11, 12, 13, 14, 15, 16, 17, 18, 19, 20, or 21 days). The composition or device for the controlled- or sustained-release of the vaccine can maintain antigen release and/or level in the subject over a sustained period of time. In some embodiments the composition or device for the controlled- or sustained-release of the vaccine maintains a continuous or non-continuous antigen release into the subject over a sustained period of time. The vaccine can administered, e.g., released by the composition or device for the controlled- or sustained-release, over a period of time comprising at least about one week, e.g., about 1-2 weeks, about 1-3 weeks, or about 1-4 weeks. In some embodiments, the vaccine is administered, e.g., released by the composition or device for the controlled- or sustained-release, over a period of time comprising at least about 4 days (e.g., about 4, 5, 6, 7, 8, 9, 10, 11, 12, 13, 14, 15, 16, 17, 18, 19, or 20 days, or more, e.g., between about 4 days and about 2 weeks, between about 4 days and about 1 week).

The vaccine can be administered in a dosage comprising between about 0.1 μg and about 65 μg per strain, e.g., 0.2 μg and about 50 μg per strain (e.g., about each of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 μg per strain). In some embodiments, at least about 1% of the dosage of the vaccine (e.g., at least about 0.5% to about 10%, at least about 5% to about 15% at least about 10% to about 20% of the dosage), e.g., released by the composition or device for the controlled- or sustained-release of the vaccine, e.g., into the subject, is maintained over a period of time comprising at least about 4 days (e.g., about 4, 5, 6, 7, 8, 9, 10, 11, 12, 13, or 14 days, or more, e.g., between about 4 days and about 2 weeks, between about 4 days and about 1 week).

In some embodiments, the vaccine is administered, e.g., released by the composition or device for the controlled- or sustained-release, in a plurality of fractional doses of a total dose (e.g., a standard dose) over a time period, e.g., such that an immune response and/or broad-spectrum immunity is achieved, wherein the amount of the vaccine administered in each of the fractional doses is no more than 1/X, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, or 100 or more, of the total dose (e.g., a standard dose) of the vaccine.

In some embodiments, the vaccine is administered, e.g., released by the composition or device for the controlled- or sustained-release of the vaccine, e.g., into the skin of the subject, in a plurality of doses equivalent to a percentage of a total dose (e.g., a percentage of a standard dose) over a time period, e.g., such that broad-spectrum immunity is achieved, wherein the amount of the vaccine administered in each of the plurality of doses is about X %, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, 100, 125, 150, 175, 200, 300, 400, or 500 or more, of the total dose (e.g., a standard dose) of the vaccine.

The vaccine can be administered according to any of the methods described herein such that broad-spectrum immunity is achieved, e.g., such that an immune response, e.g., a cellular immune and/or humoral immune response to a drifted strain is achieved.

Without wishing to be bound by theory, a subject exposed to and/or infected with a first influenza virus can develop an immune response (e.g., a cellular immune and/or humoral immune response) resulting in the creation of an antibody against that first influenza virus. As antigenic changes (e.g., mutations) accumulate in the first influenza virus over time, the subject's antibodies created against the first influenza virus may no longer recognize the drifted virus (e.g., the antigenically different strain). Using the methods, dosage regimens, microneedles, and microneedle devices described herein, broad-spectrum immunity can be conferred to a subject exposed to, infected with, and/or at risk of infection with an influenza virus. Further, using the methods, dosage regimens, microneedles, and microneedle devices described herein, improved immunogenicity and/or broad-spectrum immunity can be conferred to a subject, e.g., as compared to traditional burst release administration of vaccine. For example, improved immunogenicity and/or broad-spectrum immunity detectable in a subject can be greater (e.g., 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold or more greater) as compared to traditional burst release administration of vaccine, e.g., the administration of a single-dose or a bolus administration of the vaccine.

In some embodiments, the implantable sustained-release tip or the vaccine comprises a first influenza strain and administration of a dose of the first influenza strain (e.g., a first influenza A, B, C, and/or D strain as described herein) to the subject results in the development of broad-spectrum immunity to a second influenza strain (e.g., a drifted influenza A, B, C, and/or D strain as described herein) not present in the implantable sustained-release tip or the vaccine.

In some embodiments, the subject (e.g., the human subject) is a pediatric subject, an adult subject, or an elderly subject. The subject may have been exposed to, infected with, and/or at risk of infection with an influenza virus (e.g., a particular strain of an influenza virus). Such a risk may be due to the health status or age of the subject and/or travel to a region where a particular strain of influenza virus is prevalent.

In some embodiments, the invention provides methods of providing a controlled- or sustained-release of a vaccine in a subject. The controlled- or sustained-release of the vaccine can achieve an improved immunogenicity and/or broad-spectrum immunity, as compared to traditional burst release administration of vaccine. Without wishing to be bound by theory, an method of administering a vaccine described herein and/or a controlled- or sustained-release rate, e.g., by a composition and/or a microneedle described herein, that mimics the natural exposure pattern of a subject (e.g., a human subject) to a virus can provide enhanced immunity and/or broad-spectrum immunity to a subject, as compared to traditional single-dose vaccine administration modalities.

In some embodiments, a desired amount of at least one vaccine, antigen, and/or immunogen (e.g., an influenza vaccine) can be released from the microneedle (e.g., implantable mironeedle tip) described herein in a sustained manner over a pre-defined period of time. In some embodiments, at least about 5% of a vaccine, an antigen, and/or an immunogen (e.g., an influenza vaccine), e.g., at least about 10%, at least about 15%, at least about 20%, at least about 30%, at least about 40%, at least about 50%, at least about 60%, at least about 70%, at least about 80%, at least about 90%, at least about 95%, or at least about 97%, about 98%, or about 99%, or 100% of the vaccine, antigen, and/or an immunogen (e.g., an influenza vaccine), can be released from the microneedle (e.g., implantable microneedle tips) over a pre-defined period of time. In such embodiments, the desired amount (e.g., a dose, such as a standard dose of a vaccine) of the vaccine, antigen, and/or immunogen (e.g., an influenza vaccine) can be released from the microneedle over seconds, minutes, hours, months and/or years. In some embodiments, the desired amount (e.g., a dose, such as a standard dose of a vaccine) of the vaccine, antigen, and/or immunogen (e.g., an influenza vaccine) can be released from the microneedle upon insertion into a biological barrier, e.g., within 5 seconds, within 10 seconds, within 30 seconds, within 1 minute, within 2 minutes, within 3 minutes, within 4 minutes, within 5 minutes or longer. In some embodiments, the desired amount (e.g., a dose, such as a standard dose of a vaccine) of the vaccine, antigen, and/or immunogen (e.g., an influenza vaccine) can be released from the microneedle over a period of at least about 1 hour, at least about 2 hours, at least about 3 hours, at least about 6 hours, at least about 12 hours, at least about 1 day, at least about 2 days, at least about 3 days, at least about 4 days, at least about 5 days, at least about 6 days, at least about 1 week, at least about 2 weeks, at least about 1 month, at least about 2 months, at least about 3 months, at least about 6 months or longer. In some embodiments, the desired amount (e.g., a dose, such as a standard dose of a vaccine) of the vaccine, antigen, and/or immunogen (e.g., an influenza vaccine) can be released from the microneedle over about 1 year or longer.

In some embodiments, the invention provides methods for enhancing an immune response to a virus in a subject. In some embodiments, the presence of a cell-mediated immunological response can be determined by any art-recognized methods, e.g., proliferation assays (CD4+ T cells), CTL (cytotoxic T lymphocyte) assays (see Burke, supra; Tigges, supra), or immunohistochemistry with tissue section of a subject to determine the presence of activated cells such as monocytes and macrophages after the administration of an immunogen. One of skill in the art can readily determine the presence of humoral-mediated immunological response in a subject by any well-established methods. For example, the level of antibodies produced in a biological sample such as blood can be measured by western blot, ELISA or other methods known for antibody detection. In some embodiments, an elevated hemagglutination inhibition (HAI) antibody titer is detectable in the blood of the subject for the duration of a complete flu season post immunization.

In some embodiments, the immune response and/or the broad-spectrum immunity is a cellular immune and/or humoral immune response comprising: (i) an elevated hemagglutination inhibition (HAI) antibody titer detectable in the blood of the subject, e.g., detectable at least 3, 4, 5, 6, 7, 8, 9, 10, 11, 12, 13, 14, 15, 16, 17, 18, 19, 20, 21, 22, 23, 24, 25, 26, 27, 28, 29, and/or 30-weeks or more post immunization; (ii) an elevated anti-influenza IgG titer detectable in the blood of the subject, e.g., detectable at least 1, 2, 3, 4, 5, 6, 7, 8, 9, 10, 11 and/or 12-months or more post immunization; and/or (iii) a level of antibody secreting plasma cells (ASC) against the virus, e.g., the influenza virus, detectable in the bone marrow of the subject, e.g., detectable at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, and/or 34-weeks or more post immunization. In some embodiments, the elevated HAI antibody titer is to a drifted influenza A, B, C, and/or D strain. In some embodiments, the elevated anti-influenza IgG titer is to a drifted influenza A, B, C, and/or D strain. In some embodiments, the immune response is a cellular immune response comprising an increase in the level of IFNγ secreting cell in the blood of the subject, e.g., at least 2, 3, 4, 5, 6, 7, 8, 9, 10, 11, and/or 12-weeks or more post immunization, e.g., by a microneedle described herein.

In some embodiments, the elevated HAI antibody titer, the elevated anti-influenza IgG titer, the level of antibody secreting plasma cells (ASC) against the virus, and/or the level of IFNγ secreting cells detectable in the subject is greater (e.g., 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold or more greater) as compared to the administration of a single-dose or a bolus administration of the vaccine.

In some embodiments, broad-spectrum immunity can be characterized by measuring the percent seroconversion in a subject. For example, broad-spectrum immunity can comprise a percent seroconversion, e.g., based on the elevated HAI antibody titer detectable in the blood of the subject, e.g., at 6-month post immunization greater than about 20% (e.g., 20%, 25%, 30%, 35%, 40%, 45%, 50%, 55%, 60%, 65%, 70%, 75%, 80%, 85%, 90%, or 95% or more, e.g., 100%). Such a level of seroconversion associated with broad-spectrum immunity conferred by using the methods, dosage regimens, microneedles, and microneedle devices described herein can be greater (e.g., 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold or more greater) as compared to a level of seroconversion obtained by traditional burst release administration of vaccine, e.g., the administration of a single-dose or a bolus administration of the vaccine.

Combination Therapies

The microneedles and microneedle devices (e.g., microneedle patches) described herein may be manufactured by precision filling of each individual microneedle tip to enable different patterns of vaccine delivery, dosing schemes, and combination administration of a vaccine with an additional therapeutic agent. The methods of immunization, vaccine delivery, and dosing described herein may comprise combination administration of a vaccine with an additional therapeutic agent. In some embodiments, an additional therapeutic agent may be formulated in the same tip as a vaccine. In some embodiments, an additional therapeutic agent may be formulated with the vaccine. For example, adjuvants to boost immune response to co-delivered antigen could be delivered in the same microneedle tip and/or vaccine. Without wishing to be bound by theory, such a combination therapy could include adjuvants to drive stronger cellular immune responses and/or mucosal responses. Moreover, additional influenza antigens could be delivered for heterologous “prime/boost-like” immunization, e.g., primary immunization with an HA antigen from various influenza strains and a boost (e.g., provided via controlled- or sustained-release or distinct kinetic pattern from “prime”) with a different antigen (e.g., a drifted strain, a hemagglutinin stem, m2e protein, or NA).

Formulation compatibility may limit whether two given therapeutic agents can be co-formulated to be dispensed into the same needle tip. In case co-formulation is not possible, the manufacturing process can be adapted in order to dispense a first formulation into a portion of the needle array and then dispense a second formulation into a different portion of the needle array. Different formulations can also receive different process treatments after filling. For instance, if the first formulation will be for controlled- or sustained-release and the silk will be rendered less soluble via water annealing, while the second formulation will be for burst release with no annealing, the second formulation can be dispensed after the annealing step. The manufacturing approach is flexible so other process sequences are possible.

In some embodiments, the invention also provides methods for combination therapies, wherein a microneedle or microneedle device of the invention can be fabricated to administer at least one additional therapeutic agent. Various forms of a therapeutic agent can be used which are capable of being released from the microneedles described herein into adjacent tissues or fluids upon administration to a subject. In some embodiments, an additional therapeutic agent can be included within the base layer and/or within the implantable tip.

Examples of additional therapeutic agents that can be used according to the methods of the invention, e.g., incorporated into a microneedle of the invention, e.g., during fabrication, include steroids and esters of steroids (e.g., estrogen, progesterone, testosterone, androsterone, cholesterol, norethindrone, digoxigenin, cholic acid, deoxycholic acid, and chenodeoxycholic acid), boron-containing compounds (e.g., carborane), chemotherapeutic nucleotides, drugs (e.g., antibiotics, antivirals, antifungals), enediynes (e.g., calicheamicins, esperamicins, dynemicin, neocarzinostatin chromophore, and kedarcidin chromophore), heavy metal complexes (e.g., cisplatin), hormone antagonists (e.g., tamoxifen), non-specific (non-antibody) proteins (e.g., sugar oligomers), oligonucleotides (e.g., mRNA sequences or antisense oligonucleotides that bind to a target nucleic acid sequence), peptides, proteins, antibodies, photodynamic agents (e.g., rhodamine 123), radionuclides (e.g., 1-131, Re-186, Re-188, Y-90, Bi-212, At-211, Sr-89, Ho-166, Sm-153, Cu-67 and Cu-64), toxins (e.g., ricin), and transcription-based pharmaceuticals.

### Exemplary Kits

In certain embodiments, the invention relates to a package or kit comprising a microneedle described herein (e.g., a microneedle including a vaccine, antigen, and/or an immunogen as described herein, such as an influenza virus). In some embodiments, the invention relates to a package or kit comprising a vaccine described herein (e.g., a vaccine, antigen, and/or an immunogen as described herein, such as an influenza virus). In some embodiments, the kit can further comprise an additional therapeutic for combination therapy with the microneedle. In some embodiments, the kits can further comprise a disinfectant (e.g., an alcohol swab). In some embodiments, such packages, and kits described herein can be used for vaccination purposes, e.g., to achieve broad-spectrum immunity in a subject as described herein.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1. Sustained Intradermal Delivery of Influenza Vaccine Generates Improved Cellular Responses and Stronger, Longer-Lasting Antibody Responses

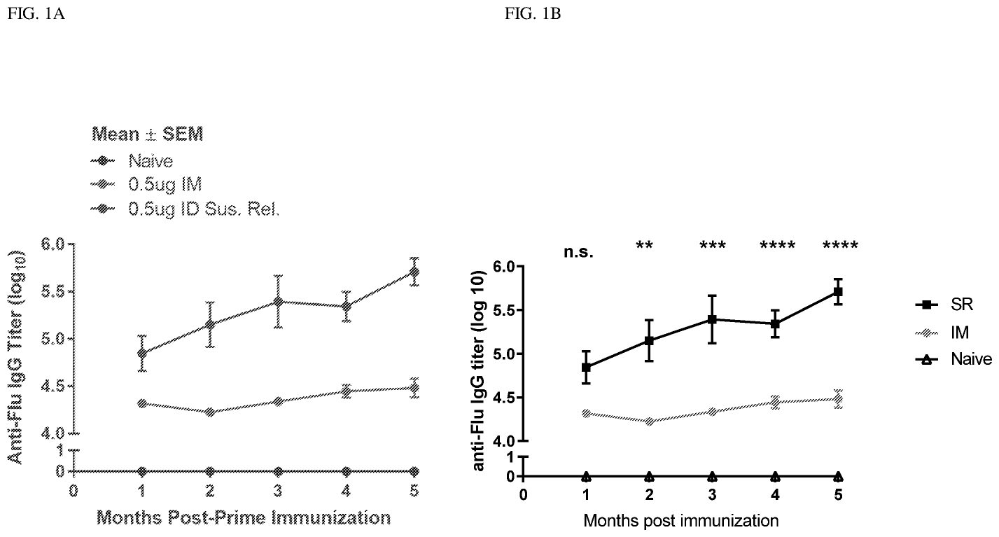

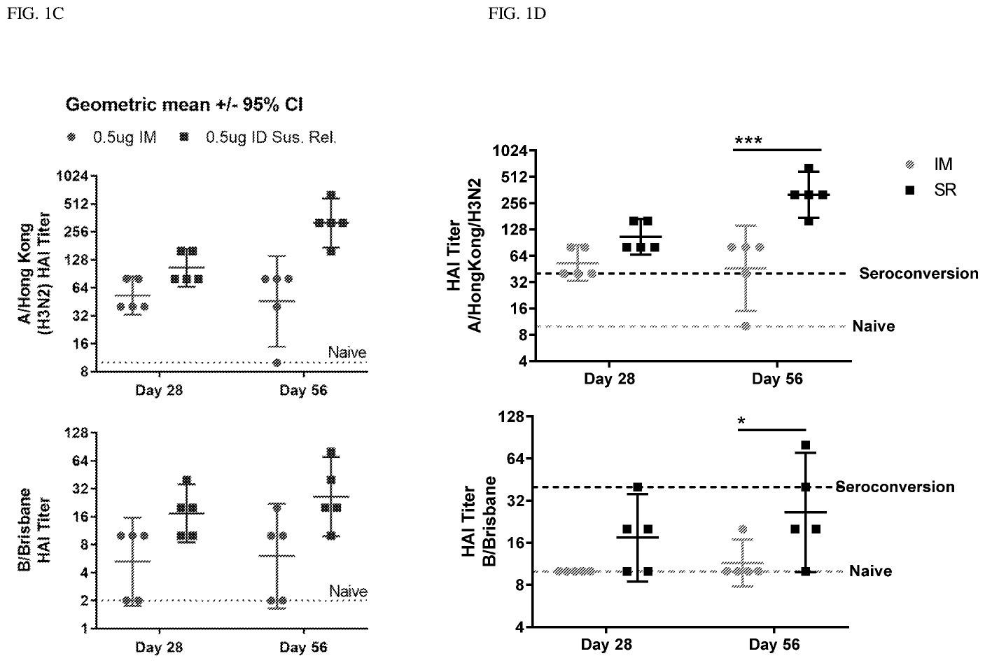

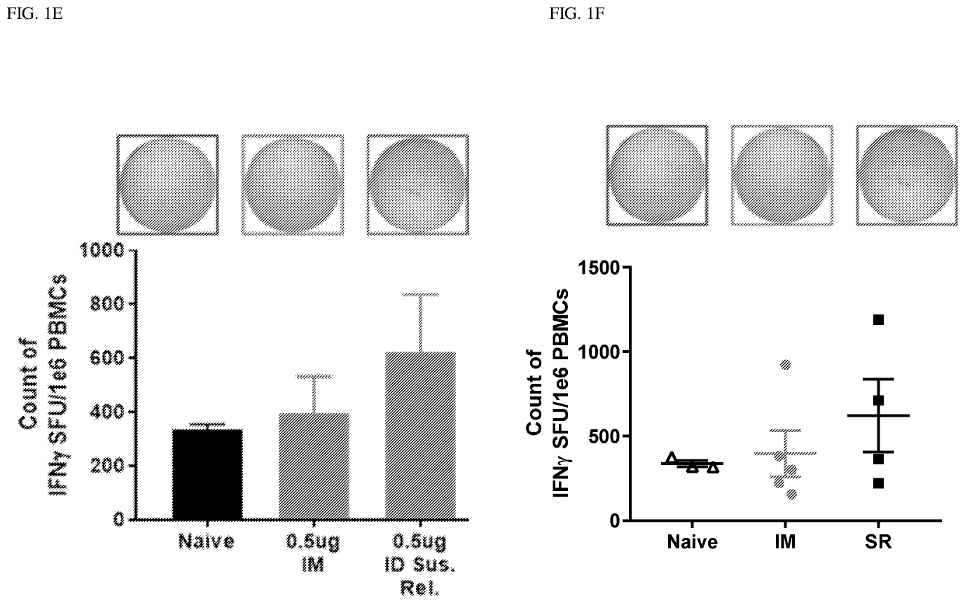

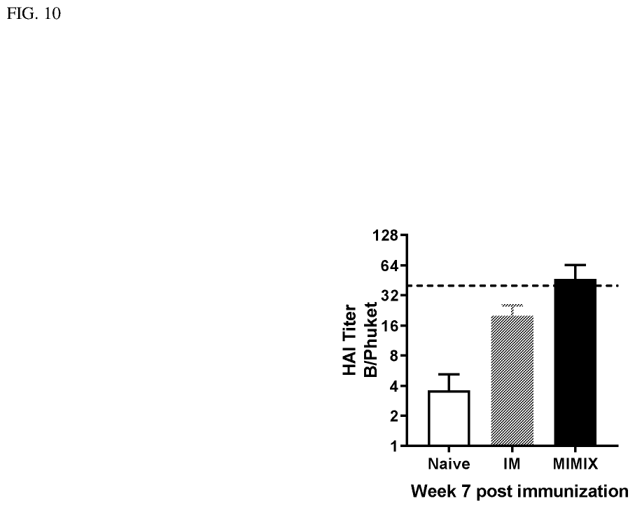

Balb/c mice were immunized with FluzoneHD at 0.5 ug/strain either by intramuscular injection or by intradermal injections of fractional doses for a total of 10 days. Anti-flu IgG responses were measured by ELISA. As shown in FIGS. 1A-1B, 10 day controlled- or sustained-release of vaccine results in significantly higher titers compared to equivalent intramuscular injections. Haemagglutination inhibition titers above 40 are known correlates of protection against infection. HAI titers for A/HongKong/H3N2 and B/Brisbane were measured at day 28 and 56 post immunization. At both time points, higher HAI titers were observed upon intradermal controlled- or sustained-release than IM injections (FIGS. 1C-1D) indicating higher correlates of protection by controlled- or sustained-release. T cell responses following vaccination were measured at week 12 by ELISPOT. A trend towards increased IFNγ+ cells in peripheral blood was observed upon sustained intradermal vaccine delivery when compared to IM injections (FIGS. 1E-1F). Taken together, these results indicate that sustained delivery of a vaccine against influenza results in stronger humoral and cellular responses than equivalent dose delivered by conventional intramuscular injections.

### Example 2. Controlled- or Sustained-Release Microneedle Formulation and Fabrication

Trivalent influenza vaccine (TIV) (Fluzone® High-Dose, 2017-18 formula, Sanofi-Pasteur, Swiftwater, Pa.) was prepared for microneedle device fabrication through processing to remove excess detergent and to concentrate HA antigen. 10 doses of TIV were run serially through detergent removal columns (Pierce™ Detergent Removal Spin Column, 2 mL, ThermoFisher 87778) to remove Triton X-100 (octyl phenol ethoxylate) detergent, a byproduct of manufacturing used to split influenza virus. An aliquot of material was collected and reserved for analysis via size exclusion chromatography (HPLC-SEC) to confirm absence of free detergent peaks. The remaining material was concentrated in 10 kDa spin filters (Amicon Ultra 0.5 mL, Fischer Sci 501096) through up to 3 10-minute spins at 15000 rpm. An aliquot of material was run on HPLC-SEC to determine concentration of flu antigens against initial vaccine. Comparison of area-under-the-curve (AUC) for pre-concentration and post-concentration material was used to determine the concentration of the processed antigen stock. 100 uL of stock was mixed with 85.6 uL of silk fibroin (60 MB) and 64.4 uL of Milli-Q water to generate a 5% (w/v) silk fibroin, 192 ug/mL HA (per strain) solution to be printed into microneedle molds.

Tip Filling: 20 nL of formulation was printed using vision-guided dispensing (Biodot AD3420) into a PDMS microneedle mold.

Tip Fill Inspection: Printing was visually assessed under stereomicroscope for defects, including misaligned prints, incompletely filled needle cavities, and foreign debris.

Tip Dry: Filled microneedle molds were dried under controlled 20% RH conditions overnight (14-20 hours).

Tip Anneal: Dried tips were water annealed at 37C for four hours, through placement of molds in a vacuum desiccator filled with Milli-Q water, applying vacuum for 5 minutes, then closing vacuum valve and moving desiccator to 37C incubator.

Tip Dry: after annealing tips were again dried under controlled 20% RH conditions overnight (14-20 hours).

Base Filling: A solution of 40% (w/v) hydrolyzed gelatin (Gelita) and 10% (w/v) sucrose (Sigma-Aldrich) was pipetted onto microneedle molds and filled via centrifugation at 3900 rpm for 2 minutes.

Base Fill Inspection: Base filling is also assessed visually by stereomicroscope for the appearance of needle cavities that were not entirely filled. Re-filling and re-centrifugation is performed if lack of fill is observed.

Base Drying: Base solution is dried under controlled 20% RH conditions overnight (14-20 hours).

Backing Apply: Whatman 903 cards were punched into 12 mm discs and applied to pre-wetted (10 uL Milli-Q water) dried gelatin base.

Backing Dry: Devices were dried under controlled 20% RH conditions for 2 hours before demolding.

Demolding: Devices were manually removed from microneedle molds by carefully bending the mold away from the device while holding device stationary.

Demold Inspect: Devices were inspected for complete demolding under stereomicroscope; incompletely demolded devices were discarded.

### Example 3. Immunization Via Controlled- or Sustained-Release Silk Microneedles Improves Humoral and Cellular Responses

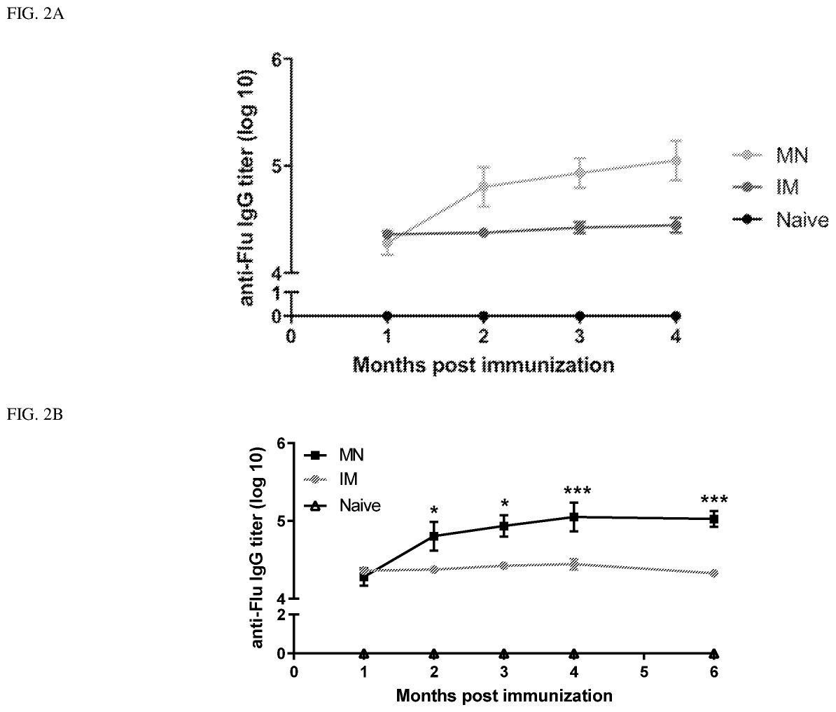

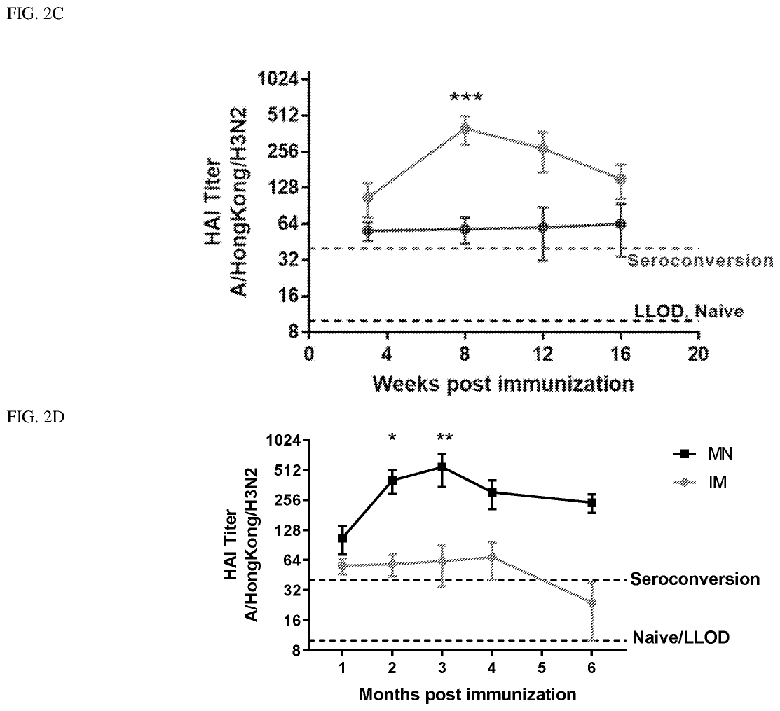

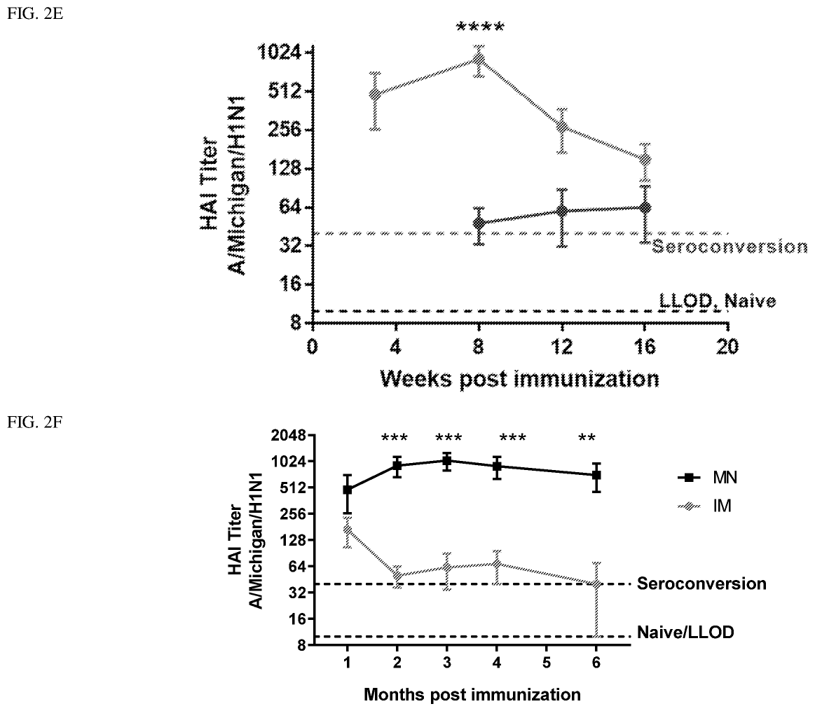

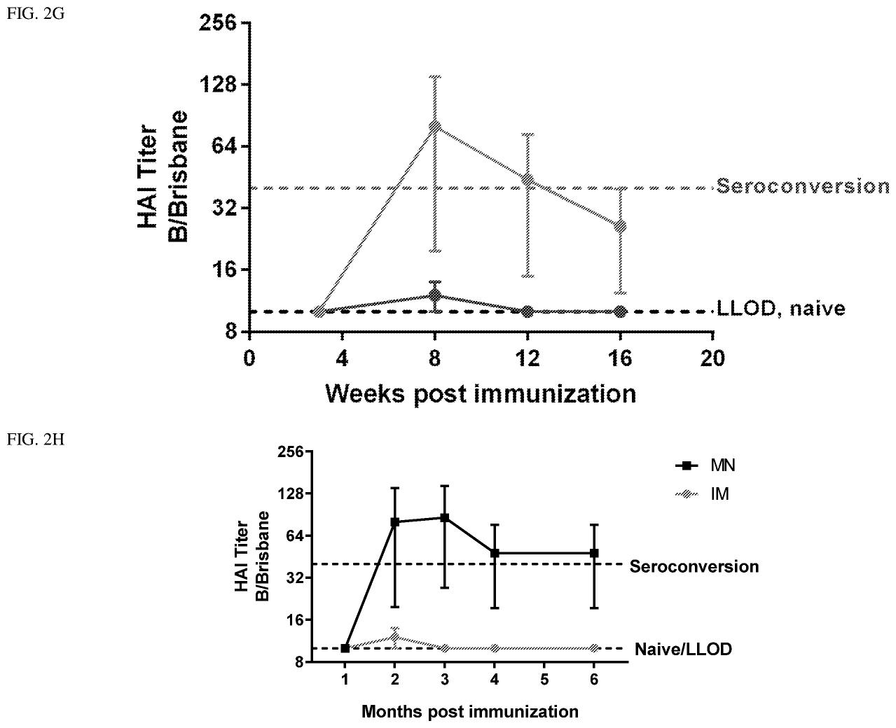

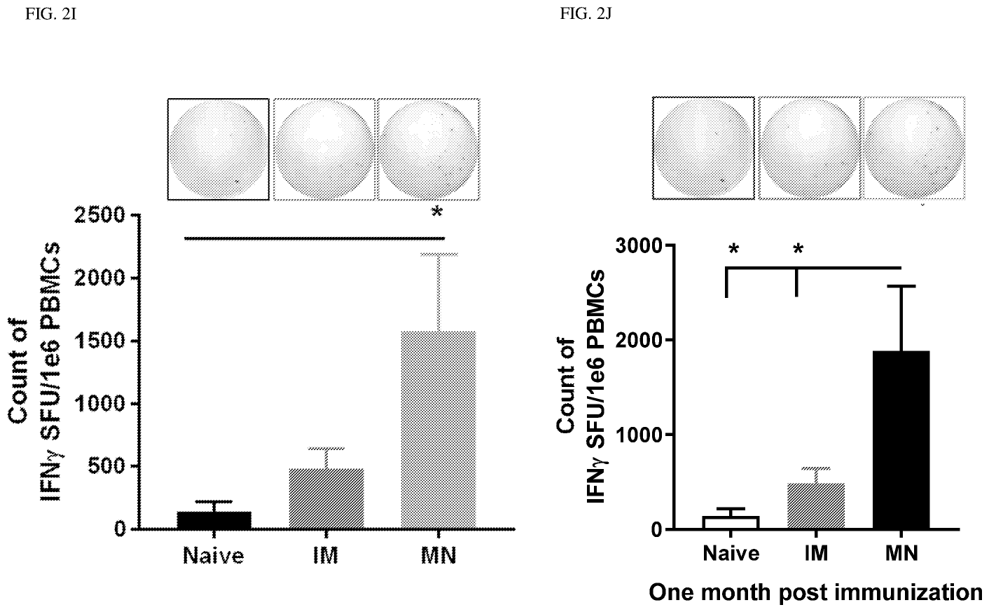

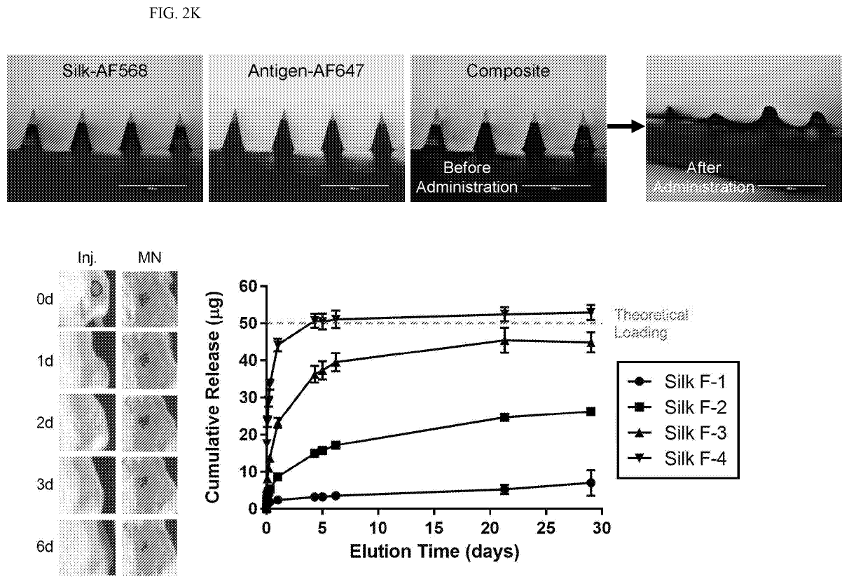

Balb/c mice were immunized by either intramuscular injections (IM) or microneedles that can sustain release the vaccine in the skin (MN). Following immunization, the anti-flu IgG titers were measured by ELISA. As shown in FIGS. 2A-2B, a 3-5 fold increase in titers is observed for 4 months and 6 months post immunization with MN compared to IM injection. HAI titers for the 3 strains, A/Hong Kong/H3N2, A/Michigan/H1N1 and B/Brisbane were measured at months 1, 2, 3, 4, and 6 post immunization (FIGS. 2C-2H). Significantly higher HAI titers were observed with MN with complete seroconversion maintained at month 4 compared to IM injection (FIGS. 2C and 2E). Significantly higher HAI titers were observed with MN with complete seroconversion maintained at month 6 compared to IM injection for the two A strains and a trend towards improved seroconversion for the B lineage (FIGS. 2D, 2F, and 2H). IFNγ cellular responses in peripheral blood was also significantly higher upon MN delivery of vaccine than IM delivery (FIG. 2E-2F). At week 4 post vaccination, significantly higher vaccine specific IFNγ+ cells in peripheral blood was determined by ELISPOT for MN delivery (FIG. 2I-2J). These results demonstrate the enhanced immunogenicity of vaccination possible though microneedle delivery.

### Example 4. Controlled- or Sustained-Release of Influenza Vaccine Results in an Immune Response, e.g., Humoral and Cellular Immune Responses, Against Drifted Strains of Influenza

### Controlled- or Sustained-Release of Influenza Vaccine Generates Higher HAI Titers Against Drifted H3N2 Strain of Influenza

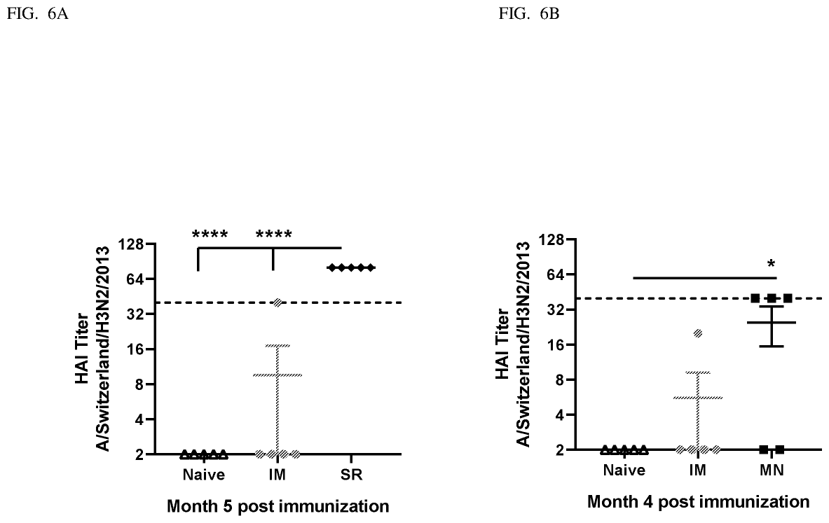

Balb/c mice were immunized with FluzoneHD at 0.5 ug/strain either by intramuscular injection or by intradermal injections of fractional doses for a total of 10 days or by application of the MIMIX (microneedle, MN) patch. HAI titers for A/Switzerland/H3N2/2013 (a strain that was not included in the vaccine) were measured at month 4 and 5 (days 120 and 150) post immunization respectively. As shown in FIGS. 6A-6B, 10 day controlled- or sustained-release of vaccine (SR) results in significantly higher titers to the drifted strain compared to equivalent intramuscular injections. Haemagglutination inhibition titers above 40 are known correlates of protection against infection. MIMIX (MN) delivery also showed a trend towards increased HAI titers with 3 out of 5 mice achieving a HAI titer of 40 compared to no animals in the IM immunized group at month 4 post immunization indicating higher correlates of protection by controlled- or sustained-release.

### Controlled- or Sustained-Release of Influenza Vaccine Generates Higher HAI Titers Against Drifted H1N1 Strain of Influenza

Balb/c mice were immunized with FluzoneHD at 0.5 ug/strain either by intramuscular injection or by intradermal injections of fractional doses for a total of 10 days or by application of the MIMIX patch. HAI titers for A/California/7/2009/H1N1 (a strain that was not included in the vaccine) were measured at month 6 (day 180) post immunization. As shown in FIG. 8, 10 day controlled- or sustained-release of vaccine results in significantly higher titers compared to equivalent intramuscular injections to the drifted vaccine strain. Haemagglutination inhibition titers above 40 are known correlates of protection against infection. MIMIX (MN) delivery also showed a trend towards increased HAI titers with 3 out of 5 mice achieving a HAI titer of 40 compared to no response in animals in the IM immunized group at month 4 post immunization indicating higher correlates of protection by controlled- or sustained-release.

### Controlled- or Sustained-Release of Influenza Vaccine Generates Higher HAI Titers Against B Lineage Strain not Included in the Vaccine

Balb/c mice were immunized with FluzoneHD at 0.5 ug/strain either by intramuscular injection or by intradermal injections of fractional doses for a total of 10 days or by application of the MIMIX patch. HAI titers for B/Phuket were measured at week 7 (day 49) post immunization. B/Phuket belongs to the Yamagata lineage that was not included in the vaccine. As shown in FIG. 10, sustained vaccine release from MIMIX showed a trend towards increase in HAI titers to this B lineage.

### Controlled- or Sustained-Release of Influenza Vaccine Generates More Long-Lived Plasma Cells in the Bone Marrow Against Both Vaccine Included and Drifted H3N2 Strains of Influenza

Balb/c mice were immunized with FluzoneHD at 0.5 ug/strain either by intramuscular injection or by intradermal injections of fractional doses for a total of 10 days. At month 8 (day 240) post immunization, animals were sacrificed and the cells from the bone marrow were isolated. A B cell ELISPOT was performed (following manufacturer's instructions, Immunospot) to measure antibody secreting plasma cells (ASC) against the vaccine included strain (A/Hong Kong/H3N2) and drifted strain (A/Switzerland/H3N2). As shown in FIGS. 7A-7B, fractional dosing of the vaccine over 10 days (SR) resulted in significantly higher number of both vaccine-specific and drifted strain specific ASCs.

### Sustained Release of Influenza Vaccine Generates More Long-Lived Plasma Cells in the Bone Marrow Against Both Vaccines Included and Drifted H1N1 Strains of Influenza

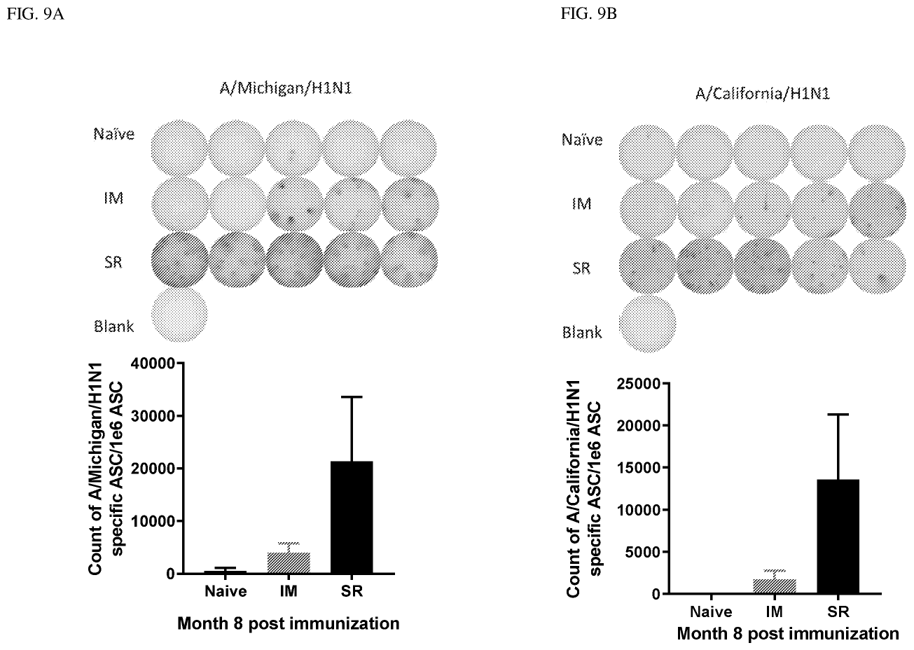

Balb/c mice were immunized with FluzoneHD at 0.5 ug/strain either by intramuscular injection or by intradermal injections of fractional doses for a total of 10 days. At month 8 (day 240) post immunization, animals were sacrificed and the cells from the bone marrow were isolated. A B cell ELISPOT was performed (following manufacturer's instructions, Immunospot) to measure antibody secreting plasma cells (ASC) against the vaccine included strain (A/Michigan/H1N1) and drifted strain (A/California/H1N1). As shown in FIGS. 9A-9B, fractional dosing of the vaccine over 10 days (SR) showed a trend towards increase in both vaccine-specific and drifted strain specific ASCs.

### Significantly Higher Number of Both ASC Against the Vaccine Included B/Lineage Strain (B/Brisbane) and to the B/Yamagata Lineage (B/Phuket)

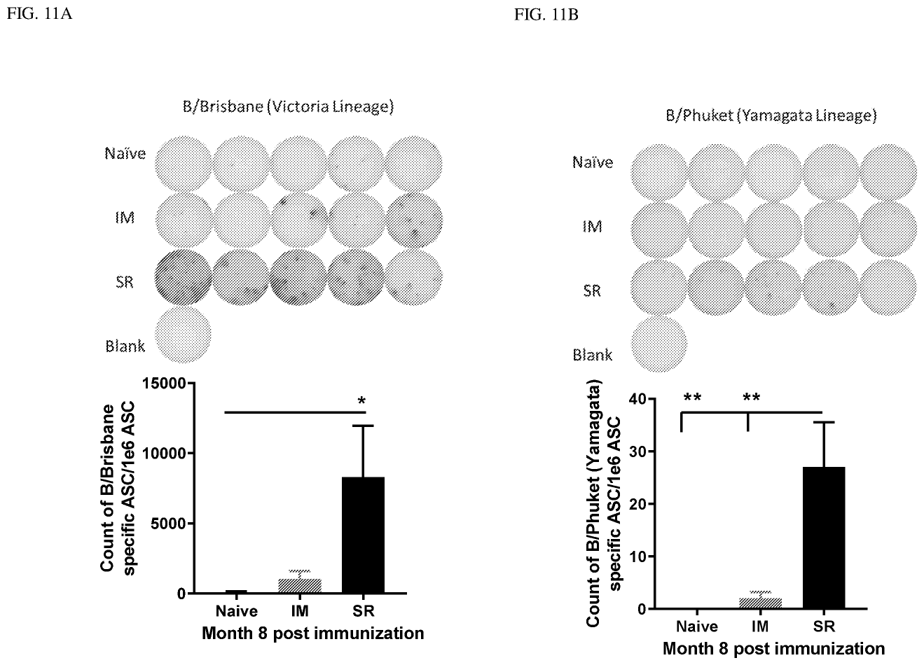

Balb/c mice were immunized with FluzoneHD at 0.5 ug/strain either by intramuscular injection or by intradermal injections of fractional doses for a total of 10 days. At month 8 (day 240) post immunization, animals were sacrificed and the cells from the bone marrow were isolated. A B cell ELISPOT was performed (following manufacturer's instructions, Immunospot) to measure antibody secreting plasma cells (ASC) against the vaccine included B/lineage strain (B/Brisbane) and to the B/Yamagata lineage (B/Phuket). As shown in FIGS. 11A-11B, fractional dosing of the vaccine over 10 days (SR) resulted in significantly higher number of both vaccine-specific and drifted strain specific ASCs.

### CONCLUSION

Controlled- or sustained-release leads to increased plasma cells and protective HAI titers against both vaccine and drifted influenza viruses, suggesting stronger and broader protection. Table 1, below, indicates the percent (%) seroconversion corresponding to the data in FIGS. 9, 11, and 13. Taken together, these results indicate that sustained delivery of a vaccine against influenza results in stronger HAI titers to drifted (non-vaccine) strains than equivalent dose delivered by conventional intramuscular injections. Table 2, below, indicates the fold increase post sustained vaccine release (SR) over intramuscular injection (IM) in the count of long-lived plasma cells in the bone marrow specific for vaccine included and drifted strains quantified in FIGS. 10, 12, and 14. Taken together, these results indicate that controlled- or sustained-release of the vaccine against influenza results in durable presence of antibody secreting plasma cells to drifted (non-vaccine) strains than equivalent dose delivered by conventional intramuscular injections.

TABLE 1
Percent (%) seroconversion based on HAI titers
to vaccine included and drifted strains

| | | | Sustained | |
|---|---|---|---|---|
| | | | Vaccine | Micronee- |
| % Seroconversion | | Intramuscular | Release | dle |
| per Strain | | Injection (IM) | (SR) | (MN) |
| H1N1 | A/Michigan (Vac) | 20 | 80 | 80 |
| | A/California (Drift) | 20 | 80 | 60 |
| H3N2 | A/Hong Kong (Vac) | 20 | 80 | 100 |
| | A/Switzerland* (Drift) | 0 | 80 | 60 |
| B | B/Brisbane (Vac) | 0 | — | 40 |
| | B/Phuket\hat{ } (Drift) | 20 | — | 66 |

*at month 4,
\hat{ }at week 7

TABLE 2
Fold increase post sustained vaccine release
(SR) over intramuscular injection (IM)

| Strain | | Fold increase, SR vs IM |
|---|---|---|
| H1N1 | A/Michigan (Vac) | 5.4 |
| | A/California (Drift) | 7.9 |
| H3N2 | A/Hong Kong (Vac) | 10.9 |
| | A/Switzerland (Drift) | 5.5 |
| B | B/Brisbane (Vac) | 7.9 |
| | B/Phuket (Drift) | 13.5 |

### EQUIVALENTS

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize, or be able to ascertain using no more than routine experimentation, many equivalents to the specific embodiments of the invention described specifically herein. Such equivalents are intended to be encompassed in the scope of the appended claims.

What is claimed is:

1. A microneedle comprising:
(i) a backing,
(ii) a dissolvable base comprising one, two, three, four or more of gelatin, polyethylene glycol (PEG), sucrose, carboxymethylcellulose (CMC), polyvinylpyrrolidone (PVP), polyvinyl alcohol (PVA), hyaluronate, maltose, and methyl cellulose applied to the backing,
(iii) an implantable sustained-release tip comprising a silk fibroin applied to the dissolvable base,
wherein the microneedle is configured to implant the sustained-release tip into the skin of a subject, e.g., a human subject, at a depth (e.g., a max penetration depth of the distal part of tip) of between about 100 μm and about 600 μm,
wherein the sustained-release tip comprises a silk fibroin, e.g., a regenerated silk fibroin and/or a recombinant silk fibroin,
wherein the sustained-release tip further comprises a vaccine (e.g., an influenza vaccine) in an amount sufficient to induce an immune response, e.g., a humoral immune response and/or a cellular immune response.

2. The microneedle of claim 1, wherein the dissolvable base comprising one of gelatin, polyethylene glycol (PEG), sucrose, carboxymethylcellulose (CMC), polyvinylpyrrolidone (PVP), polyvinyl alcohol (PVA), hyaluronate, maltose, and methyl cellulose.

3. The microneedle of claim 1, wherein the dissolvable base comprising two of gelatin, PEG, sucrose, CMC, PVP, PVA, hyaluronate, maltose, and methyl cellulose.

4. The microneedle of claim 1, wherein the dissolvable base comprising three of gelatin, PEG, sucrose, CMC, PVP, PVA, hyaluronate, maltose, and methyl cellulose.

5. The microneedle of claim 1, wherein the dissolvable base comprising four of gelatin, PEG, sucrose, CMC, PVP, PVA, hyaluronate, maltose, and methyl cellulose.

6. The microneedle of claim 1, wherein the dissolvable base comprising five of gelatin, PEG, sucrose, CMC, PVP, PVA, hyaluronate, maltose, and methyl cellulose.

7. The microneedle of claim 1, wherein the dissolvable base comprising six of gelatin, PEG, sucrose, CMC, PVP, PVA, hyaluronate, maltose, and methyl cellulose.

8. The microneedle of claim 1, wherein the dissolvable base comprising seven of gelatin, PEG, sucrose, CMC, PVP, PVA, hyaluronate, maltose, and methyl cellulose.

9. The microneedle of claim 1, wherein the dissolvable base comprising gelatin, PEG, sucrose, CMC, PVP, PVA, hyaluronate, maltose, and methyl cellulose.

10. The microneedle of any one of the preceding claims, wherein the dissolvable base comprising gelatin and sucrose.

11. The microneedle of any one of the preceding claims, wherein the dissolvable base comprises CMC.

12. The microneedle of any one of the preceding claims, wherein the dissolvable base comprises PVP.

13. The microneedle of any one of the preceding claims, wherein the dissolvable base comprises PVA.

14. The microneedle of any one of the preceding claims, wherein the dissolvable base comprises PVP and PVA.

15. The microneedle of any one of the preceding claims, wherein the dissolvable base comprises PVP, PVA, and sucrose.

16. The microneedle of any one of the preceding claims, wherein the dissolvable base does not comprise poly(acrylic acid) (PAA).

17. The microneedle of any one of the preceding claims, wherein the implantable sustained-release tip is configured to release a vaccine into the skin of the subject over a period of time comprising at least about 4 days (e.g., about 4, 5, 6, 7, 8, 9, 10, 11, 12, 13, or 14 or more days, e.g., between about 4 days and about 14 days, e.g., between about 1-2 weeks, about 1-3 weeks, or about 1-4 weeks).

18. The microneedle of any one of the preceding claims, wherein the implantable sustained-release tip is configured to release a vaccine into the skin of the subject over a period of time comprising about 1 week to about 2 weeks (e.g., about 7, 8, 9, 10, 11, 12, 13, or 14 days).

19. The microneedle of any one of the preceding claims, wherein immune response is a humoral immune response comprises:
(i) an elevated hemagglutination inhibition (HAI) antibody titer detectable in the blood of the subject, e.g., detectable at least 3, 4, 5, 6, 7, 8, 9, 10, 11, 12, 13, 14, 15, and/or 16-weeks post immunization; and/or
(ii) an elevated anti-influenza IgG titer detectable in the blood of the subject, e.g., detectable at least 1, 2, 3, 4, and/or 5-months post immunization.

20. The microneedle of any one of the preceding claims, wherein an elevated hemagglutination inhibition (HAI) antibody titer is detectable in the blood of the subject for the duration of a complete flu season post immunization.

21. The microneedle of any one of the preceding claims, wherein immune response is a cellular immune response comprising an increase in the level of IFNγ secreting cell in the blood of the subject, e.g., at least 2, 3, 4, 5, 6, 7, 8, 9, 10, 11, and/or 12-weeks post immunization.

22. The microneedle of any one of the preceding claims, wherein the dissolvable base comprises between about 10% and about 70% gelatin (e.g., hydrolyzed gelatin) (e.g., about 10%, about 20%, about 30%, about 40%, about 50%, about 60%, or about 70% gelatin).

23. The microneedle of any one of the preceding claims, wherein the dissolvable base comprises between about 1% and about 35% sucrose (e.g., about 1%, about 2%, about 3%, about 4%, about 5%, about 6%, about 7%, about 8%, about 9%, about 10%, about 15%, about 20%, about 25%, about 30%, or about 35% sucrose).

24. The microneedle of any one of the preceding claims, wherein the dissolvable base comprises between about 1% and about 35% CMC (e.g., about 1%, about 2%, about 3%, about 4%, about 5%, about 6%, about 7%, about 8%, about 9%, about 10%, about 15%, about 20%, about 25%, about 30%, or about 35% CMC).

25. The microneedle of any one of the preceding claims, wherein the dissolvable base comprises between about 10% and about 70% PVP (e.g., about 10%, about 20%, about 30%, about 40%, about 50%, about 60%, or about 70% PVP).

26. The microneedle of any one of the preceding claims, wherein the dissolvable base comprises between about 1% and about 35% PVA (e.g., e.g., about 1%, about 2%, about 3%, about 4%, about 5%, about 6%, about 7%, about 8%, about 9%, about 10%, about 15%, about 20%, about 25%, about 30%, or about 35% PVA).

27. The microneedle of any one of the preceding claims, wherein the dissolvable base comprises between about 1% and about 75% (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% hyaluronate).

28. The microneedle of any one of the preceding claims, wherein the dissolvable base comprises between about 1% and about 75% (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% maltose).

29. The microneedle of any one of the preceding embodiments, wherein the dissolvable comprises between about 1% and about 75% (e.g., about 1%, about 2%, about 3%, about 4%, about 5%, about 6%, about 7%, about 8%, about 9%, about 10%, about 15%, about 20%, about 25%, about 30%, or about 35%, about 40%, about 45%, about 50%, about 55%, about 60%, about 65%, about 70%, or about 75% methyl cellulose).

30. The microneedle of any one of the preceding claims, wherein the dissolvable base comprises about 40% hydrolyzed gelatin and about 10% sucrose w/v.

31. The microneedle of any one of the preceding claims, wherein the dissolvable base comprises up to about 50% w/v of PVP (e.g., PVP of 10 kD MW).

32. The microneedle of any one of the preceding claims, wherein the dissolvable base comprises up to about 20% PVA (e.g., 87% hydrolyzed PVA at 13 kD MW).

33. The microneedle of any one of the preceding claims, wherein the dissolvable base comprises CMC at up to about 10%.

34. The microneedle of any one of the preceding claims, wherein the dissolvable base comprises about 1% CMC (e.g., low-viscosity CMC).

35. The microneedle of any one of the preceding claims, wherein the dissolvable base comprises about 30% PVP and about 10% PVA.

36. The microneedle of any one of the preceding claims, wherein the dissolvable base comprises about 37% PVP, about 5% PVA, and about 15% sucrose.

37. The microneedle of any one of the preceding claims, wherein the implantable sustained-release tip comprises silk fibroin at about 1% w/v to about 10% w/v (e.g., about 1, 2, 3, 4, 5, 6, 7, 8, 9, or 10% w/v, or a silk fibroin having a molecular weight distribution according to FIG. 5, or, comprises silk fibroin in an amounta between about 20 μg to about 245 μg, e.g., per 121 microneedle array).

38. The microneedle of any one of the preceding claims, wherein the implantable sustained-release tip comprises a vaccine formulated in a 1% w/v to about 10% w/v (e.g., about 1, 2, 3, 4, 5, 6, 7, 8, 9, or 10% w/v) of 10 MB silk fibroin solution, or a silk fibroin solution according to FIG. 5.

39. The microneedle of any one of the preceding claims, wherein the implantable sustained-release tip comprises a vaccine formulated in a 1% w/v to about 10% w/v (e.g., about 1, 2, 3, 4, 5, 6, 7, 8, 9, or 10% w/v) of 60 MB silk fibroin solution, or a silk fibroin solution according to FIG. 5, e.g., a 100 kDa to 200 kDa (e.g., about 153 kDa) silk fibroin solution.

40. The microneedle of any one of the preceding claims, wherein the implantable sustained-release tip comprises a vaccine formulated in a 1% w/v to about 10% w/v (e.g., about 1, 2, 3, 4, 5, 6, 7, 8, 9, or 10% w/v) of 120 MB silk fibroin solution, or a silk fibroin solution according to FIG. 5, e.g., a 70 kDa to 150 kDa (e.g., about 100 kDa) silk fibroin solution.

41. The microneedle of any one of the preceding claims, wherein the implantable sustained-release tip comprises a vaccine formulated in a 1% w/v to about 10% w/v (e.g., about 1, 2, 3, 4, 5, 6, 7, 8, 9, or 10% w/v) of 180 MB silk fibroin solution, or a silk fibroin solution according to FIG. 5, e.g., a 36 kDa to 100 kDa (e.g., about 71 kDa) silk fibroin solution.

42. The microneedle of any one of the preceding claims, wherein the implantable sustained-release tip comprises a vaccine formulated in a 1% w/v to about 10% w/v (e.g., about 1, 2, 3, 4, 5, 6, 7, 8, 9, or 10% w/v) of 480 MB silk fibroin solution, or a silk fibroin solution according to FIG. 5, e.g., a 1 kDa to 60 kDa (e.g., about 16 kDa) silk fibroin solution.

43. The microneedle of claim 39, wherein the implantable sustained-release tip comprises a 5% wt/vol 60 MB silk fibroin solution.

44. The microneedle of any one of the preceding claims, wherein the implantable sustained-release tip comprises a standard dose of a vaccine.

45. The microneedle of any one of the preceding claims, wherein the standard dose of the vaccine (e.g., influenza vaccine) comprises between about 0.1 μg and about 65 μg per strain, e.g., 0.2 μg and about 50 μg per strain (e.g., about each of 0.1, 0.2, 0.3, 0.4, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 μg per strain).

46. The microneedle of claim 44 or 45, wherein the implantable sustained-release tip comprises at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% or more of the standard dose.

47. The microneedle of any one of claims 44-46, wherein the implantable sustained-release tip comprises about 0.1 μg to about 65 μg of vaccine (e.g., about 0.1 μg, about 0.2 μg, about 0.3 μg, about 0.4 μg, about 0.5 μg, about 0.6 μg, about 0.7 μg, about 0.8 μg, about 0.9 μg, about 1 μg, about 1 μg to about 10 μg, about 10 μg to about 20 μg, about 20 μg to about 30 μg, about 30 μg to about 40 μg, about 40 μg to about 50 μg, about 50 μg to about 65 μg of a vaccine).

48. The microneedle of any one of the preceding claims, wherein the length of the microneedle is between about 350 μm to about 1500 μm ((e.g., about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1050 μm, about 1100 μm, about 1150 μm, about 1200 μm, about 1250 μm, about 1300 μm, about 1350 μm, about 1400 μm, about 1450 μm, about 1500 μm).

49. The microneedle of any one of the preceding claims, wherein the height of the implantable sustained-release tip may extend to approximately half of the full height of the microneedle.

50. The microneedle of any one of the preceding claims, wherein the height of the implantable sustained-release tip is between about 75 μm to about 475 μm (e.g., about 75, about 100 μm, about 125 μm, about 150 μm, about 175 μm, about 200 μm, about 225 μm, about 250 μm, about 275 μm, about 300 μm, about 325 μm, about 375 μm, about 400 μm, about 425 μm, or about 475 μm).

51. The microneedle of any one of the preceding claims, wherein the implantable sustained-release tip comprises a tip radius between about 0.5 μm to about 25 μm (e.g., about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, or 25 μm).

52. The microneedle of any one of the preceding claims, wherein the implantable sustained-release tip comprises a tip radius between about 5 μm to about 10 μm (e.g., about 5, 6, 7, 8, 9, or 10 μm).

53. The microneedle of any one of the preceding claims, wherein the implantable sustained-release tip comprises an angle between about 5 degrees and about 45 degrees (e.g., about 5, 6, 7, 8, 9, 10, 15, 20, 25, 30, 35, 40, or 45 degrees).

54. The microneedle of any one of the preceding claims, wherein the backing is chosen from a solid support, e.g., a paper-based material, a plastic material, a polymeric material, or a polyester-based material (e.g., a Whatman 903 paper, a polymeric tape, a plastic tape, an adhesive-backed polyester tape, or other medical tape).

55. The microneedle of any one of the preceding claims, wherein the implantable sustained-release tip comprises an influenza vaccine.

56. The microneedle of claim 55, wherein the influenza vaccine comprises an influenza A vaccine, an influenza B vaccine, an influenza C vaccine, and/or an influenza D vaccine.

57. The microneedle of claim 55 or 56, wherein the influenza vaccine comprises an influenza A vaccine, optionally wherein the influenza A vaccine is a H1N1 vaccine and/or a H3N2 vaccine.

58. The microneedle of any one of claims 55-57, wherein the influenza vaccine comprises an influenza B vaccine, optionally wherein the influenza B vaccine is an B/Yamagata lineage and/or the B/Victoria lineage vaccine.

59. The microneedle of any one of claims 55-58, wherein the influenza vaccine comprises an influenza A vaccine (e.g., a H1N1 vaccine and/or a H3N2 vaccine) and an influenza B vaccine (e.g., an B/Yamagata lineage and/or the B/Victoria lineage vaccine).

60. A device, e.g., an array or patch, comprising a plurality of microneedles (e.g., two or more microneedles as described herein), e.g., a plurality of microneedles according to any one of claims 1-59.

61. The device of claim 60, wherein the microneedles of the plurality are the same, e.g., comprise the same implantable sustained-release tip, e.g., comprising the same therapeutic agent, e.g., the same immunogen, antigen or vaccine.

62. The device of claim 60, wherein two or more of the microneedles of the plurality are different, e.g., comprise two or more different implantable sustained-release tips, e.g., comprising two or more therapeutic agents, e.g., comprising a combination of two or more immunogens, antigens or vaccines, with or without one or more adjuvants.

63. The device of claim 60, which comprises at least 10%, 20%, 30%, 40%, 50%, 60%, 70%, 80%, 90% or more of a first implantable sustained-release tip relative to a further (e.g., second, third, fourth, fifth) implantable sustained-release tip.

64. The device of claim 60, wherein a total dosage amount (e.g., a standard dose) of a vaccine, antigen, and/or immunogen is divided between the plurality of microneedles (e.g., within a patch), such that the implantable sustained-release microneedle tip can comprises at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% or more of the total dosage amount.

65. The device of claims 60-63, wherein the implantable microneedle tip comprises about 0.1 μg to about 65 μg (e.g., about 0.1 μg, about 0.2 μg, about 0.3 μg, about 0.4 μg, about 0.5 μg, about 0.6 μg, about 0.7 μg, about 0.8 μg, about 0.9 μg, about 1 μg, about 1 μg to about 10 μg, about 10 μg to about 20 μg, about 20 μg to about 30 μg, about 30 μg to about 40 μg, about 40 μg to about 50 μg, about 50 μg to about 65 μg) of a vaccine, antigen, and/or immunogen, as described herein.

66. A method of providing immunity, e.g., broad-spectrum immunity, to a virus, e.g., an influenza virus, e.g., a drifted influenza A, B, C, and/or D strain, in a subject comprising contacting the skin of the subject with a microneedle of any one of claims 1-59.

67. A method of providing a sustained-release of a vaccine, e.g., an influenza vaccine, in a subject comprising contacting the skin of the subject with a microneedle of any one of claims 1-59.

68. A method of enhancing an immune response to a virus, e.g., an influenza virus, e.g., a drifted influenza A, B, C, and/or D strain, in a subject comprising contacting the skin of the subject with a microneedle of any one of claims 1-59.

69. The method of any one of claims 66-68, wherein the implantable sustained-release tip is configured to release a vaccine into the skin of the subject over a period of time comprising at least about one week, e.g., about 1-2 weeks, about 1-3 weeks, or about 1-4 weeks.

70. The method of claim 69, wherein the implantable sustained-release tip is configured to release a vaccine into the skin of the subject over a period of time comprising at least about 4 days (e.g., about 4, 5, 6, 7, 8, 9, 10, 11, 12, 13, or 14 days, or more, e.g., between about 4 days and about 2 weeks).

71. The method of any one of claims 66-58, wherein immune response is a humoral immune response comprises:
(i) an elevated hemagglutination inhibition (HAI) antibody titer detectable in the blood of the subject, e.g., detectable at least 3, 4, 5, 6, 7, 8, 9, 10, 11, 12, 13, 14, 15, 16, 17, 18, 19, 20, 21, 22, 23, 24, and/or 25-weeks post immunization;
(ii) an elevated anti-influenza IgG titer detectable in the blood of the subject, e.g., detectable at least 1, 2, 3, 4, 5 and/or 6-months post immunization, optionally wherein the elevated anti-influenza IgG titer is to a drifted influenza A, B, C, and/or D strain;

72. The method of claim 71, wherein an elevated hemagglutination inhibition (HAI) antibody titer is detectable in the blood of the subject for the duration of a complete flu season post immunization, optionally wherein the elevated HAI antibody titer is to a drifted influenza A, B, C, and/or D strain.

73. The method of claim 71 or 72, wherein immune response is a cellular immune response comprising an increase in the level of IFNγ secreting cell in the blood of the subject, e.g., at least 2, 3, 4, 5, 6, 7, 8, 9, 10, 11, and/or 12-weeks post immunization.

74. A method of producing a microneedle device, the method comprising:
providing a mold including a mold body with an array needle cavities having a predefined shape, e.g., a pyramid-shaped and/or of conical-shaped needle cavities, formed therein;
filling tips of the needle cavities with a composition consisting of a silk fibroin, antigen solution;
drying the filled tips of the needle cavities to create releasable tips, and optionally annealing the needle tips;
filling the needle cavities of the mold with a dissolvable base solution;
drying the dissolvable base solution to create base layers for the releasable tips; and
applying a backing layer to the base layers to create a microneedle device.

75. The method of claim 74, further comprising removing the microneedle device from the mold.

76. The method of claim 75, wherein the microneedle device is removed by bending the mold away from the microneedle device.

77. The method of claim 75, further comprising packaging microneedle devices in a container with low moisture vapor transmission rate with a desiccant to maintain between about 0% and about 50% (e.g., between about 0% and 10%, between about 10% and about 20%, between about 20% and about 30%, between about 30% and about 40%, or between about 40% and 50%, e.g., about 25%) relative humidity inside the package.

78. The method of claim 74, wherein the silk fibroin, antigen solution is dispensed into each needle cavity in the mold via nanoliter printing.

79. The method of claim 78, wherein filling the tips of the needle cavities includes dispensing a solution, e.g., an antigen-silk formulation into each needle cavity.

80. The method of claim 74, wherein drying the filled tips of the needle cavities includes a primary drying step and a secondary drying step.

81. The method of claim 74, wherein filling the needle cavities of the mold with a dissolvable base solution includes a solution of 40% w/v Hydrolyzed Gelatin and 10% w/v Sucrose in DIW.

82. The method of claim 81, wherein drying the dissolvable base solution includes subjecting the mold to a centrifuge at 3900 rpm for 2 minutes and topping off the needle cavities with 50 μL of base solution.

83. The method of claim 74, further comprising an annealing step (e.g., before filling the base) after the filling the tips of the needle cavities.

84. The method of embodiment 74, further comprising a water annealing step (e.g., before filling the base) after the filling the tips of the needle cavities.

85. The method of claim 74, wherein the backing layer includes one of a paper backing layer and an adhesive plastic tape.

86. The use of a microneedle of any one of claims 1-59 in a method of providing immunity to a virus, e.g., an influenza virus.

87. The use of a microneedle of any one of claims 1-59 in a method of providing a sustained release of a vaccine, e.g., an influenza vaccine, in a subject.

88. The use of a microneedle of any one of claims 1-59 in a method of enhancing an immune response to a virus, e.g., an influenza virus, in a subject.

89. The microneedle of claim 1, wherein the dissolvable base comprising eight of gelatin, PEG, sucrose, CMC, PVP, PVA, hyaluronate, maltose, and methyl cellulose.

90. The microneedle of any one of the preceding claims, wherein the dissolvable base comprises PEG.

91. The microneedle of any one of the preceding claims, wherein the dissolvable base comprises between about 1% and about 70% polyethylene glycol (PEG) (e.g., about 1%, about 2%, about 3%, about 4%, about 5%, about 6%, about 7%, about 8%, about 9%, about 10%, about 20%, about 30%, about 40%, about 50%, about 60%, or about 70% PEG).

92. A method for providing broad-spectrum immunity to a virus, e.g., an influenza virus, in a subject, said method comprising administering a vaccine (e.g., a influenza vaccine) in an amount (e.g., a dosage) and/or over a time period sufficient to result in broad-spectrum immunity to a virus, e.g., results in an immune response (e.g., a cellular immune response and/or a humoral immune response) to a drifted strain of the virus, in the subject.

93. The method of claim 92, wherein the vaccine is administered in a composition for the controlled- or sustained-release of the vaccine (e.g., for the controlled- or sustained-release of one or more viral antigens as described herein).

94. The method of claim 92, wherein the vaccine is administered by a device for the controlled- or sustained-release of the vaccine (e.g., for the controlled- or sustained-release of one or more viral antigens as described herein).

95. The method of any of claims 92-94, wherein the vaccine is administered into a subject, e.g., in to a tissue or cavity of the subject chosen from skin, mucosa, organ tissue, muscle tissue or buccal cavity.

96. The method of any of claims 92-95, wherein the vaccine is administered in an amount (e.g., a dosage) and/or over a time period sufficient to result in one or more of:
(i) exposure in the subject to one or more antigens in the vaccine in an amount and/or period of time to result in broad spectrum immunity, e.g., to result in an immune response (e.g., a cellular immune response and/or a humoral immune response) to a drifted strain of the virus, in the subject; or
(ii) a level of one or more antigens in the subject that is substantially steady, e.g., about 20%, 15%, 10%, 5%, or 1% to an amount, e.g., minimum amount, needed to result in an immune response (e.g., a cellular immune response and/or a humoral immune response) to the one or more antigens.

97. The method of any one of claims 93-96, wherein the composition or device for the controlled- or sustained-release of the vaccine is chosen from: a microneedle (e.g., a microneedle device, e.g., a microneedle patch), an implantable device (e.g., a pump, e.g., a subcutaneous pump), an injectable formulation, a depot, a gel (e.g., a hydrogel), an implant, or a particle (e.g., a microparticle and/or a nanoparticle).

98. The method of claim 97, wherein the device for the controlled- or sustained-release of the vaccine comprises a microneedle or microneedle device, e.g., described herein.

99. The method of claim 97, wherein the device for the controlled- or sustained-release of the vaccine comprises a pump (e.g., a subcutaneous pump).

100. The method of claim 97, wherein the composition for the controlled- or sustained-release of the vaccine comprises an injectable formulation (e.g., an injectable depot formulation).

101. The method of claim 97, wherein the composition for the controlled- or sustained-release of the vaccine comprises an implant.

102. The method of claim 97, wherein the composition for the controlled- or sustained-release of the vaccine comprises a gel (e.g., a hydrogel).

103. The method of any one of claims 97-102, wherein the composition or device for the controlled- or sustained-release of the vaccine comprises a particle (e.g., a microparticle and/or a nanoparticle).

104. The method of any one of claims 92-104, wherein the vaccine is administered, e.g., released by the composition or device for the controlled- or sustained-release of the vaccine, e.g., into the subject, in order to maintain a vaccine dosage (e.g., an antigen concentration) for a period of time sufficient to result in broad spectrum immunity, e.g., to result in an immune response (e.g., a cellular immune response and/or a humoral immune response) to a drifted strain of the virus, in the subject (e.g., wherein the period of time is about 1 to 21 days, e.g., about 5 to 10 days or about 5 to 7 days, e.g., about 1, 2, 3, 4, 5, 6, 7, 8, 9, 10, 11, 12, 13, 14, 15, 16, 17, 18, 19, 20, or 21 days).

105. The method of claim 104, wherein the composition or device for the controlled- or sustained-release of the vaccine maintains antigen release and/or level in the subject over a sustained period of time.

106. The method of claim 104, wherein the composition or device for the controlled- or sustained-release of the vaccine maintains a continuous or non-continuous antigen release into the subject over a sustained period of time.

107. The method of any one of claims 92-106, wherein the vaccine is administered, e.g., released by the composition or device for the controlled- or sustained-release, over a period of time comprising at least about one week, e.g., about 1-2 weeks, about 1-3 weeks, or about 1-4 weeks.

108. The method of any one of claims 92-107, wherein the vaccine is administered, e.g., released by the composition or device for the controlled- or sustained-release, over a period of time comprising at least about 4 days (e.g., about 4, 5, 6, 7, 8, 9, 10, 11, 12, 13, 14, 15, 16, 17, 18, 19, or 20 days, or more, e.g., between about 4 days and about 2 weeks, between about 4 days and about 1 week).

109. The method of any one of claims 92-109, wherein the vaccine is administered in a dosage comprising between about 0.1 μg and about 65 μg per strain, e.g., 0.2 μg and about 50 μg per strain (e.g., about each of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 μg per strain).

110. The method of any one of claims 92-109, wherein at least about 1% of the dosage of the vaccine (e.g., at least about 0.5% to about 10%, at least about 5% to about 15% at least about 10% to about 20% of the dosage), e.g., released by the composition or device for the controlled- or sustained-release of the vaccine, e.g., into the subject, is maintained over a period of time comprising at least about 4 days (e.g., about 4, 5, 6, 7, 8, 9, 10, 11, 12, 13, or 14 days, or more, e.g., between about 4 days and about 2 weeks, between about 4 days and about 1 week).

111. The method of any one of claims 92-110, wherein the vaccine is administered, e.g., released by the composition or device for the controlled- or sustained-release, in a plurality of fractional doses of a total dose (e.g., a standard dose) over a time period, e.g., such that an immune response and/or broad-spectrum immunity is achieved,
wherein the amount of the vaccine administered in each of the fractional doses is no more than 1/X, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, or 100 or more, of the total dose (e.g., a standard dose) of the vaccine.

112. The method of any one of claims 92-110, wherein the vaccine is administered, e.g., released by the composition or device for the controlled- or sustained-release of the vaccine, e.g., into the skin of the subject, in a plurality of doses equivalent to a percentage of a total dose (e.g., a percentage of a standard dose) over a time period, e.g., such that broad-spectrum immunity is achieved,
wherein the amount of the vaccine administered in each of the plurality of doses is about X %, wherein X is any number, e.g., wherein X is 0.1, 0.2, 0.3, 0.4, 0.5, 0.6, 0.7, 0.8, 0.9, 1, 2, 3, 4, 5, 6, 7, 8, 9, 10, 15, 20, 30, 40, 50, 60, 70, 80, 90, 100, 125, 150, 175, 200, 300, 400, or 500 or more, of the total dose (e.g., a standard dose) of the vaccine.

113. The method of claim 111 or 112, wherein the vaccine is administered such that broad-spectrum immunity is achieved, e.g., such that an immune response, e.g., a cellular immune and/or humoral immune response to a drifted strain is achieved.

114. The method of claim 111 or 112, wherein the vaccine is administered as two, three, four, five, six, seven, eight, nine, ten or more fractional doses.

115. The method of any one of claims 111-114, wherein the total dose (e.g., the standard dose) of the vaccine is administered to achieve broad-spectrum immunity.

116. The method of any one of claims 111-114, wherein less than the total dose (e.g., the standard dose) of the vaccine is administered to achieve broad-spectrum immunity.

117. The method of any one of claims 111-114, wherein more than the total dose (e.g., the standard dose) of the vaccine is administered to achieve broad-spectrum immunity.

118. The method of any one of claims 111-114, wherein the amount of the vaccine administered in each of the fractional doses is the same.

119. The method of any one of claims 111-114, wherein the amount of the vaccine administered in each of the fractional doses is different.

120. The method of any one of claims 111-119, wherein the plurality of fractional doses is administered by intramuscular injection or intradermal injection, e.g., to achieve controlled- or sustained-release of a vaccine.

121. The method of any one of claims 111-120, wherein each dose of the plurality of fractional doses is administered at least once or twice a day, at least once every two days, at least once every three days, at least once every four days, at least once every five days, at least once every 6 days, at least one a week, or at least once a month for the duration of the time period.

122. The method of any one of claims 92-121, wherein:
(i) the vaccine comprises a first influenza strain and administration of a dose of the first influenza strain to the subject results in broad-spectrum immunity to a second influenza strain (e.g., a drifted influenza strain) not present in the implantable sustained-release tip or the vaccine;
(ii) the vaccine comprises a first influenza A strain and administration of a dose of the first influenza A strain to the subject results in broad-spectrum immunity to a drifted influenza strain (e.g., a drifted influenza A, B, C, and/or D strain) not present in the implantable sustained-release tip or the vaccine;
(iii) the vaccine comprises a first influenza B strain and administration of a dose of the first influenza B strain to the subject results in broad-spectrum immunity to a drifted influenza strain (e.g., a drifted influenza A, B, C, and/or D strain) not present in the implantable sustained-release tip or the vaccine;
(iv) the vaccine comprises a first influenza C strain and administration of a dose of the first influenza C strain to the subject results in broad-spectrum immunity to a drifted influenza strain (e.g., a drifted influenza A, B, C, and/or D strain) not present in the implantable sustained-release tip or the vaccine; and/or
(v) the vaccine comprises a first influenza D strain and administration of a dose of the first influenza D strain to the subject results in broad-spectrum immunity to a drifted influenza strain (e.g., a drifted influenza A, B, C, and/or D strain) not present in the implantable sustained-release tip or the vaccine.

123. The method of claim 122, wherein the first influenza A vaccine comprises:
(i) an H1N1 (e.g., A/Michigan and/or A/California) vaccine; and/or
(ii) an H3N2 (e.g., A/Hong Kong and/or A/Switzerland) vaccine.

124. The method of claim 122 or 123, wherein the drifted influenza A strain comprises:
(i) an H1N1 strain (e.g., A/Michigan and/or A/California); and/or
(ii) an H3N2 strain (e.g., A/Hong Kong and/or A/Switzerland).

125. The method of any one of claims 122-124, wherein:
(i) the first influenza A vaccine comprises an H1N1 vaccine to A/Michigan and the drifted influenza A strain comprises A/California; and/or
(ii) the first influenza A vaccine comprises an H3N2 vaccine to A/Hong Kong and the drifted influenza A strain is A/Switzerland.

126. The method of claim 122, wherein the first influenza B vaccine comprises:
(i) a B/Yamagata lineage strain (e.g., B/Phuket); and/or
(ii) a B/Victoria lineage strain (e.g., B/Brisbane).

127. The method of claim 122 or 126, wherein:
(i) the drifted influenza B strain is a B/Yamagata lineage strain (e.g., B/Phuket); and/or
(ii) the drifted influenza B strain is a B/Victoria lineage strain (e.g., B/Brisbane).

128. The method of any one of claim 122, 126, or 127, wherein the first influenza B vaccine is to the B/Victoria lineage strain B/Brisbane and the drifted influenza B strain is the B/Yamagata lineage strain B/Phuket.

129. The method of any one of claims 92-128, wherein the immune response and/or broad-spectrum immunity comprises a cellular and/or humoral immune response comprising:
(i) an elevated hemagglutination inhibition (HAI) antibody titer detectable in the blood of the subject, e.g., detectable at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization, optionally wherein the elevated HAI antibody titer is to a drifted influenza A, B, C, and/or D strain;
(ii) an elevated anti-influenza IgG titer detectable in the blood of the subject, e.g., detectable at least 1, 2, 3, 4, 5, 6, 7, 8, 9, 10, 11, and/or 12-months or more post immunization, optionally wherein the elevated anti-influenza IgG titer is to a drifted influenza A, B, C, and/or D strain; and/or
(iii) a level of antibody secreting plasma cells (ASC) against the virus, e.g., the influenza virus, e.g., the drifted influenza A, B, C, and/or D strain, detectable in the bone marrow of the subject, e.g., detectable at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization.

130. The method of claim 129, wherein an elevated hemagglutination inhibition (HAI) antibody titer is detectable in the blood of the subject for the duration of a complete flu season post immunization, optionally wherein the elevated HAI antibody titer is to a drifted influenza A, B, C, and/or D strain.

131. The method of claim 129, wherein the percent seroconversion, e.g., based on the elevated HAI antibody titer detectable in the blood of the subject, e.g., at 6-month post immunization is greater than about 20% (e.g., 20%, 25%, 30%, 35%, 40%, 45%, 50%, 55%, 60%, 65%, 70%, 75%, 80%, 85%, 90%, or 95% or more, e.g., 100%).

132. The method of any one of claims 92-131, wherein broad-spectrum immunity comprises a cellular immune response comprising an increase in the level of IFNγ secreting cell in the blood of the subject, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, and/or 52-weeks or more post immunization.

133. The method of any one of claims 109-132, wherein the elevated HAI antibody titer, the elevated anti-influenza IgG titer, the level of antibody secreting plasma cells (ASC) against the virus, and/or the level of IFNγ secreting cells detectable in the subject is greater (e.g., 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold or more greater) as compared to the administration of a single-dose or a bolus administration of the vaccine.

134. A method for providing an immune response (e.g., a cellular immune response and/or a humoral immune response) and/or a broad spectrum immunity to a virus, e.g., an influenza virus, in a subject, said method comprising administering a vaccine (e.g., a influenza vaccine) in an amount (e.g., a dosage) and/or over a time period sufficient to elicit an immune response (e.g., a cellular immune response and/or a humoral immune response) to the virus, e.g., the influenza virus, in the subject,
wherein the vaccine is administered in a composition for the controlled- or sustained-release of the vaccine (e.g., for the controlled- or sustained-release of one or more viral antigens as described herein) over a period of time comprising about 1 to about 2 weeks (e.g., about 10 days).

135. A method or vaccine regimen of any one of the preceding claims, wherein the subject (e.g., the human subject) is a pediatric subject.

136. A method or vaccine regimen of any one of the preceding claims, wherein the subject (e.g., the human subject) is an adult subject.

137. A method or vaccine regimen of any one of the preceding claims, wherein the subject (e.g., the human subject) is an elderly subject.
